SEQUENCE LISTING (1 sequence)

SEQ ID NO: 1 - protein, 157 aa, Mus musculus
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

(19) United States
(12) Patent Application Publication
(10) Pub. No.: US 2006/0177871 A1
(43) Pub. Date: Aug. 10, 2006

(54) Murine IL-18 crystal structure

(76) Inventors: Cheryl A. Janson, Hinsdale, IL (US); Xiayang Qiu, Mystic, CT (US); Ward Smith, Hinsdale, IL (US)

Correspondence Address: GLAXOSMITHKLINE;Corporate Intellectual Property - UW2200, P.O. Box 1539, King of Prussia, PA, 19406-0939, US

(21) Appl. No.: 10/640,524

(22) Filed: Aug. 13, 2003

Related U.S. Application Data
(60) Provisional application No. 60/403,077, filed on Aug. 13, 2002.

(51) Int. Cl. G01N 33/53; G06F 19/00; C07K 14/54

(52) U.S. Cl. 435/7.1; 530/351; 702/19

(57) ABSTRACT

A novel murine IL-18 native crystalline structure is identified.

### CROSS-REFERENCE TO RELATED APPLICATION

This application claims priority to the earlier provisional U.S. application, Ser. No. 60/403,077, which was filed on Aug. 13, 2002, the contents of which are herein incorporated by reference in their entirety.

### FIELD OF THE INVENTION

The invention relates to the identification of a novel crystalline structure of the murine IL-18 (mIL-18) cytokine, its mode of binding to its receptor, and methods enabling further design and selection of molecules with mIL-18-like activity.

### BACKGROUND OF THE INVENTION

IL-18 is a type of cytokine or substance that mediates signal transduction in the immune system. As seen in Japanese Patent Kokai Nos.27,189/96 and 193,098/96 and Okamura et al., Nature, Vol. 378, No. 6,552, pp. 88-91 (1995), IL-18 was provisionally designated as “interferon-gamma inducing factor” immediately after its discovery. This designation was later changed into “IL-18” in accordance with the proposal in Ushio, et al., Journal of Immunology, Vol. 156, pp. 4,274-4,279 (1996). IL-18 in its mature form consists of 157 amino acids. It induces immunocompetent cells in the production of interferon-gamma (hereinafter abbreviated as “IFN-gamma.”), which is a useful biologically-active protein capable of inducing and enhancing the generation and cytotoxicity of killer cells. Extensive research is currently underway to develop and explore the various utility of IL-18 in pharmaceuticals. These greatly expected applications include using IL-18 as antiviral, antimicrobial, antitumor and anti-immunopathic agents.

In nature, cytokines, including IL-18, are produced and secreted as substances responsible for signal transduction in the immune system. Therefore, when cytokines are administered to the body of mammals, they disturb the naturally existing equilibrium in the mammal's immune system. The surfaces of mammalian cells bear sites or “receptors” that are responsible for recognition of cytokines and secreted cytokines transduce no signal in cells until they are bound to the receptors. In a normal immune system, a definite equilibrium exists between respective cytokines and their receptors. There are currently unmet needs in finding and learning the biological and structural properties of IL-18 and its receptors and using such knowledge in designing drugs for treatment and ameliorating diseases and disorders such as viral and microbial infections, cancer, inflammation, etc.

### SUMMARY OF THE INVENTION

In one aspect, the present invention relates to a murine IL-18 protein molecule having the coordinates of Table I in an essentially pure native form or a homolog thereof.

In another aspect, the present invention provides a novel crystalline form of the murine IL-18 molecule.

In yet another aspect, the present invention provides direct information on the specific role played by the residues responsible for the binding of murine IL-18 to its receptor.

In a further aspect, the present invention includes machine-readable media encoded with data representing the coordinates of the three-dimensional structure of the murine IL-18 crystal.

### DETAILED DESCRIPTION OF THE INVENTION

The present invention provides a novel murine IL-18 crystalline structure of the native protein. Based on this structure and molecular models built using related proteins, it provides ways of determining the most likely places to modify the molecule of mIL-18 without compromising its biological activity and methods to use this crystalline form in identifying, improving or antagonizing the biological activity of mIL-18.

The Novel Murine IL-18 Crystalline Three-Dimensional Structure

The crystal structure of the murine IL-18 in its native form has been determined by molecular replacement and refined to 1.55 Å resolution. The novel murine IL-18, like IL1β, is folded into a central, closed β-barrel with an overall β-trefoil fold. The following residues form the three parts of the clover-leaf: (i) 10-46, (ii)103-152, and (iii) 47-102 and 1-9. The structure of human IL-18 is expected to be similar to that of the murine IL-18 since the sequences of these IL-18s are highly homologous (65% identity). The information derived from the structure of murine IL-18 sheds light on how complexes with pharmacological agents may be formed that would alter the properties of human IL-18, such as half-life and immunogenicity, while maintaining its biological activity. In the absence of structural information of the IL-18-receptor complex, the mIL-18 structure and that of the IL1β-IL1β receptor complex (PDB:11TB, G. P. Vigers et al. (1997) Nature 386, 190) are used as models for interactions between IL-18 and its receptor and provide rational guidance as to where to place potential agents in the IL18 molecule (see FIG. 3). IL1β and IL-18 have similar overall folds and structures, but large differences in the two structures are apparent near and at the positions of loops. In the IL1β-receptor complex, the residues in the IL1β loops establish important interactions with the receptor, mainly through two surfaces. To identify the residues of IL-18 that may interact with IL-18 receptor, residues of IL-18 were mapped onto the IL1β structure by superimposing 153 Ca atoms of IL1β onto the Cα atoms of IL-18 to achieve an overall root mean square (r.m.s.) deviation of 7 Å. Based on this superposition, IL-18 is predicted to interact with its receptor via several surfaces. On one of the proposed interacting surfaces, the receptor-IL-18 interface is lined by IL-18 residues: 15-18, 22-27, 31-36, 128-131, and 145-148. On another proposed surface located on the other side of the molecule, the interface is lined by IL-18 residues: 3-8, 50-58 and 89-92. A third proposed surface is located between the previous two and includes residues 103-109 of the IL-18 molecule. This leaves residues 38-41, 67-70, 76-88, 117-120 and 140-142 in mIL-18 free from interactions with the receptor, and these residues are mostly solvent exposed and are potentially positions to attach a derivatization agent, such as a polyethyleneglycol molecule, whereas said agent does not compromise IL-18's binding to its receptor.

In a preferred embodiment, this invention relates to the crystal of murine IL-18, wherein said crystal effectively diffracts x-rays for the determination of the atomic coordinates of said murine IL-18 to a resolution of between about 1.3 Å and 5.0 Å. In a most particularly preferred embodiment, the resolution is between about 1.3 Å and 3.0 Å. In a most particularly preferred embodiment, the resolution of between about 1.3 Å and 2.5 Å.

Table I provides the atomic coordinates of the native murine IL-18. These coordinates were obtained using a model encompassing residues 1 to 152 in the crystallographic asymmetric unit. The amino acid sequence of the native murine IL-18 is provided in SEQ ID NO: 1. Residues 153-157 are not visible in the x-ray determined structure of the wild type molecule.

The atomic coordinates shown in Table I are expected to change upon refinement of the crystal structure, but the deviation that would incur as a result with regard to the Cα atoms is not expected to substantially exceed an r.m.s. of 1.0-1.5 Å. Similarly, bond angles and bond lengths will vary insignificantly as routinely observed with other proteins. Engh, et al. (1991) Acta Crystallogr. A47, 392400. The inter-atomic interactions will remain unchanged, within experimental error. The relative conformation and orientation or the positioning of residues in the receptor-binding site will likewise be unaffected.

### DESCRIPTION OF THE FIGURES

FIG. 1 provides a ribbon diagram of native murine IL-18 of this invention with the view taken perpendicular to the axis of the β-barrel and the helical segments represented as ribbons and the β-sheets represented as arrows. The polypeptide chain is rainbow colored with the color blue at the N-terminus and the color red at the C-terminus.

FIG. 2 provides diagrams of murine IL-18, human IL-1β, and human IL-18 structures. Side-by-side comparison of these structures shows the conformational similarities between these structurally related cytokines.

FIG. 3 provides a diagram where the mIL-18 model is superimposed on the IL1β-IL1 receptor complex. Murine IL-18 is drawn in a green line and the IL1β-IL1 complex is drawn in a yellow line as the cytokine and a red line as the receptor. This diagram suggests the manner in which mIL-18 binds its receptor and the amino acid interactions between the cytokine and the receptor.

### MUTANTS AND DERIVATIVES

The invention further provides homologues, co-complexes, mutants and derivatives of the murine IL-18 crystal structure of the invention.

The term “cytokine”, as used herein, means a protein modulating the growth and functional activities of immune cells.

The term “homolog”, as used herein, means a protein having at least 30% amino acid sequence identity with a functional domain of murine IL-18. Preferably the percentage identity will be 40, or 50%, more preferably 60 or 70% and most preferably 80 or 90%. A 95% identity is most particularly preferred.

The term “co-complex”, as used herein, means the murine IL-18 or a mutant or homologue of the murine IL-18 in covalent or non-covalent association with a chemical entity or compound.

The term “mutant”, as used herein, means the murine IL-18 polypeptide, i.e., a polypeptide displaying the biological activity of wild-type murine IL-18 activity, characterized by the replacement of at least one amino acid from the wild-type IL-18 sequence. Such a mutant may be prepared, for example, by expression of the murine IL-18 cDNA previously altered in its coding sequence by oligonucleotide-directed mutagenesis.

The term “r.m.s.”, as used herein, means root mean square. It represents the standard deviation of the data collection.

The term “pro-IL18”, as used herein, means the inactive, precursor form of mature IL18. Mature IL18 does not contain the “pro-fragment” and is biologically active.

The term “molecular replacement”, as used herein, means a method of solving crystal structure using the atomic coordinates of a structurally related molecule.

Murine IL-18 mutants may also be generated by site-specific incorporation of unnatural amino acids into the murine IL-18 protein using the general biosynthetic method of Noren, et al., Science, 244:182-188 (1989). In this method, the nucleotides encoding the amino acid of interest in wild-type murine IL-18 is replaced by a “blank” nonsense codon, TAG, using oligonucleotide-directed mutagenesis. A suppressor directed against this codon is then chemically aminoacylated in vitro with the desired unnatural amino acid. The aminoacylated residue is then added to an in vitro translation system to yield a mutant murine IL-18 enzyme with the site-specific incorporated unnatural amino acid.

Selenocysteine or selenomethionine may be incorporated into wild-type or mutant cytokine by expression of murine IL-18-encoding cDNAs in auxotrophic E. coli strains. Hendrickson, et al., EMBO J., 9(5):1665-1672 (1990). In this embodiment, the wild-type or mutated cytokine cDNA may be expressed in a host organism on a growth medium depleted of either natural cysteine or methionine or both, but enriched with selenocysteine or selenomethionine or both.

The term “heavy atom derivative” refers to derivatives of murine IL-18 produced by chemically modifying a crystal of murine IL-18. In practice, a crystal is immersed in a solution containing the desired metal salt, or organometallic compound, e.g., lead chloride, gold thiomalate, thimerosal or uranyl acetate, which upon diffusion into the protein crystal can bind to the protein. The location of the bound heavy metal atom site(s) can be determined by X-ray diffraction analysis of the soaked crystal. This information, in turn, is used to generate the phase angle information needed to construct a three-dimensional electron density map from which a model of the atomic structure of the enzyme is derived Blundell, et al., PROTEIN CRYSTALLOGRAPHY, Academic Press (1976).

Methods of Identifying Agonist or Antagonists of the Novel Murine IL-18 Crystalline Structure

Another aspect of this invention involves a method for identifying agonists or antagonists of a murine IL-18 through the crystal structure described herein. The novel murine IL-18 crystalline structure of the invention permits the identification of agonists or antagonists of its cytokine activity. Such agonist/antagonists may be competitive, binding to all or a portion of the receptor for the murine IL-18; or non-competitive and bind to and inhibit IL-18 activity whether or not it is bound to the receptor.

One embodiment probes the murine IL-18 crystal of the invention with a variety of different chemical molecules to determine optimal sites either for interactions between such candidate angonist/antagonist molecules and mIL-18, or alternatively, for cellular activities. For example, high resolution X-ray diffraction data collected from crystals saturated with solvent allows the determination of binding positions for solvent molecule. Small molecules that would bind tightly to those sites can then be designed, synthesized and tested for their murine IL-18 agonist/antagonist activities.

Another embodiment screens small molecule databases computationally for chemical entities or compounds that can bind in whole, or in part, to murine IL-18 or murine IL-18 receptor, or both. This screening method and its utility are well known in the art. For example, such computer modeling techniques were described in a PCT application WO 97/16177, published on May 9, 1997.

Once identified by modeling, the agonist/antagonist may then be tested for biological activity. For example, the molecules identified may be introduced via standard screening formats into enzymatic activity assays to determine the inhibitory activity of the compounds, or alternatively, binding assays to determine binding. One particularly preferred assay format is the enzyme-linked immunosorbent assay (ELISA). This and other assay formats are well known in the art and thus are not limitations to the present invention.

The following examples illustrate various aspects of this invention. This invention is not to be limited in scope by the specific embodiments described below. Indeed, various modifications of the invention in addition to those described herein will become apparent to those skilled in the art from the foregoing description. Such modifications are intended to fall within the scope of the appended claims. The disclosures of patents, patent applications and publications cited herein are incorporated by reference in their entireties.

### EXAMPLES/BIOLOGICAL METHODS

### Example 1

### The Purification of the Murine IL-18 Mutant C7S-Q37C from Escherichia coli

Murine proIL-18 was overexpressed in E. coli as a soluble protein with an N-terminal hexa His tag. ProIL-18 was purified with a Ni-NTA affinity column, followed by a DEAE-650M column. This purified proIL-18 was then cleaved to become mature IL-18 using caspase 1. The cleavage product was applied to a second Ni-NTA column and only the mature form of murine IL-18 flows through while the uncleaved proIL-18 is bound to the column. The mature IL-18 was further purified using a Superdex 75 column, from which it was eluted in the form of a monomer. This was followed by a high-resolution monoQ column so that the mature murine IL-18 was sufficiently pure for crystallography studies.

E. coli cells were suspended at 3 ml/g in 50 mM Tris (pH 8), 0.5M NaCl, 10 mM β-mercaptoethanol, 5% glycerol, 1 μg/ml pepstatin A, 1 μg/ml leupeptin, 0.4mM AEBSF. Cells were homogenized and then lysed at 12,000 psi with a microfluidizer (M110-Y, Microfluidics, Inc.). The cell lysate was then centrifuged at 30,000×g for 20 minutes. HexaHis tagged proIL-18 from the supernatant was applied to a Ni-NTA column (Qiagen, Inc.) in the presence of 5 mM imidazole. The Ni-NTA was extensively washed (more than 5 column volumes) with 50 mM Tris (pH 8), 0.5M NaCl, 10 mM β-mercaptoethanol and 5 mM imidazole. ProIL-18 was eluted from the column with 30 mM imidazole. The HexaHis proIL-18 in the Ni-NTA eluate was dialyzed against a buffer of 50 mM Tris (pH 8) and 10 mM β-mercaptoethanol. The dialyzed solution was then centrifuged at 30,000×g for 10 minutes and applied to a Toyopearl DEAE 650M column. HexaHis proIL-18 was eluted with a linear salt gradient of 0 to 0.5M NaCl in 25 mM HEPES (pH 8) and 10 mM β-Mercaptoethanol.

ProIL-18 was cleaved using caspase 1 to generate mature L-18 for 5.5 hours at room temperature at a weight ratio of ProIL 8:caspase 1=1:500. The cleaved mixture containing the pro-domain and uncleaved HexaHis proIL-18 were applied to a second Ni-NTA column, whereby the pro-domain and the uncleaved proIL-18 was captured. The flow through fractions containing mature IL-18 was concentrated and treated with 40 mM DTT at pH 8 for 2 hours. Thereafter, the mature L-18 solution was adjusted to pH 6 using 1M phosphoric acid. This solution was centrifuged at 30,000×g before it was applied to a Superdex 75 column equilibrated with 10 mM NaPi (pH 6) and 0.1M NaCl. The monomer peak from the Superdex 75 column eluate was pooled and further fractioned by a MonoQ HR 10/10 column. Mature L-18 was eluted with a linear salt gradient of 0 to 0.25M NaCl in 25 mM Tris (pH 7.5) and 5 mM β-Mercaptoethanol. Mature IL-18 was then pooled and dialyzed against a buffer of 25mM Tris (pH 7.5), 50 mM NaCl, 0.1 mM EDTA and 5 mM β-Mercaptoethanol and concentrated to 11 mg/ml.

The invention described herein provides a method for defining ligand interactions with L-18 and its receptor:

1.A. Effects of ligand binding upon enzyme intrinsic fluorescence generated by tryptophan residues. Binding of either a natural ligand or a derivatized molecule may result in conformational changes that alter protein intrinsic fluorescence. Using stopped-flow fluorescence technology, one can use this change in intrinsic fluorescence to define the microscopic rate constants that are associated with ligand binding. Alternatively, one can use steady-state fluorescence titration methods to generate the overall dissociation constant for binding. Standard methods are applied in assessing the acquired parameters.

### Example 2

### Crystallization, Structure Determination and Refinement of the Crystal Structure of Murine IL-18

2.A. Crystallization

The murine IL-18 native crystals grew from sitting or hanging drops equilibrated through the vapor phase at room temperature with a reservoir of 500 μL reservoir solution containing 23-30% polyethylene glycol (PEG) 1500 in 1-6 weeks. The drops contained 2 μL of protein at 10-30 mg/ml in 50 mM NaCl, 25 mM Tris (pH 7.4), 0.1 mM EDTA and 2 μL reservoir solution. Form I crystals belong to the space group P2₁2₁2 with unit cell dimensions of a=71.3 Å, b=96.5 Å, c=50.8 Å with two molecules per asymmetric unit. Form II crystals belong to the space group P2₁2₁2₁ with unit cell dimensions a=36.0 Å, b=61.2 Å, c=65.3 Å and one molecule in the asymmetric unit.

The mutant murine IL-18 (C7S, Q37C) crystals grew from sitting or hanging drops equilibrated through the vapor phase at room temperature against a reservoir of 500 μL reservoir solution containing 18-30% polyethylene glycol (PEG) 1500 with 0 or 2-5 mM Tris (pH 8) in seven days. The drops contained 2 μL of protein at 10 mg/ml in 50 mM NaCl, 25 mM Tris (pH 7.5), 0.1 mM EDTA, 5 mM β-Mercaptoethanol and 2 μL reservoir solution. The crystals belong to the space group P2₁2₁2₁ with unit cell dimensions of a=36.0 Å, b=61.3 Å, c=65.3 Å and one copy of the murine IL-18 C7S, Q37C mutant protein in the asymmetric unit.

2.B. Heavy Atom Derivatization

Form I native crystal was used to form heavy atom derivatives. These heavy atoms provided weak derivatization and the data collected from them were used to resolve the native murine IL-18 structure. A saturated solution of MeHgCl was used to soak a crystal for 2 hours in the first derivative. A saturated solution of PCMB was used to soak a second crystal for 5 hrs in the second derivative. A saturated solution of Baker's dimercurial was used to soak a third crystal for 4 hrs in the third derivative.

2.C. X-ray Diffraction Data Collection

We collected data from the Form I native murine IL-18 crystal and the heavy atom derivative crystals (MeHgCl, pCMB and Baker's dimercurial) at room temperature on an R-axis image plate using x-rays generated by a Rigaku RU200 rotating anode. The data were then processed with an HKL program as described by Otwinowski, et al. in the Methods in Enzymology, 276:307-326, Carter, Jr. and Sweet, eds., Academic Press, New York.

The C7S-Q37C mutant crystals were soaked in a cryo-solution containing 80% reservoir solution and 20% ethylene glycol. A Form II mutant crystal was then suspended in a nylon loop and frozen under a nitrogen gas stream. Likewise, the Form II native crystal was suspended in a nylon loop, dipped in paratone-N oil and frozen under a nitrogen gas stream.

Data from the Form II native crystal and the mutant crystal were collected with a MAR CCD detector on beamline 17ID, the Advanced Photon Source at Argonne National Laboratory. The data were processed with HKL2000 as described by Otwinowski, in the Proceedings of the CCP4 Study Weekend: “Data Collection and Processing”, Daresbury, England (1993).

2.D. Structure Determination and Refinement

The crystal structure of murine IL-18 was determined by the heavy atom method using a computer program named SOLVE as described by Terwilliger, et al. in Acta Crystallogr. D 55:849-861 (1999). The structure of each derivative was determined individually and then in combination with the heavy atom information using phase combination. The resulting map was solvent flattened using a program named DM (CCP4, 1993). The backbone of the molecule was then incorporated into this electron density map using a program named XTALVIEW. McRee, Practical Protein Crystallography, Academic Press, San Diego (1993). This backbone provided a structural model for the murine protein in the deter. Using this model and molecular replacement program AmoRe (Navaza, Acta Cryst. A 50:157-163 (1994)), we determined that the Form II mutant IL-18 contains one monomer in the asymmetric unit. This structure was refined using ARP/wARP as described by Perrakis, et al. in Acta Cryst. D53, 448455 (1997) and further refined with a program named CNX (by Accelrys) to final R-factors of R=18.88% and R_{free}=21.37% for 29188 reflections at 1.35 Å resolution. The mutant murine IL-18 (C7S-Q37C) contains residues 1-157 and 243 water molecules.

Using the mutant structure as a search model, the structure of the Form II native crystal was determined by molecular replacement AmoRe. Like the Form II mutant crystals, the native Form II crystal coordinates were built using ARP/wARP and further refined using CNX to final R-factors of R=20.21% and R_{free}=23.96% for 20885 reflections at a resolution of 1.55 Å. The native murine IL-18 contain residues 1-152 and 230 water molecules. Table I lists the final coordinates of the native murine IL-18.

TABLE I
Atomic coordinates of the native murine IL-18 structure

| | | | | | | | | | |
|---|---|---|---|---|---|---|---|---|---|
| 1 | CB | ASN | A | 1 | −9.865 | 20.729 | −8.348 | 1.00 | 22.11 |
| 2 | CG | ASN | A | 1 | −10.876 | 19.626 | −8.175 | 1.00 | 25.16 |
| 3 | OD1 | ASN | A | 1 | −12.086 | 19.881 | −8.154 | 1.00 | 26.62 |
| 4 | ND2 | ASN | A | 1 | −10.398 | 18.389 | −8.061 | 1.00 | 24.23 |
| 5 | C | ASN | A | 1 | −9.275 | 20.938 | −5.896 | 1.00 | 18.24 |
| 6 | O | ASN | A | 1 | −9.498 | 20.013 | −5.125 | 1.00 | 17.59 |
| 7 | N | ASN | A | 1 | −8.019 | 19.368 | −7.404 | 1.00 | 20.53 |
| 8 | CA | ASN | A | 1 | −8.739 | 20.674 | −7.308 | 1.00 | 19.06 |
| 9 | N | PHE | A | 2 | −9.460 | 22.217 | −5.564 | 1.00 | 17.55 |
| 10 | CA | PHE | A | 2 | −9.910 | 22.648 | −4.242 | 1.00 | 16.07 |
| 11 | CB | PHE | A | 2 | −8.914 | 23.651 | −3.661 | 1.00 | 17.36 |
| 12 | CG | PHE | A | 2 | −7.540 | 23.107 | −3.461 | 1.00 | 13.98 |
| 13 | CD1 | PHE | A | 2 | −7.213 | 22.388 | −2.295 | 1.00 | 14.03 |
| 14 | CD2 | PHE | A | 2 | −6.555 | 23.345 | −4.405 | 1.00 | 14.18 |
| 15 | CE1 | PHE | A | 2 | −5.903 | 21.921 | −2.083 | 1.00 | 11.11 |
| 16 | CE2 | PHE | A | 2 | −5.239 | 22.883 | −4.206 | 1.00 | 12.74 |
| 17 | CZ | PHE | A | 2 | −4.915 | 22.172 | −3.040 | 1.00 | 12.98 |
| 18 | C | PHE | A | 2 | −11.244 | 23.371 | −4.332 | 1.00 | 17.48 |
| 19 | O | PHE | A | 2 | −11.463 | 24.128 | −5.275 | 1.00 | 20.98 |
| 20 | N | GLY | A | 3 | −12.104 | 23.160 | −3.346 | 1.00 | 16.12 |
| 21 | CA | GLY | A | 3 | −13.402 | 23.824 | −3.332 | 1.00 | 17.13 |
| 22 | C | GLY | A | 3 | −13.526 | 24.559 | −2.015 | 1.00 | 16.98 |
| 23 | O | GLY | A | 3 | −13.316 | 23.967 | −0.955 | 1.00 | 17.79 |
| 24 | N | ARG | A | 4 | −13.889 | 25.836 | −2.065 | 1.00 | 18.57 |
| 25 | CA | ARG | A | 4 | −13.968 | 26.618 | −0.840 | 1.00 | 17.15 |
| 26 | CB | ARG | A | 4 | −14.078 | 28.099 | −1.187 | 1.00 | 16.30 |
| 27 | CG | ARG | A | 4 | −13.691 | 28.996 | −0.022 | 1.00 | 17.00 |
| 28 | CD | ARG | A | 4 | −13.838 | 30.429 | −0.369 | 1.00 | 19.79 |
| 29 | NE | ARG | A | 4 | −15.240 | 30.838 | −0.372 | 1.00 | 21.81 |
| 30 | CZ | ARG | A | 4 | −15.633 | 32.073 | −0.650 | 1.00 | 25.07 |
| 31 | NH1 | ARG | A | 4 | −14.740 | 32.999 | −0.954 | 1.00 | 23.73 |
| 32 | NH2 | ARG | A | 4 | −16.914 | 32.394 | −0.572 | 1.00 | 27.29 |
| 33 | C | ARG | A | 4 | −15.121 | 26.190 | 0.060 | 1.00 | 19.88 |
| 34 | O | ARG | A | 4 | −16.243 | 25.991 | −0.408 | 1.00 | 21.53 |
| 35 | N | LEU | A | 5 | −14.828 | 26.027 | 1.346 | 1.00 | 19.15 |
| 36 | CA | LEU | A | 5 | −15.816 | 25.620 | 2.340 | 1.00 | 20.48 |
| 37 | CB | LEU | A | 5 | −15.292 | 24.429 | 3.165 | 1.00 | 22.10 |
| 38 | CG | LEU | A | 5 | −14.905 | 23.191 | 2.324 | 1.00 | 24.19 |
| 39 | CD1 | LEU | A | 5 | −14.284 | 22.118 | 3.173 | 1.00 | 25.88 |
| 40 | CD2 | LEU | A | 5 | −16.135 | 22.654 | 1.632 | 1.00 | 25.55 |
| 41 | C | LEU | A | 5 | −16.063 | 26.851 | 3.219 | 1.00 | 19.93 |
| 42 | O | LEU | A | 5 | −16.131 | 27.964 | 2.698 | 1.00 | 24.40 |
| 43 | N | HIS | A | 6 | −16.184 | 26.684 | 4.527 | 1.00 | 21.35 |
| 44 | CA | HIS | A | 6 | −16.447 | 27.860 | 5.350 | 1.00 | 21.14 |
| 45 | CB | HIS | A | 6 | −17.412 | 27.526 | 6.503 | 1.00 | 23.05 |
| 46 | CG | HIS | A | 6 | −16.775 | 26.810 | 7.657 | 1.00 | 27.34 |
| 47 | CD2 | HIS | A | 6 | −16.361 | 27.264 | 8.866 | 1.00 | 27.83 |
| 48 | ND1 | HIS | A | 6 | −16.509 | 25.459 | 7.644 | 1.00 | 28.07 |
| 49 | CE1 | HIS | A | 6 | −15.962 | 25.108 | 8.796 | 1.00 | 30.59 |
| 50 | NE2 | HIS | A | 6 | −15.860 | 26.186 | 9.555 | 1.00 | 29.47 |
| 51 | C | HIS | A | 6 | −15.194 | 28.505 | 5.908 | 1.00 | 21.75 |
| 52 | O | HIS | A | 6 | −14.094 | 27.957 | 5.803 | 1.00 | 20.28 |
| 53 | N | CYS | A | 7 | −15.361 | 29.693 | 6.480 | 1.00 | 20.13 |
| 54 | CA | CYS | A | 7 | −14.221 | 30.384 | 7.067 | 1.00 | 19.41 |
| 55 | CB | CYS | A | 7 | −13.979 | 31.755 | 6.406 | 1.00 | 20.20 |
| 56 | SG | CYS | A | 7 | −15.383 | 32.948 | 6.489 | 1.00 | 22.66 |
| 57 | C | CYS | A | 7 | −14.443 | 30.594 | 8.541 | 1.00 | 21.66 |
| 58 | O | CYS | A | 7 | −15.559 | 30.503 | 9.037 | 1.00 | 21.74 |
| 59 | N | THR | A | 8 | −13.335 | 30.827 | 9.232 | 1.00 | 18.67 |
| 60 | CA | THR | A | 8 | −13.316 | 31.143 | 10.652 | 1.00 | 18.54 |
| 61 | CB | THR | A | 8 | −12.817 | 29.988 | 11.513 | 1.00 | 18.35 |
| 62 | OG1 | THR | A | 8 | −11.560 | 29.526 | 11.015 | 1.00 | 22.80 |
| 63 | CG2 | THR | A | 8 | −13.831 | 28.849 | 11.509 | 1.00 | 21.68 |
| 64 | C | THR | A | 8 | −12.302 | 32.266 | 10.719 | 1.00 | 17.98 |
| 65 | O | THR | A | 8 | −11.605 | 32.542 | 9.741 | 1.00 | 17.69 |
| 66 | N | THR | A | 9 | −12.223 | 32.943 | 11.849 | 1.00 | 18.04 |
| 67 | CA | THR | A | 9 | −11.251 | 34.001 | 11.947 | 1.00 | 18.97 |
| 68 | CB | THR | A | 9 | −11.883 | 35.369 | 12.261 | 1.00 | 21.56 |
| 69 | OG1 | THR | A | 9 | −12.507 | 35.334 | 13.547 | 1.00 | 23.78 |
| 70 | CG2 | THR | A | 9 | −12.907 | 35.739 | 11.199 | 1.00 | 22.25 |
| 71 | C | THR | A | 9 | −10.303 | 33.622 | 13.055 | 1.00 | 19.38 |
| 72 | O | THR | A | 9 | −10.655 | 32.881 | 13.976 | 1.00 | 19.63 |
| 73 | N | ALA | A | 10 | −9.079 | 34.114 | 12.943 | 1.00 | 16.88 |
| 74 | CA | ALA | A | 10 | −8.079 | 33.831 | 13.963 | 1.00 | 15.66 |
| 75 | CB | ALA | A | 10 | −7.407 | 32.482 | 13.715 | 1.00 | 17.32 |
| 76 | C | ALA | A | 10 | −7.024 | 34.893 | 13.962 | 1.00 | 14.99 |
| 77 | O | ALA | A | 10 | −6.767 | 35.540 | 12.959 | 1.00 | 13.33 |
| 78 | N | VAL | A | 11 | −6.410 | 35.065 | 15.118 | 1.00 | 14.32 |
| 79 | CA | VAL | A | 11 | −5.290 | 35.971 | 15.264 | 1.00 | 14.19 |
| 80 | CB | VAL | A | 11 | −5.375 | 36.764 | 16.559 | 1.00 | 14.11 |
| 81 | CG1 | VAL | A | 11 | −4.050 | 37.503 | 16.813 | 1.00 | 14.59 |
| 82 | CG2 | VAL | A | 11 | −6.525 | 37.758 | 16.456 | 1.00 | 14.05 |
| 83 | C | VAL | A | 11 | −4.119 | 34.978 | 15.295 | 1.00 | 13.53 |
| 84 | O | VAL | A | 11 | −4.154 | 33.954 | 16.005 | 1.00 | 15.49 |
| 85 | N | ILE | A | 12 | −3.090 | 35.261 | 14.510 | 1.00 | 13.01 |
| 86 | CA | ILE | A | 12 | −1.955 | 34.388 | 14.383 | 1.00 | 12.82 |
| 87 | CB | ILE | A | 12 | −1.592 | 34.216 | 12.898 | 1.00 | 14.30 |
| 88 | CG2 | ILE | A | 12 | −0.335 | 33.410 | 12.746 | 1.00 | 14.56 |
| 89 | CG1 | ILE | A | 12 | −2.758 | 33.552 | 12.173 | 1.00 | 16.31 |
| 90 | CD1 | ILE | A | 12 | −2.482 | 33.278 | 10.719 | 1.00 | 16.85 |
| 91 | C | ILE | A | 12 | −0.764 | 34.947 | 15.124 | 1.00 | 11.30 |
| 92 | O | ILE | A | 12 | −0.408 | 36.118 | 14.952 | 1.00 | 12.05 |
| 93 | N | ARG | A | 13 | −0.175 | 34.111 | 15.970 | 1.00 | 12.36 |
| 94 | CA | ARG | A | 13 | 1.005 | 34.540 | 16.709 | 1.00 | 10.15 |
| 95 | CB | ARG | A | 13 | 0.736 | 34.519 | 18.204 | 1.00 | 11.19 |
| 96 | CG | ARG | A | 13 | −0.109 | 35.692 | 18.633 | 1.00 | 10.74 |
| 97 | CD | ARG | A | 13 | −0.550 | 35.548 | 20.075 | 1.00 | 12.36 |
| 98 | NE | ARG | A | 13 | −1.301 | 36.718 | 20.510 | 1.00 | 12.86 |
| 99 | CZ | ARG | A | 13 | −2.614 | 36.859 | 20.390 | 1.00 | 13.50 |
| 100 | NH1 | ARG | A | 13 | −3.363 | 35.896 | 19.853 | 1.00 | 15.13 |
| 101 | NH2 | ARG | A | 13 | −3.177 | 37.988 | 20.821 | 1.00 | 14.39 |
| 102 | C | ARG | A | 13 | 2.190 | 33.651 | 16.383 | 1.00 | 12.82 |
| 103 | O | ARG | A | 13 | 2.052 | 32.431 | 16.208 | 1.00 | 11.19 |
| 104 | N | ASN | A | 14 | 3.353 | 34.264 | 16.270 | 1.00 | 11.57 |
| 105 | CA | ASN | A | 14 | 4.546 | 33.500 | 15.961 | 1.00 | 11.48 |
| 106 | CB | ASN | A | 14 | 5.595 | 34.334 | 15.210 | 1.00 | 12.45 |
| 107 | CG | ASN | A | 14 | 6.194 | 35.434 | 16.051 | 1.00 | 13.41 |
| 108 | OD1 | ASN | A | 14 | 6.051 | 35.444 | 17.270 | 1.00 | 13.43 |
| 109 | ND2 | ASN | A | 14 | 6.893 | 36.360 | 15.399 | 1.00 | 14.05 |
| 110 | C | ASN | A | 14 | 5.131 | 32.866 | 17.200 | 1.00 | 13.19 |
| 111 | O | ASN | A | 14 | 4.574 | 32.987 | 18.287 | 1.00 | 13.86 |
| 112 | N | ILE | A | 15 | 6.245 | 32.172 | 17.031 | 1.00 | 15.08 |
| 113 | CA | ILE | A | 15 | 6.867 | 31.464 | 18.145 | 1.00 | 17.72 |
| 114 | CB | ILE | A | 15 | 8.140 | 30.720 | 17.669 | 1.00 | 18.28 |
| 115 | CG2 | ILE | A | 15 | 9.172 | 31.725 | 17.170 | 1.00 | 20.74 |
| 116 | CG1 | ILE | A | 15 | 8.684 | 29.838 | 18.789 | 1.00 | 23.00 |
| 117 | CD1 | ILE | A | 15 | 7.772 | 28.699 | 19.133 | 1.00 | 21.89 |
| 118 | C | ILE | A | 15 | 7.209 | 32.352 | 19.326 | 1.00 | 19.15 |
| 119 | O | ILE | A | 15 | 7.269 | 31.868 | 20.459 | 1.00 | 21.65 |
| 120 | N | ASN | A | 16 | 7.415 | 33.640 | 19.067 | 1.00 | 18.32 |
| 121 | CA | ASN | A | 16 | 7.758 | 34.584 | 20.130 | 1.00 | 18.96 |
| 122 | CB | ASN | A | 16 | 8.870 | 35.496 | 19.645 | 1.00 | 20.26 |
| 123 | CG | ASN | A | 16 | 10.149 | 34.725 | 19.382 | 1.00 | 23.71 |
| 124 | OD1 | ASN | A | 16 | 10.899 | 35.029 | 18.464 | 1.00 | 25.03 |
| 125 | ND2 | ASN | A | 16 | 10.393 | 33.703 | 20.200 | 1.00 | 24.94 |
| 126 | C | ASN | A | 16 | 6.557 | 35.372 | 20.620 | 1.00 | 18.55 |
| 127 | O | ASN | A | 16 | 6.696 | 36.409 | 21.267 | 1.00 | 20.18 |
| 128 | N | ASP | A | 17 | 5.374 | 34.864 | 20.285 | 1.00 | 15.80 |
| 129 | CA | ASP | A | 17 | 4.098 | 35.423 | 20.714 | 1.00 | 14.14 |
| 130 | CB | ASP | A | 17 | 4.084 | 35.544 | 22.247 | 1.00 | 19.26 |
| 131 | CG | AASP | A | 17 | 4.336 | 34.218 | 22.934 | 0.50 | 21.08 |
| 132 | CG | BASP | A | 17 | 2.745 | 35.196 | 22.839 | 0.50 | 19.49 |
| 133 | OD1 | AASP | A | 17 | 3.690 | 33.226 | 22.557 | 0.50 | 21.91 |
| 134 | OD1 | BASP | A | 17 | 1.982 | 34.434 | 22.201 | 0.50 | 22.80 |
| 135 | OD2 | AASP | A | 17 | 5.174 | 34.168 | 23.855 | 0.50 | 23.89 |
| 136 | OD2 | BASP | A | 17 | 2.469 | 35.663 | 23.966 | 0.50 | 24.36 |
| 137 | C | ASP | A | 17 | 3.715 | 36.761 | 20.086 | 1.00 | 14.47 |
| 138 | O | ASP | A | 17 | 2.844 | 37.477 | 20.585 | 1.00 | 16.52 |
| 139 | N | GLN | A | 18 | 4.380 | 37.086 | 18.992 | 1.00 | 13.41 |
| 140 | CA | GLN | A | 18 | 4.098 | 38.321 | 18.279 | 1.00 | 12.27 |
| 141 | CB | GLN | A | 18 | 5.314 | 38.749 | 17.483 | 1.00 | 12.96 |
| 142 | CG | GLN | A | 18 | 6.512 | 39.055 | 18.361 | 1.00 | 14.89 |
| 143 | CD | GLN | A | 18 | 7.732 | 39.329 | 17.565 | 1.00 | 16.33 |
| 144 | OE1 | GLN | A | 18 | 8.081 | 38.567 | 16.673 | 1.00 | 16.77 |
| 145 | NE2 | GLN | A | 18 | 8.413 | 40.438 | 17.880 | 1.00 | 19.49 |
| 146 | C | GLN | A | 18 | 2.917 | 38.095 | 17.358 | 1.00 | 13.10 |
| 147 | O | GLN | A | 18 | 2.763 | 37.027 | 16.772 | 1.00 | 12.70 |
| 148 | N | VAL | A | 19 | 2.096 | 39.126 | 17.220 | 1.00 | 12.22 |
| 149 | CA | VAL | A | 19 | 0.877 | 39.040 | 16.421 | 1.00 | 12.52 |
| 150 | CB | AVAL | A | 19 | −0.166 | 40.030 | 17.019 | 0.50 | 11.54 |
| 151 | CB | BVAL | A | 19 | −0.216 | 39.956 | 17.005 | 0.50 | 13.33 |
| 152 | CG1 | AVAL | A | 19 | −1.337 | 40.222 | 16.080 | 0.50 | 12.35 |
| 153 | CG1 | BVAL | A | 19 | 0.270 | 41.372 | 17.005 | 0.50 | 14.39 |
| 154 | CG2 | AVAL | A | 19 | −0.616 | 39.541 | 18.387 | 0.50 | 9.67 |
| 155 | CG2 | BVAL | A | 19 | −1.501 | 39.832 | 16.203 | 0.50 | 16.45 |
| 156 | C | VAL | A | 19 | 1.121 | 39.406 | 14.953 | 1.00 | 12.96 |
| 157 | O | VAL | A | 19 | 1.803 | 40.396 | 14.664 | 1.00 | 12.90 |
| 158 | N | LEU | A | 20 | 0.578 | 38.596 | 14.040 | 1.00 | 11.20 |
| 159 | CA | LEU | A | 20 | 0.674 | 38.894 | 12.607 | 1.00 | 9.98 |
| 160 | CB | LEU | A | 20 | 0.178 | 37.689 | 11.789 | 1.00 | 11.57 |
| 161 | CG | LEU | A | 20 | 0.179 | 37.923 | 10.290 | 1.00 | 11.32 |
| 162 | CD1 | LEU | A | 20 | 1.585 | 38.347 | 9.781 | 1.00 | 13.05 |
| 163 | CD2 | LEU | A | 20 | −0.234 | 36.609 | 9.671 | 1.00 | 12.15 |
| 164 | C | LEU | A | 20 | −0.237 | 40.119 | 12.397 | 1.00 | 12.64 |
| 165 | O | LEU | A | 20 | −1.417 | 40.097 | 12.714 | 1.00 | 11.62 |
| 166 | N | PHE | A | 21 | 0.340 | 41.177 | 11.845 | 1.00 | 13.85 |
| 167 | CA | PHE | A | 21 | −0.349 | 42.452 | 11.631 | 1.00 | 12.63 |
| 168 | CB | PHE | A | 21 | 0.342 | 43.505 | 12.500 | 1.00 | 13.71 |
| 169 | CG | PHE | A | 21 | −0.409 | 44.797 | 12.664 | 1.00 | 15.44 |
| 170 | CD1 | PHE | A | 21 | −0.460 | 45.735 | 11.632 | 1.00 | 18.36 |
| 171 | CD2 | PHE | A | 21 | −0.992 | 45.109 | 13.894 | 1.00 | 16.04 |
| 172 | CE1 | PHE | A | 21 | −1.078 | 46.976 | 11.824 | 1.00 | 19.58 |
| 173 | CE2 | PHE | A | 21 | −1.619 | 46.347 | 14.102 | 1.00 | 18.12 |
| 174 | CZ | PHE | A | 21 | −1.664 | 47.284 | 13.068 | 1.00 | 16.49 |
| 175 | C | PHE | A | 21 | −0.167 | 42.772 | 10.160 | 1.00 | 12.86 |
| 176 | O | PHE | A | 21 | 0.949 | 43.059 | 9.732 | 1.00 | 15.40 |
| 177 | N | VAL | A | 22 | −1.254 | 42.720 | 9.388 | 1.00 | 12.83 |
| 178 | CA | VAL | A | 22 | −1.175 | 42.969 | 7.945 | 1.00 | 13.23 |
| 179 | CB | VAL | A | 22 | −1.914 | 41.861 | 7.160 | 1.00 | 11.71 |
| 180 | CG1 | VAL | A | 22 | −1.639 | 42.007 | 5.668 | 1.00 | 11.83 |
| 181 | CG2 | VAL | A | 22 | −1.501 | 40.470 | 7.664 | 1.00 | 12.72 |
| 182 | C | VAL | A | 22 | −1.825 | 44.310 | 7.648 | 1.00 | 13.64 |
| 183 | O | VAL | A | 22 | −3.051 | 44.409 | 7.588 | 1.00 | 13.52 |
| 184 | N | ASP | A | 23 | −1.003 | 45.339 | 7.463 | 1.00 | 15.46 |
| 185 | CA | ASP | A | 23 | −1.508 | 46.684 | 7.216 | 1.00 | 15.25 |
| 186 | CB | ASP | A | 23 | −0.786 | 47.654 | 8.163 | 1.00 | 16.03 |
| 187 | CG | ASP | A | 23 | −1.477 | 48.994 | 8.286 | 1.00 | 21.45 |
| 188 | OD1 | ASP | A | 23 | −2.623 | 49.149 | 7.822 | 1.00 | 20.42 |
| 189 | OD2 | ASP | A | 23 | −0.849 | 49.898 | 8.876 | 1.00 | 23.14 |
| 190 | C | ASP | A | 23 | −1.242 | 47.044 | 5.758 | 1.00 | 16.45 |
| 191 | O | ASP | A | 23 | −0.096 | 47.019 | 5.312 | 1.00 | 16.34 |
| 192 | N | LYS | A | 24 | −2.313 | 47.372 | 5.045 | 1.00 | 18.40 |
| 193 | CA | LYS | A | 24 | −2.237 | 47.711 | 3.624 | 1.00 | 20.51 |
| 194 | CB | LYS | A | 24 | −1.612 | 49.091 | 3.417 | 1.00 | 23.94 |
| 195 | CG | LYS | A | 24 | −2.485 | 50.255 | 3.888 | 1.00 | 27.86 |
| 196 | CD | ALYS | A | 24 | −2.967 | 50.108 | 5.314 | 0.50 | 27.38 |
| 197 | CD | BLYS | A | 24 | −1.724 | 51.580 | 3.810 | 0.50 | 28.39 |
| 198 | CE | ALYS | A | 24 | −3.823 | 51.299 | 5.739 | 0.50 | 27.92 |
| 199 | CE | BLYS | A | 24 | −2.476 | 52.710 | 4.509 | 0.50 | 28.38 |
| 200 | NZ | ALYS | A | 24 | −4.368 | 51.137 | 7.117 | 0.50 | 24.39 |
| 201 | NZ | BLYS | A | 24 | −1.702 | 53.988 | 4.536 | 0.50 | 30.10 |
| 202 | C | LYS | A | 24 | −1.418 | 46.629 | 2.935 | 1.00 | 20.07 |
| 203 | O | LYS | A | 24 | −0.419 | 46.891 | 2.246 | 1.00 | 21.56 |
| 204 | N | ARG | A | 25 | −1.845 | 45.397 | 3.195 | 1.00 | 19.53 |
| 205 | CA | ARG | A | 25 | −1.271 | 44.185 | 2.620 | 1.00 | 17.88 |
| 206 | CB | ARG | A | 25 | −1.501 | 44.223 | 1.103 | 1.00 | 20.64 |
| 207 | CG | ARG | A | 25 | −2.965 | 44.456 | 0.781 | 1.00 | 21.58 |
| 208 | CD | ARG | A | 25 | −3.298 | 44.248 | −0.692 | 1.00 | 26.78 |
| 209 | NE | ARG | A | 25 | −2.751 | 45.280 | −1.560 | 1.00 | 31.19 |
| 210 | CZ | ARG | A | 25 | −1.867 | 45.053 | −2.530 | 1.00 | 35.33 |
| 211 | NH1 | ARG | A | 25 | −1.417 | 43.826 | −2.753 | 1.00 | 34.83 |
| 212 | NH2 | ARG | A | 25 | −1.452 | 46.054 | −3.299 | 1.00 | 35.49 |
| 213 | C | ARG | A | 25 | 0.183 | 43.858 | 2.951 | 1.00 | 17.97 |
| 214 | O | ARG | A | 25 | 0.772 | 42.946 | 2.369 | 1.00 | 17.05 |
| 215 | N | GLN | A | 26 | 0.766 | 44.577 | 3.905 | 1.00 | 16.87 |
| 216 | CA | GLN | A | 26 | 2.148 | 44.318 | 4.287 | 1.00 | 16.96 |
| 217 | CB | GLN | A | 26 | 2.912 | 45.631 | 4.485 | 1.00 | 20.86 |
| 218 | CG | GLN | A | 26 | 2.983 | 46.507 | 3.247 | 1.00 | 23.99 |
| 219 | CD | GLN | A | 26 | 3.629 | 45.794 | 2.089 | 1.00 | 25.42 |
| 220 | OE1 | GLN | A | 26 | 4.611 | 45.067 | 2.264 | 1.00 | 29.88 |
| 221 | NE2 | GLN | A | 26 | 3.092 | 45.998 | 0.905 | 1.00 | 27.56 |
| 222 | C | GLN | A | 26 | 2.119 | 43.567 | 5.610 | 1.00 | 17.72 |
| 223 | O | GLN | A | 26 | 1.723 | 44.128 | 6.629 | 1.00 | 18.68 |
| 224 | N | PRO | A | 27 | 2.528 | 42.296 | 5.606 | 1.00 | 16.31 |
| 225 | CD | PRO | A | 27 | 2.886 | 41.451 | 4.455 | 1.00 | 16.37 |
| 226 | CA | PRO | A | 27 | 2.519 | 41.519 | 6.845 | 1.00 | 17.06 |
| 227 | CB | PRO | A | 27 | 2.374 | 40.104 | 6.343 | 1.00 | 16.90 |
| 228 | CG | PRO | A | 27 | 3.228 | 40.131 | 5.106 | 1.00 | 17.58 |
| 229 | C | PRO | A | 27 | 3.786 | 41.694 | 7.652 | 1.00 | 18.72 |
| 230 | O | PRO | A | 27 | 4.884 | 41.746 | 7.096 | 1.00 | 22.96 |
| 231 | N | VAL | A | 28 | 3.629 | 41.832 | 8.962 | 1.00 | 15.03 |
| 232 | CA | VAL | A | 28 | 4.761 | 41.927 | 9.860 | 1.00 | 14.94 |
| 233 | CB | VAL | A | 28 | 5.211 | 43.383 | 10.078 | 1.00 | 16.40 |
| 234 | CG1 | VAL | A | 28 | 5.445 | 44.053 | 8.721 | 1.00 | 20.59 |
| 235 | CG2 | VAL | A | 28 | 4.202 | 44.144 | 10.876 | 1.00 | 18.71 |
| 236 | C | VAL | A | 28 | 4.267 | 41.355 | 11.182 | 1.00 | 14.92 |
| 237 | O | VAL | A | 28 | 3.059 | 41.221 | 11.390 | 1.00 | 15.52 |
| 238 | N | PHE | A | 29 | 5.188 | 40.971 | 12.056 | 1.00 | 13.35 |
| 239 | CA | PHE | A | 29 | 4.786 | 40.494 | 13.369 | 1.00 | 13.87 |
| 240 | CB | PHE | A | 29 | 5.525 | 39.216 | 13.717 | 1.00 | 13.00 |
| 241 | CG | PHE | A | 29 | 5.075 | 38.042 | 12.917 | 1.00 | 11.79 |
| 242 | CD1 | PHE | A | 29 | 3.991 | 37.260 | 13.347 | 1.00 | 13.09 |
| 243 | CD2 | PHE | A | 29 | 5.711 | 37.729 | 11.719 | 1.00 | 12.43 |
| 244 | CE1 | PHE | A | 29 | 3.545 | 36.178 | 12.583 | 1.00 | 12.68 |
| 245 | CE2 | PHE | A | 29 | 5.273 | 36.647 | 10.956 | 1.00 | 13.02 |
| 246 | CZ | PHE | A | 29 | 4.183 | 35.869 | 11.388 | 1.00 | 12.83 |
| 247 | C | PHE | A | 29 | 5.115 | 41.589 | 14.369 | 1.00 | 12.75 |
| 248 | O | PHE | A | 29 | 6.179 | 42.215 | 14.275 | 1.00 | 15.29 |
| 249 | N | GLU | A | 30 | 4.203 | 41.817 | 15.309 | 1.00 | 13.51 |
| 250 | CA | GLU | A | 30 | 4.351 | 42.861 | 16.327 | 1.00 | 14.32 |
| 251 | CB | GLU | A | 30 | 3.262 | 43.907 | 16.152 | 1.00 | 18.01 |
| 252 | CG | GLU | A | 30 | 3.320 | 44.616 | 14.859 | 1.00 | 20.70 |
| 253 | CD | GLU | A | 30 | 4.230 | 45.806 | 14.942 | 1.00 | 23.57 |
| 254 | OE1 | GLU | A | 30 | 5.034 | 45.885 | 15.906 | 1.00 | 26.17 |
| 255 | OE2 | GLU | A | 30 | 4.148 | 46.662 | 14.050 | 1.00 | 26.38 |
| 256 | C | GLU | A | 30 | 4.200 | 42.356 | 17.731 | 1.00 | 15.20 |
| 257 | O | GLU | A | 30 | 3.391 | 41.479 | 17.980 | 1.00 | 15.87 |
| 258 | N | ASP | A | 31 | 4.946 | 42.931 | 18.667 | 1.00 | 16.69 |
| 259 | CA | ASP | A | 31 | 4.777 | 42.540 | 20.049 | 1.00 | 17.47 |
| 260 | CB | ASP | A | 31 | 5.833 | 43.182 | 20.954 | 1.00 | 20.86 |
| 261 | CG | ASP | A | 31 | 7.232 | 42.715 | 20.636 | 1.00 | 25.47 |
| 262 | OD1 | ASP | A | 31 | 7.419 | 41.517 | 20.337 | 1.00 | 26.85 |
| 263 | OD2 | ASP | A | 31 | 8.159 | 43.546 | 20.704 | 1.00 | 33.31 |
| 264 | C | ASP | A | 31 | 3.423 | 43.079 | 20.427 | 1.00 | 15.46 |
| 265 | O | ASP | A | 31 | 3.058 | 44.197 | 20.041 | 1.00 | 15.35 |
| 266 | N | MET | A | 32 | 2.674 | 42.296 | 21.181 | 1.00 | 14.56 |
| 267 | CA | MET | A | 32 | 1.345 | 42.681 | 21.610 | 1.00 | 15.04 |
| 268 | CB | MET | A | 32 | 0.755 | 41.574 | 22.466 | 1.00 | 19.75 |
| 269 | CG | MET | A | 32 | 0.302 | 40.406 | 21.633 | 1.00 | 22.06 |
| 270 | SD | MET | A | 32 | −0.501 | 39.183 | 22.659 | 1.00 | 29.43 |
| 271 | CE | MET | A | 32 | 0.859 | 38.149 | 22.940 | 1.00 | 27.59 |
| 272 | C | MET | A | 32 | 1.331 | 43.988 | 22.384 | 1.00 | 15.28 |
| 273 | O | MET | A | 32 | 0.398 | 44.758 | 22.276 | 1.00 | 19.59 |
| 274 | N | THR | A | 33 | 2.377 | 44.221 | 23.162 | 1.00 | 16.47 |
| 275 | CA | THR | A | 33 | 2.437 | 45.443 | 23.960 | 1.00 | 16.94 |
| 276 | CB | THR | A | 33 | 3.638 | 45.450 | 24.942 | 1.00 | 14.34 |
| 277 | OG1 | THR | A | 33 | 4.846 | 45.277 | 24.207 | 1.00 | 17.85 |
| 278 | CG2 | THR | A | 33 | 3.518 | 44.350 | 25.983 | 1.00 | 19.59 |
| 279 | C | THR | A | 33 | 2.574 | 46.696 | 23.115 | 1.00 | 17.33 |
| 280 | O | THR | A | 33 | 2.253 | 47.797 | 23.592 | 1.00 | 18.77 |
| 281 | N | ASP | A | 34 | 3.034 | 46.532 | 21.874 | 1.00 | 16.12 |
| 282 | CA | ASP | A | 34 | 3.284 | 47.667 | 20.993 | 1.00 | 16.46 |
| 283 | CB | ASP | A | 34 | 4.489 | 47.386 | 20.107 | 1.00 | 18.70 |
| 284 | CG | ASP | A | 34 | 5.755 | 47.178 | 20.907 | 1.00 | 21.90 |
| 285 | OD1 | ASP | A | 34 | 5.709 | 47.403 | 22.134 | 1.00 | 26.20 |
| 286 | OD2 | ASP | A | 34 | 6.782 | 46.800 | 20.307 | 1.00 | 26.01 |
| 287 | C | ASP | A | 34 | 2.154 | 48.106 | 20.115 | 1.00 | 16.97 |
| 288 | O | ASP | A | 34 | 2.267 | 49.132 | 19.458 | 1.00 | 17.02 |
| 289 | N | ILE | A | 35 | 1.077 | 47.342 | 20.097 | 1.00 | 15.54 |
| 290 | CA | ILE | A | 35 | −0.065 | 47.694 | 19.264 | 1.00 | 14.41 |
| 291 | CB | ILE | A | 35 | −0.300 | 46.627 | 18.156 | 1.00 | 17.77 |
| 292 | CG2 | ILE | A | 35 | 0.908 | 46.541 | 17.234 | 1.00 | 18.36 |
| 293 | CG1 | ILE | A | 35 | −0.623 | 45.273 | 18.806 | 1.00 | 17.07 |
| 294 | CD1 | ILE | A | 35 | −1.004 | 44.179 | 17.795 | 1.00 | 19.32 |
| 295 | C | ILE | A | 35 | −1.340 | 47.745 | 20.084 | 1.00 | 16.21 |
| 296 | O | ILE | A | 35 | −1.392 | 47.254 | 21.214 | 1.00 | 15.80 |
| 297 | N | ASP | A | 36 | −2.373 | 48.350 | 19.520 | 1.00 | 14.90 |
| 298 | CA | ASP | A | 36 | −3.677 | 48.332 | 20.151 | 1.00 | 16.77 |
| 299 | CB | ASP | A | 36 | −4.351 | 49.700 | 20.103 | 1.00 | 16.16 |
| 300 | CG | ASP | A | 36 | −5.771 | 49.647 | 20.617 | 1.00 | 19.51 |
| 301 | OD1 | ASP | A | 36 | −6.328 | 48.531 | 20.692 | 1.00 | 20.01 |
| 302 | OD2 | ASP | A | 36 | −6.347 | 50.704 | 20.934 | 1.00 | 18.76 |
| 303 | C | ASP | A | 36 | −4.428 | 47.351 | 19.247 | 1.00 | 16.91 |
| 304 | O | ASP | A | 36 | −4.926 | 47.720 | 18.172 | 1.00 | 17.46 |
| 305 | N | GLN | A | 37 | −4.503 | 46.095 | 19.663 | 1.00 | 20.31 |
| 306 | CA | GLN | A | 37 | −5.158 | 45.105 | 18.831 | 1.00 | 19.91 |
| 307 | CB | GLN | A | 37 | −5.006 | 43.721 | 19.445 | 1.00 | 21.24 |
| 308 | CG | GLN | A | 37 | −5.470 | 42.599 | 18.543 | 1.00 | 24.10 |
| 309 | CD | GLN | A | 37 | −5.389 | 41.265 | 19.256 | 1.00 | 24.17 |
| 310 | OE1 | GLN | A | 37 | −4.324 | 40.874 | 19.741 | 1.00 | 26.60 |
| 311 | NE2 | GLN | A | 37 | −6.514 | 40.567 | 19.332 | 1.00 | 25.48 |
| 312 | C | GLN | A | 37 | −6.625 | 45.386 | 18.567 | 1.00 | 19.67 |
| 313 | O | GLN | A | 37 | −7.129 | 45.130 | 17.465 | 1.00 | 20.86 |
| 314 | N | SER | A | 38 | −7.326 | 45.924 | 19.559 | 1.00 | 19.52 |
| 315 | CA | SER | A | 38 | −8.738 | 46.210 | 19.359 | 1.00 | 20.61 |
| 316 | CB | SER | A | 38 | −9.375 | 46.654 | 20.679 | 1.00 | 23.23 |
| 317 | OG | SER | A | 38 | −8.960 | 47.968 | 21.022 | 1.00 | 26.19 |
| 318 | C | SER | A | 38 | −8.967 | 47.266 | 18.281 | 1.00 | 19.57 |
| 319 | O | SER | A | 38 | −10.029 | 47.308 | 17.665 | 1.00 | 21.18 |
| 320 | N | ALA | A | 39 | −7.961 | 48.107 | 18.031 | 1.00 | 18.48 |
| 321 | CA | ALA | A | 39 | −8.094 | 49.155 | 17.023 | 1.00 | 17.27 |
| 322 | CB | ALA | A | 39 | −7.388 | 50.428 | 17.488 | 1.00 | 16.51 |
| 323 | C | ALA | A | 39 | −7.499 | 48.704 | 15.702 | 1.00 | 18.81 |
| 324 | O | ALA | A | 39 | −7.425 | 49.471 | 14.753 | 1.00 | 19.79 |
| 325 | N | SER | A | 40 | −7.072 | 47.450 | 15.668 | 1.00 | 18.59 |
| 326 | CA | SER | A | 40 | −6.424 | 46.887 | 14.499 | 1.00 | 18.28 |
| 327 | CB | SER | A | 40 | −4.967 | 46.623 | 14.839 | 1.00 | 20.22 |
| 328 | OG | SER | A | 40 | −4.368 | 47.772 | 15.419 | 1.00 | 18.89 |
| 329 | C | SER | A | 40 | −7.060 | 45.585 | 14.042 | 1.00 | 18.60 |
| 330 | O | SER | A | 40 | −6.343 | 44.686 | 13.614 | 1.00 | 16.43 |
| 331 | N | GLU | A | 41 | −8.383 | 45.490 | 14.127 | 1.00 | 18.44 |
| 332 | CA | GLU | A | 41 | −9.087 | 44.268 | 13.735 | 1.00 | 19.57 |
| 333 | CB | GLU | A | 41 | −10.594 | 44.442 | 13.990 | 1.00 | 21.67 |
| 334 | CG | GLU | A | 41 | −11.469 | 43.215 | 13.708 | 1.00 | 26.29 |
| 335 | CD | GLU | A | 41 | −11.811 | 43.001 | 12.219 | 1.00 | 29.17 |
| 336 | OE1 | GLU | A | 41 | −11.880 | 43.974 | 11.420 | 1.00 | 30.03 |
| 337 | OE2 | GLU | A | 41 | −12.037 | 41.831 | 11.850 | 1.00 | 31.07 |
| 338 | C | GLU | A | 41 | −8.856 | 43.812 | 12.299 | 1.00 | 18.24 |
| 339 | O | GLU | A | 41 | −8.517 | 42.641 | 12.064 | 1.00 | 16.26 |
| 340 | N | PRO | A | 42 | −9.022 | 44.709 | 11.313 | 1.00 | 18.25 |
| 341 | CD | PRO | A | 42 | −9.497 | 46.104 | 11.388 | 1.00 | 19.11 |
| 342 | CA | PRO | A | 42 | −8.815 | 44.313 | 9.915 | 1.00 | 18.20 |
| 343 | CB | PRO | A | 42 | −9.054 | 45.612 | 9.145 | 1.00 | 20.60 |
| 344 | CG | PRO | A | 42 | −10.075 | 46.308 | 9.984 | 1.00 | 19.63 |
| 345 | C | PRO | A | 42 | −7.418 | 43.783 | 9.649 | 1.00 | 18.52 |
| 346 | O | PRO | A | 42 | −7.215 | 42.941 | 8.771 | 1.00 | 20.18 |
| 347 | N | GLN | A | 43 | −6.459 | 44.278 | 10.421 | 1.00 | 16.41 |
| 348 | CA | GLN | A | 43 | −5.061 | 43.911 | 10.243 | 1.00 | 14.34 |
| 349 | CB | GLN | A | 43 | −4.150 | 45.062 | 10.682 | 1.00 | 15.38 |
| 350 | CG | GLN | A | 43 | −4.308 | 46.351 | 9.895 | 1.00 | 15.32 |
| 351 | CD | GLN | A | 43 | −5.633 | 47.029 | 10.107 | 1.00 | 16.71 |
| 352 | OE1 | GLN | A | 43 | −6.227 | 46.916 | 11.172 | 1.00 | 17.99 |
| 353 | NE2 | GLN | A | 43 | −6.096 | 47.764 | 9.107 | 1.00 | 18.35 |
| 354 | C | GLN | A | 43 | −4.609 | 42.673 | 10.996 | 1.00 | 13.64 |
| 355 | O | GLN | A | 43 | −3.582 | 42.110 | 10.671 | 1.00 | 13.77 |
| 356 | N | THR | A | 44 | −5.362 | 42.278 | 12.015 | 1.00 | 11.95 |
| 357 | CA | THR | A | 44 | −4.967 | 41.137 | 12.836 | 1.00 | 12.27 |
| 358 | CB | THR | A | 44 | −4.718 | 41.558 | 14.305 | 1.00 | 13.41 |
| 359 | OG1 | THR | A | 44 | −5.899 | 42.144 | 14.865 | 1.00 | 15.65 |
| 360 | CG2 | THR | A | 44 | −3.568 | 42.546 | 14.366 | 1.00 | 14.79 |
| 361 | C | THR | A | 44 | −5.922 | 39.964 | 12.848 | 1.00 | 12.84 |
| 362 | O | THR | A | 44 | −5.547 | 38.861 | 13.248 | 1.00 | 12.74 |
| 363 | N | ARG | A | 45 | −7.160 | 40.189 | 12.425 | 1.00 | 13.15 |
| 364 | CA | ARG | A | 45 | −8.113 | 39.098 | 12.416 | 1.00 | 16.60 |
| 365 | CB | ARG | A | 45 | −9.502 | 39.629 | 12.739 | 1.00 | 18.42 |
| 366 | CG | ARG | A | 45 | −10.545 | 38.550 | 12.897 | 1.00 | 24.87 |
| 367 | CD | ARG | A | 45 | −11.814 | 39.145 | 13.480 | 1.00 | 26.48 |
| 368 | NE | ARG | A | 45 | −11.617 | 39.596 | 14.855 | 1.00 | 29.29 |
| 369 | CZ | ARG | A | 45 | −12.495 | 40.327 | 15.545 | 1.00 | 29.33 |
| 370 | NH1 | ARG | A | 45 | −13.637 | 40.705 | 14.983 | 1.00 | 29.91 |
| 371 | NH2 | ARG | A | 45 | −12.247 | 40.651 | 16.811 | 1.00 | 27.73 |
| 372 | C | ARG | A | 45 | −8.066 | 38.458 | 11.042 | 1.00 | 16.09 |
| 373 | O | ARG | A | 45 | −8.691 | 38.932 | 10.097 | 1.00 | 18.98 |
| 374 | N | LEU | A | 46 | −7.294 | 37.395 | 10.926 | 1.00 | 13.35 |
| 375 | CA | LEU | A | 46 | −7.138 | 36.694 | 9.659 | 1.00 | 15.38 |
| 376 | CB | LEU | A | 46 | −5.919 | 35.765 | 9.726 | 1.00 | 16.55 |
| 377 | CG | LEU | A | 46 | −4.529 | 36.358 | 9.438 | 1.00 | 18.84 |
| 378 | CD1 | LEU | A | 46 | −4.313 | 36.464 | 7.954 | 1.00 | 19.37 |
| 379 | CD2 | LEU | A | 46 | −4.381 | 37.731 | 10.076 | 1.00 | 16.64 |
| 380 | C | LEU | A | 46 | −8.381 | 35.875 | 9.389 | 1.00 | 15.61 |
| 381 | O | LEU | A | 46 | −8.950 | 35.272 | 10.292 | 1.00 | 16.43 |
| 382 | N | ILE | A | 47 | −8.803 | 35.869 | 8.128 | 1.00 | 14.86 |
| 383 | CA | ILE | A | 47 | −9.958 | 35.101 | 7.706 | 1.00 | 14.41 |
| 384 | CB | ILE | A | 47 | −10.788 | 35.897 | 6.685 | 1.00 | 15.54 |
| 385 | CG2 | ILE | A | 47 | −11.880 | 35.029 | 6.102 | 1.00 | 15.91 |
| 386 | CG1 | ILE | A | 47 | −11.376 | 37.130 | 7.398 | 1.00 | 18.38 |
| 387 | CD1 | ILE | A | 47 | −11.678 | 38.302 | 6.515 | 1.00 | 22.09 |
| 388 | C | ILE | A | 47 | −9.392 | 33.828 | 7.084 | 1.00 | 14.94 |
| 389 | O | ILE | A | 47 | −8.687 | 33.874 | 6.073 | 1.00 | 16.17 |
| 390 | N | ILE | A | 48 | −9.687 | 32.699 | 7.705 | 1.00 | 13.61 |
| 391 | CA | ILE | A | 48 | −9.159 | 31.440 | 7.226 | 1.00 | 12.55 |
| 392 | CB | ILE | A | 48 | −8.475 | 30.699 | 8.373 | 1.00 | 14.43 |
| 393 | CG2 | ILE | A | 48 | −7.960 | 29.350 | 7.893 | 1.00 | 16.30 |
| 394 | CG1 | ILE | A | 48 | −7.367 | 31.606 | 8.953 | 1.00 | 17.50 |
| 395 | CD1 | ILE | A | 48 | −6.592 | 30.997 | 10.062 | 1.00 | 22.06 |
| 396 | C | ILE | A | 48 | −10.241 | 30.571 | 6.628 | 1.00 | 13.80 |
| 397 | O | ILE | A | 48 | −11.134 | 30.099 | 7.338 | 1.00 | 15.75 |
| 398 | N | TYR | A | 49 | −10.178 | 30.393 | 5.317 | 1.00 | 13.63 |
| 399 | CA | TYR | A | 49 | −11.151 | 29.537 | 4.653 | 1.00 | 13.80 |
| 400 | CB | TYR | A | 49 | −11.479 | 30.033 | 3.235 | 1.00 | 15.93 |
| 401 | CG | TYR | A | 49 | −12.604 | 31.032 | 3.180 | 1.00 | 16.52 |
| 402 | CD1 | TYR | A | 49 | −13.934 | 30.610 | 3.114 | 1.00 | 18.44 |
| 403 | CE1 | TYR | A | 49 | −14.972 | 31.533 | 3.031 | 1.00 | 19.03 |
| 404 | CD2 | TYR | A | 49 | −12.335 | 32.397 | 3.172 | 1.00 | 16.27 |
| 405 | CE2 | TYR | A | 49 | −13.377 | 33.339 | 3.092 | 1.00 | 19.45 |
| 406 | CZ | TYR | A | 49 | −14.688 | 32.891 | 3.019 | 1.00 | 20.98 |
| 407 | OH | TYR | A | 49 | −15.716 | 33.813 | 2.935 | 1.00 | 21.87 |
| 408 | C | TYR | A | 49 | −10.604 | 28.134 | 4.513 | 1.00 | 14.64 |
| 409 | O | TYR | A | 49 | −9.444 | 27.935 | 4.116 | 1.00 | 13.90 |
| 410 | N | MET | A | 50 | −11.443 | 27.170 | 4.855 | 1.00 | 15.66 |
| 411 | CA | MET | A | 50 | −11.123 | 25.765 | 4.698 | 1.00 | 15.50 |
| 412 | CB | MET | A | 50 | −11.975 | 24.921 | 5.636 | 1.00 | 18.56 |
| 413 | CG | MET | A | 50 | −11.541 | 24.956 | 7.077 | 1.00 | 21.58 |
| 414 | SD | MET | A | 50 | −12.663 | 23.992 | 8.081 | 1.00 | 27.12 |
| 415 | CE | MET | A | 50 | −12.298 | 22.355 | 7.561 | 1.00 | 22.95 |
| 416 | C | MET | A | 50 | −11.508 | 25.400 | 3.266 | 1.00 | 17.81 |
| 417 | O | MET | A | 50 | −12.481 | 25.931 | 2.721 | 1.00 | 18.06 |
| 418 | N | TYR | A | 51 | −10.750 | 24.502 | 2.658 | 1.00 | 15.81 |
| 419 | CA | TYR | A | 51 | −11.045 | 24.035 | 1.303 | 1.00 | 15.35 |
| 420 | CB | TYR | A | 51 | −9.964 | 24.465 | 0.295 | 1.00 | 14.24 |
| 421 | CG | TYR | A | 51 | −10.075 | 25.894 | −0.177 | 1.00 | 14.38 |
| 422 | CD1 | TYR | A | 51 | −9.771 | 26.957 | 0.665 | 1.00 | 14.26 |
| 423 | CE1 | TYR | A | 51 | −9.907 | 28.274 | 0.242 | 1.00 | 15.84 |
| 424 | CD2 | TYR | A | 51 | −10.517 | 26.189 | −1.473 | 1.00 | 13.13 |
| 425 | CE2 | TYR | A | 51 | −10.655 | 27.505 | −1.917 | 1.00 | 14.07 |
| 426 | CZ | TYR | A | 51 | −10.347 | 28.553 | −1.044 | 1.00 | 14.00 |
| 427 | OH | TYR | A | 51 | −10.464 | 29.843 | −1.493 | 1.00 | 15.60 |
| 428 | C | TYR | A | 51 | −11.065 | 22.519 | 1.300 | 1.00 | 18.24 |
| 429 | O | TYR | A | 51 | −10.354 | 21.878 | 2.080 | 1.00 | 18.55 |
| 430 | N | LYS | A | 52 | −11.896 | 21.964 | 0.425 | 1.00 | 18.07 |
| 431 | CA | LYS | A | 52 | −11.943 | 20.534 | 0.220 | 1.00 | 18.12 |
| 432 | CB | LYS | A | 52 | −13.300 | 20.099 | −0.359 | 1.00 | 20.55 |
| 433 | CG | LYS | A | 52 | −13.261 | 18.680 | −0.935 | 1.00 | 23.64 |
| 434 | CD | LYS | A | 52 | −14.592 | 18.298 | −1.534 | 1.00 | 24.82 |
| 435 | CE | LYS | A | 52 | −14.548 | 16.879 | −2.067 | 1.00 | 28.40 |
| 436 | NZ | LYS | A | 52 | −13.634 | 16.776 | −3.222 | 1.00 | 31.95 |
| 437 | C | LYS | A | 52 | −10.848 | 20.302 | −0.825 | 1.00 | 18.51 |
| 438 | O | LYS | A | 52 | −10.754 | 21.023 | −1.818 | 1.00 | 19.93 |
| 439 | N | ASP | A | 53 | −10.010 | 19.300 | −0.588 | 1.00 | 17.99 |
| 440 | CA | ASP | A | 53 | −8.912 | 18.963 | −1.490 | 1.00 | 14.95 |
| 441 | CB | ASP | A | 53 | −7.618 | 18.868 | −0.677 | 1.00 | 15.86 |
| 442 | CG | ASP | A | 53 | −6.429 | 18.361 | −1.477 | 1.00 | 13.89 |
| 443 | OD1 | ASP | A | 53 | −6.400 | 18.411 | −2.730 | 1.00 | 14.82 |
| 444 | OD2 | ASP | A | 53 | −5.459 | 17.912 | −0.838 | 1.00 | 15.61 |
| 445 | C | ASP | A | 53 | −9.214 | 17.623 | −2.143 | 1.00 | 16.55 |
| 446 | O | ASP | A | 53 | −9.422 | 16.638 | −1.446 | 1.00 | 18.70 |
| 447 | N | SER | A | 54 | −9.244 | 17.589 | −3.473 | 1.00 | 17.52 |
| 448 | CA | SER | A | 54 | −9.535 | 16.342 | −4.163 | 1.00 | 18.31 |
| 449 | CB | SER | A | 54 | −9.570 | 16.561 | −5.678 | 1.00 | 14.96 |
| 450 | OG | SER | A | 54 | −8.297 | 16.938 | −6.182 | 1.00 | 19.31 |
| 451 | C | SER | A | 54 | −8.518 | 15.273 | −3.810 | 1.00 | 18.78 |
| 452 | O | SER | A | 54 | −8.845 | 14.080 | −3.816 | 1.00 | 21.77 |
| 453 | N | GLU | A | 55 | −7.302 | 15.692 | −3.481 | 1.00 | 17.60 |
| 454 | CA | GLU | A | 55 | −6.253 | 14.752 | −3.130 | 1.00 | 18.32 |
| 455 | CB | GLU | A | 55 | −4.880 | 15.300 | −3.523 | 1.00 | 22.79 |
| 456 | CG | GLU | A | 55 | −4.596 | 15.218 | −5.008 | 1.00 | 28.65 |
| 457 | CD | GLU | A | 55 | −4.652 | 13.784 | −5.491 | 1.00 | 32.26 |
| 458 | OE1 | GLU | A | 55 | −5.548 | 13.478 | −6.307 | 1.00 | 34.10 |
| 459 | OE2 | GLU | A | 55 | −3.812 | 12.964 | −5.034 | 1.00 | 35.46 |
| 460 | C | GLU | A | 55 | −6.256 | 14.403 | −1.656 | 1.00 | 17.26 |
| 461 | O | GLU | A | 55 | −5.428 | 13.619 | −1.237 | 1.00 | 19.07 |
| 462 | N | VAL | A | 56 | −7.187 | 14.973 | −0.882 | 1.00 | 17.06 |
| 463 | CA | VAL | A | 56 | −7.297 | 14.696 | 0.553 | 1.00 | 16.58 |
| 464 | CB | AVAL | A | 56 | −7.984 | 13.341 | 0.804 | 0.50 | 17.64 |
| 465 | CB | BVAL | A | 56 | −7.962 | 13.322 | 0.796 | 0.50 | 15.94 |
| 466 | CG1 | AVAL | A | 56 | −9.322 | 13.290 | 0.088 | 0.50 | 17.39 |
| 467 | CG1 | BVAL | A | 56 | −8.523 | 13.266 | 2.213 | 0.50 | 14.65 |
| 468 | CG2 | AVAL | A | 56 | −7.111 | 12.237 | 0.336 | 0.50 | 19.35 |
| 469 | CG2 | BVAL | A | 56 | −9.067 | 13.083 | −0.231 | 0.50 | 14.51 |
| 470 | C | VAL | A | 56 | −5.926 | 14.712 | 1.249 | 1.00 | 17.26 |
| 471 | O | VAL | A | 56 | −5.538 | 13.771 | 1.957 | 1.00 | 18.42 |
| 472 | N | ARG | A | 57 | −5.178 | 15.791 | 1.041 | 1.00 | 15.87 |
| 473 | CA | ARG | A | 57 | −3.853 | 15.911 | 1.646 | 1.00 | 15.65 |
| 474 | CB | ARG | A | 57 | −2.925 | 16.717 | 0.748 | 1.00 | 17.60 |
| 475 | CG | ARG | A | 57 | −2.380 | 15.940 | −0.414 | 1.00 | 19.60 |
| 476 | CD | ARG | A | 57 | −1.780 | 16.874 | −1.422 | 1.00 | 19.85 |
| 477 | NE | ARG | A | 57 | −2.848 | 17.626 | −2.066 | 1.00 | 23.12 |
| 478 | CZ | ARG | A | 57 | −2.672 | 18.496 | −3.049 | 1.00 | 26.23 |
| 479 | NH1 | ARG | A | 57 | −1.456 | 18.749 | −3.511 | 1.00 | 23.53 |
| 480 | NH2 | ARG | A | 57 | −3.730 | 19.077 | −3.599 | 1.00 | 22.31 |
| 481 | C | ARG | A | 57 | −3.886 | 16.541 | 3.020 | 1.00 | 16.15 |
| 482 | O | ARG | A | 57 | −2.849 | 16.649 | 3.673 | 1.00 | 18.57 |
| 483 | N | GLY | A | 58 | −5.068 | 16.951 | 3.460 | 1.00 | 14.70 |
| 484 | CA | GLY | A | 58 | −5.183 | 17.544 | 4.779 | 1.00 | 17.17 |
| 485 | C | GLY | A | 58 | −6.057 | 18.784 | 4.898 | 1.00 | 17.08 |
| 486 | O | GLY | A | 58 | −7.023 | 18.997 | 4.143 | 1.00 | 17.15 |
| 487 | N | LEU | A | 59 | −5.712 | 19.617 | 5.873 | 1.00 | 15.26 |
| 488 | CA | LEU | A | 59 | −6.477 | 20.816 | 6.153 | 1.00 | 16.29 |
| 489 | CB | LEU | A | 59 | −6.341 | 21.198 | 7.629 | 1.00 | 21.79 |
| 490 | CG | LEU | A | 59 | −6.681 | 20.100 | 8.634 | 1.00 | 25.86 |
| 491 | CD1 | LEU | A | 59 | −6.255 | 20.536 | 10.043 | 1.00 | 28.61 |
| 492 | CD2 | LEU | A | 59 | −8.165 | 19.799 | 8.572 | 1.00 | 29.27 |
| 493 | C | LEU | A | 59 | −6.014 | 21.943 | 5.278 | 1.00 | 16.23 |
| 494 | O | LEU | A | 59 | −5.156 | 22.737 | 5.653 | 1.00 | 15.43 |
| 495 | N | ALA | A | 60 | −6.583 | 21.999 | 4.086 | 1.00 | 13.19 |
| 496 | CA | ALA | A | 60 | −6.239 | 23.024 | 3.120 | 1.00 | 13.34 |
| 497 | CB | ALA | A | 60 | −6.709 | 22.585 | 1.734 | 1.00 | 13.93 |
| 498 | C | ALA | A | 60 | −6.921 | 24.318 | 3.490 | 1.00 | 14.71 |
| 499 | O | ALA | A | 60 | −8.139 | 24.348 | 3.708 | 1.00 | 13.73 |
| 500 | N | VAL | A | 61 | −6.149 | 25.401 | 3.543 | 1.00 | 12.02 |
| 501 | CA | VAL | A | 61 | −6.706 | 26.690 | 3.868 | 1.00 | 11.97 |
| 502 | CB | VAL | A | 61 | −6.482 | 27.058 | 5.390 | 1.00 | 11.44 |
| 503 | CG1 | VAL | A | 61 | −7.091 | 26.025 | 6.307 | 1.00 | 13.57 |
| 504 | CG2 | VAL | A | 61 | −4.945 | 27.222 | 5.686 | 1.00 | 13.14 |
| 505 | C | VAL | A | 61 | −6.094 | 27.827 | 3.074 | 1.00 | 11.99 |
| 506 | O | VAL | A | 61 | −5.007 | 27.698 | 2.474 | 1.00 | 10.58 |
| 507 | N | THR | A | 62 | −6.829 | 28.944 | 3.054 | 1.00 | 11.02 |
| 508 | CA | THR | A | 62 | −6.320 | 30.185 | 2.499 | 1.00 | 10.68 |
| 509 | CB | THR | A | 62 | −7.176 | 30.812 | 1.375 | 1.00 | 11.49 |
| 510 | OG1 | THR | A | 62 | −8.524 | 30.963 | 1.817 | 1.00 | 13.86 |
| 511 | CG2 | THR | A | 62 | −7.110 | 29.954 | 0.099 | 1.00 | 13.14 |
| 512 | C | THR | A | 62 | −6.369 | 31.133 | 3.688 | 1.00 | 11.80 |
| 513 | O | THR | A | 62 | −7.126 | 30.916 | 4.635 | 1.00 | 13.19 |
| 514 | N | LEU | A | 63 | −5.550 | 32.166 | 3.629 | 1.00 | 11.94 |
| 515 | CA | LEU | A | 63 | −5.485 | 33.166 | 4.687 | 1.00 | 11.08 |
| 516 | CB | LEU | A | 63 | −4.094 | 33.159 | 5.355 | 1.00 | 13.09 |
| 517 | CG | LEU | A | 63 | −3.809 | 32.100 | 6.426 | 1.00 | 17.87 |
| 518 | CD1 | LEU | A | 63 | −3.856 | 30.734 | 5.857 | 1.00 | 20.69 |
| 519 | CD2 | LEU | A | 63 | −2.453 | 32.365 | 7.057 | 1.00 | 17.94 |
| 520 | C | LEU | A | 63 | −5.718 | 34.521 | 4.041 | 1.00 | 11.67 |
| 521 | O | LEU | A | 63 | −5.040 | 34.866 | 3.078 | 1.00 | 11.40 |
| 522 | N | SER | A | 64 | −6.686 | 35.282 | 4.553 | 1.00 | 11.74 |
| 523 | CA | SER | A | 64 | −6.971 | 36.604 | 4.008 | 1.00 | 10.59 |
| 524 | CB | SER | A | 64 | −8.279 | 36.589 | 3.214 | 1.00 | 13.51 |
| 525 | OG | SER | A | 64 | −8.268 | 35.614 | 2.202 | 1.00 | 13.18 |
| 526 | C | SER | A | 64 | −7.168 | 37.639 | 5.109 | 1.00 | 12.92 |
| 527 | O | SER | A | 64 | −7.307 | 37.289 | 6.256 | 1.00 | 12.84 |
| 528 | N | VAL | A | 65 | −7.150 | 38.908 | 4.724 | 1.00 | 14.38 |
| 529 | CA | VAL | A | 65 | −7.456 | 39.992 | 5.662 | 1.00 | 15.02 |
| 530 | CB | VAL | A | 65 | −6.258 | 40.886 | 6.016 | 1.00 | 17.78 |
| 531 | CG1 | VAL | A | 65 | −5.343 | 40.128 | 6.952 | 1.00 | 17.73 |
| 532 | CG2 | VAL | A | 65 | −5.544 | 41.350 | 4.780 | 1.00 | 18.38 |
| 533 | C | VAL | A | 65 | −8.490 | 40.814 | 4.918 | 1.00 | 16.31 |
| 534 | O | VAL | A | 65 | −8.463 | 40.911 | 3.692 | 1.00 | 17.00 |
| 535 | N | LYS | A | 66 | −9.408 | 41.401 | 5.662 | 1.00 | 17.79 |
| 536 | CA | LYS | A | 66 | −10.451 | 42.184 | 5.051 | 1.00 | 18.90 |
| 537 | CB | LYS | A | 66 | −11.802 | 41.576 | 5.435 | 1.00 | 20.44 |
| 538 | CG | LYS | A | 66 | −13.039 | 42.410 | 5.113 | 1.00 | 23.51 |
| 539 | CD | LYS | A | 66 | −13.148 | 42.708 | 3.642 | 1.00 | 28.98 |
| 540 | CE | LYS | A | 66 | −14.414 | 43.512 | 3.375 | 1.00 | 30.87 |
| 541 | NZ | LYS | A | 66 | −15.582 | 42.846 | 4.020 | 1.00 | 35.82 |
| 542 | C | LYS | A | 66 | −10.341 | 43.628 | 5.506 | 1.00 | 21.57 |
| 543 | O | LYS | A | 66 | −10.506 | 43.928 | 6.681 | 1.00 | 21.71 |
| 544 | N | ASP | A | 67 | −9.996 | 44.504 | 4.574 | 1.00 | 24.49 |
| 545 | CA | ASP | A | 67 | −9.925 | 45.933 | 4.855 | 1.00 | 28.29 |
| 546 | CB | ASP | A | 67 | −8.745 | 46.569 | 4.113 | 1.00 | 33.32 |
| 547 | CG | ASP | A | 67 | −8.227 | 47.848 | 4.797 | 1.00 | 38.19 |
| 548 | OD1 | ASP | A | 67 | −7.180 | 48.360 | 4.327 | 1.00 | 40.49 |
| 549 | OD2 | ASP | A | 67 | −8.846 | 48.341 | 5.789 | 1.00 | 38.08 |
| 550 | C | ASP | A | 67 | −11.256 | 46.338 | 4.229 | 1.00 | 30.38 |
| 551 | O | ASP | A | 67 | −12.322 | 45.957 | 4.730 | 1.00 | 33.54 |
| 552 | N | SER | A | 68 | −11.223 | 47.075 | 3.131 | 1.00 | 29.94 |
| 553 | CA | SER | A | 68 | −12.482 | 47.428 | 2.478 | 1.00 | 29.05 |
| 554 | CB | SER | A | 68 | −12.290 | 48.614 | 1.533 | 1.00 | 30.55 |
| 555 | OG | SER | A | 68 | −13.521 | 48.968 | 0.924 | 1.00 | 31.57 |
| 556 | C | SER | A | 68 | −12.837 | 46.175 | 1.689 | 1.00 | 28.80 |
| 557 | O | SER | A | 68 | −13.994 | 45.766 | 1.586 | 1.00 | 28.69 |
| 558 | N | LYS | A | 69 | −11.815 | 45.571 | 1.111 | 1.00 | 27.30 |
| 559 | CA | LYS | A | 69 | −12.024 | 44.354 | 0.374 | 1.00 | 27.76 |
| 560 | CB | LYS | A | 69 | −11.838 | 44.582 | −1.129 | 1.00 | 29.95 |
| 561 | CG | LYS | A | 69 | −10.524 | 45.197 | −1.551 | 1.00 | 31.83 |
| 562 | CD | LYS | A | 69 | −10.740 | 46.148 | −2.748 | 1.00 | 34.15 |
| 563 | CE | LYS | A | 69 | −11.716 | 45.593 | −3.800 | 1.00 | 35.45 |
| 564 | NZ | LYS | A | 69 | −11.122 | 44.605 | −4.752 | 1.00 | 37.07 |
| 565 | C | LYS | A | 69 | −11.018 | 43.366 | 0.923 | 1.00 | 25.25 |
| 566 | O | LYS | A | 69 | −10.234 | 43.694 | 1.812 | 1.00 | 26.87 |
| 567 | N | MET | A | 70 | −11.068 | 42.151 | 0.412 | 1.00 | 23.69 |
| 568 | CA | MET | A | 70 | −10.175 | 41.116 | 0.881 | 1.00 | 21.35 |
| 569 | CB | MET | A | 70 | −10.850 | 39.751 | 0.703 | 1.00 | 23.05 |
| 570 | CG | AMET | A | 70 | −12.146 | 39.571 | 1.446 | 0.50 | 23.06 |
| 571 | CG | BMET | A | 70 | −12.095 | 39.633 | 1.548 | 0.50 | 24.64 |
| 572 | SD | AMET | A | 70 | −11.838 | 38.805 | 3.017 | 0.50 | 24.93 |
| 573 | SD | BMET | A | 70 | −12.835 | 38.037 | 1.426 | 0.50 | 27.70 |
| 574 | CE | AMET | A | 70 | −12.150 | 37.121 | 2.609 | 0.50 | 21.42 |
| 575 | CE | BMET | A | 70 | −11.893 | 37.163 | 2.620 | 0.50 | 23.38 |
| 576 | C | MET | A | 70 | −8.859 | 41.123 | 0.133 | 1.00 | 19.52 |
| 577 | O | MET | A | 70 | −8.781 | 41.541 | −1.020 | 1.00 | 19.18 |
| 578 | N | SER | A | 71 | −7.817 | 40.677 | 0.825 | 1.00 | 15.82 |
| 579 | CA | SER | A | 71 | −6.494 | 40.496 | 0.238 | 1.00 | 16.42 |
| 580 | CB | ASER | A | 71 | −5.477 | 41.489 | 0.799 | 0.50 | 18.42 |
| 581 | CB | BSER | A | 71 | −5.459 | 41.515 | 0.725 | 0.50 | 18.71 |
| 582 | OG | ASER | A | 71 | −5.721 | 42.787 | 0.281 | 0.50 | 16.45 |
| 583 | OG | BSER | A | 71 | −5.184 | 41.396 | 2.094 | 0.50 | 18.52 |
| 584 | C | SER | A | 71 | −6.212 | 39.068 | 0.724 | 1.00 | 16.36 |
| 585 | O | SER | A | 71 | −6.598 | 38.691 | 1.839 | 1.00 | 16.21 |
| 586 | N | THR | A | 72 | −5.599 | 38.260 | −0.124 | 1.00 | 13.02 |
| 587 | CA | THR | A | 72 | −5.342 | 36.877 | 0.234 | 1.00 | 12.17 |
| 588 | CB | THR | A | 72 | −6.174 | 35.967 | −0.681 | 1.00 | 14.46 |
| 589 | OG1 | THR | A | 72 | −7.567 | 36.233 | −0.447 | 1.00 | 14.61 |
| 590 | CG2 | THR | A | 72 | −5.895 | 34.510 | −0.426 | 1.00 | 15.94 |
| 591 | C | THR | A | 72 | −3.853 | 36.580 | 0.124 | 1.00 | 12.63 |
| 592 | O | THR | A | 72 | −3.179 | 37.045 | −0.786 | 1.00 | 13.57 |
| 593 | N | LEU | A | 73 | −3.353 | 35.812 | 1.084 | 1.00 | 11.63 |
| 594 | CA | LEU | A | 73 | −1.956 | 35.450 | 1.120 | 1.00 | 11.94 |
| 595 | CB | LEU | A | 73 | −1.670 | 34.720 | 2.433 | 1.00 | 12.27 |
| 596 | CG | LEU | A | 73 | −0.246 | 34.154 | 2.583 | 1.00 | 11.98 |
| 597 | CD1 | LEU | A | 73 | 0.785 | 35.251 | 2.517 | 1.00 | 15.22 |
| 598 | CD2 | LEU | A | 73 | −0.158 | 33.380 | 3.924 | 1.00 | 14.43 |
| 599 | C | LEU | A | 73 | −1.584 | 34.537 | −0.053 | 1.00 | 12.95 |
| 600 | O | LEU | A | 73 | −2.272 | 33.558 | −0.333 | 1.00 | 12.53 |
| 601 | N | SER | A | 74 | −0.498 | 34.899 | −0.726 | 1.00 | 12.08 |
| 602 | CA | SER | A | 74 | 0.063 | 34.120 | −1.825 | 1.00 | 13.43 |
| 603 | CB | SER | A | 74 | −0.071 | 34.870 | −3.147 | 1.00 | 15.37 |
| 604 | OG | SER | A | 74 | 0.785 | 34.281 | −4.139 | 1.00 | 19.98 |
| 605 | C | SER | A | 74 | 1.548 | 33.926 | −1.541 | 1.00 | 11.44 |
| 606 | O | SER | A | 74 | 2.184 | 34.790 | −0.954 | 1.00 | 13.15 |
| 607 | N | CYS | A | 75 | 2.095 | 32.788 | −1.959 | 1.00 | 12.82 |
| 608 | CA | CYS | A | 75 | 3.527 | 32.543 | −1.805 | 1.00 | 12.41 |
| 609 | CB | CYS | A | 75 | 3.803 | 31.345 | −0.889 | 1.00 | 15.03 |
| 610 | SG | CYS | A | 75 | 3.246 | 29.737 | −1.493 | 1.00 | 14.95 |
| 611 | C | CYS | A | 75 | 4.123 | 32.288 | −3.198 | 1.00 | 15.39 |
| 612 | O | CYS | A | 75 | 5.152 | 31.626 | −3.340 | 1.00 | 15.73 |
| 613 | N | LYS | A | 76 | 3.471 | 32.846 | −4.213 | 1.00 | 17.25 |
| 614 | CA | LYS | A | 76 | 3.931 | 32.685 | −5.592 | 1.00 | 17.87 |
| 615 | CB | LYS | A | 76 | 3.117 | 33.580 | −6.516 | 1.00 | 19.69 |
| 616 | CG | LYS | A | 76 | 3.336 | 33.258 | −7.994 | 1.00 | 24.67 |
| 617 | CD | LYS | A | 76 | 2.275 | 33.908 | −8.863 | 1.00 | 27.85 |
| 618 | CE | LYS | A | 76 | 2.408 | 35.430 | −8.875 | 1.00 | 31.65 |
| 619 | NZ | LYS | A | 76 | 1.558 | 36.045 | −9.931 | 1.00 | 34.74 |
| 620 | C | LYS | A | 76 | 5.424 | 33.018 | −5.715 | 1.00 | 17.82 |
| 621 | O | LYS | A | 76 | 5.892 | 34.025 | −5.190 | 1.00 | 18.79 |
| 622 | N | ASN | A | 77 | 6.160 | 32.143 | −6.399 | 1.00 | 20.58 |
| 623 | CA | ASN | A | 77 | 7.600 | 32.310 | −6.607 | 1.00 | 21.23 |
| 624 | CB | ASN | A | 77 | 7.864 | 33.596 | −7.405 | 1.00 | 23.75 |
| 625 | CG | ASN | A | 77 | 7.086 | 33.645 | −8.710 | 1.00 | 27.51 |
| 626 | OD1 | ASN | A | 77 | 6.388 | 34.630 | −8.992 | 1.00 | 32.76 |
| 627 | ND2 | ASN | A | 77 | 7.197 | 32.584 | −9.516 | 1.00 | 27.56 |
| 628 | C | ASN | A | 77 | 8.370 | 32.359 | −5.288 | 1.00 | 20.98 |
| 629 | O | ASN | A | 77 | 9.431 | 32.979 | −5.197 | 1.00 | 21.46 |
| 630 | N | LYS | A | 78 | 7.812 | 31.697 | −4.270 | 1.00 | 19.95 |
| 631 | CA | LYS | A | 78 | 8.385 | 31.628 | −2.928 | 1.00 | 18.97 |
| 632 | CB | LYS | A | 78 | 9.746 | 30.922 | −2.955 | 1.00 | 21.56 |
| 633 | CG | LYS | A | 78 | 9.657 | 29.544 | −3.583 | 1.00 | 22.77 |
| 634 | CD | LYS | A | 78 | 10.903 | 28.709 | −3.308 | 1.00 | 23.94 |
| 635 | CE | LYS | A | 78 | 10.804 | 27.355 | −3.959 | 1.00 | 26.35 |
| 636 | NZ | LYS | A | 78 | 11.078 | 27.439 | −5.425 | 1.00 | 33.16 |
| 637 | C | LYS | A | 78 | 8.501 | 32.977 | −2.264 | 1.00 | 18.94 |
| 638 | O | LYS | A | 78 | 9.322 | 33.175 | −1.360 | 1.00 | 19.31 |
| 639 | N | ILE | A | 79 | 7.675 | 33.913 | −2.721 | 1.00 | 16.05 |
| 640 | CA | ILE | A | 79 | 7.657 | 35.255 | −2.150 | 1.00 | 16.85 |
| 641 | CB | ILE | A | 79 | 7.897 | 36.343 | −3.228 | 1.00 | 19.97 |
| 642 | CG2 | ILE | A | 79 | 7.801 | 37.725 | −2.600 | 1.00 | 21.54 |
| 643 | CG1 | ILE | A | 79 | 9.267 | 36.121 | −3.871 | 1.00 | 22.28 |
| 644 | CD1 | ILE | A | 79 | 10.432 | 36.146 | −2.884 | 1.00 | 24.28 |
| 645 | C | ILE | A | 79 | 6.283 | 35.472 | −1.515 | 1.00 | 14.59 |
| 646 | O | ILE | A | 79 | 5.266 | 35.197 | −2.123 | 1.00 | 16.80 |
| 647 | N | ILE | A | 80 | 6.267 | 35.981 | −0.290 | 1.00 | 15.26 |
| 648 | CA | ILE | A | 80 | 5.008 | 36.217 | 0.396 | 1.00 | 15.28 |
| 649 | CB | ILE | A | 80 | 5.213 | 36.214 | 1.916 | 1.00 | 15.42 |
| 650 | CG2 | ILE | A | 80 | 3.950 | 36.775 | 2.617 | 1.00 | 12.93 |
| 651 | CG1 | ILE | A | 80 | 5.629 | 34.819 | 2.387 | 1.00 | 15.28 |
| 652 | CD1 | ILE | A | 80 | 4.619 | 33.723 | 2.130 | 1.00 | 13.88 |
| 653 | C | ILE | A | 80 | 4.391 | 37.562 | 0.021 | 1.00 | 16.46 |
| 654 | O | ILE | A | 80 | 5.064 | 38.578 | 0.004 | 1.00 | 18.29 |
| 655 | N | SER | A | 81 | 3.099 | 37.553 | −0.278 | 1.00 | 15.80 |
| 656 | CA | SER | A | 81 | 2.382 | 38.787 | −0.573 | 1.00 | 16.94 |
| 657 | CB | SER | A | 81 | 2.521 | 39.172 | −2.049 | 1.00 | 19.27 |
| 658 | OG | SER | A | 81 | 1.613 | 38.442 | −2.839 | 1.00 | 23.09 |
| 659 | C | SER | A | 81 | 0.912 | 38.565 | −0.283 | 1.00 | 17.78 |
| 660 | O | SER | A | 81 | 0.429 | 37.436 | −0.261 | 1.00 | 18.89 |
| 661 | N | PHE | A | 82 | 0.207 | 39.651 | −0.024 | 1.00 | 15.45 |
| 662 | CA | PHE | A | 82 | −1.226 | 39.564 | 0.151 | 1.00 | 14.77 |
| 663 | CB | PHE | A | 82 | −1.703 | 40.282 | 1.413 | 1.00 | 15.41 |
| 664 | CG | PHE | A | 82 | −1.671 | 39.404 | 2.626 | 1.00 | 12.72 |
| 665 | CD1 | PHE | A | 82 | −0.467 | 39.124 | 3.263 | 1.00 | 13.82 |
| 666 | CD2 | PHE | A | 82 | −2.839 | 38.832 | 3.113 | 1.00 | 14.26 |
| 667 | CE1 | PHE | A | 82 | −0.439 | 38.288 | 4.376 | 1.00 | 14.72 |
| 668 | CE2 | PHE | A | 82 | −2.811 | 37.992 | 4.230 | 1.00 | 13.40 |
| 669 | CZ | PHE | A | 82 | −1.603 | 37.728 | 4.855 | 1.00 | 13.52 |
| 670 | C | PHE | A | 82 | −1.706 | 40.253 | −1.104 | 1.00 | 17.25 |
| 671 | O | PHE | A | 82 | −1.470 | 41.450 | −1.298 | 1.00 | 18.41 |
| 672 | N | GLU | A | 83 | −2.327 | 39.462 | −1.971 | 1.00 | 19.38 |
| 673 | CA | GLU | A | 83 | −2.837 | 39.940 | −3.248 | 1.00 | 19.61 |
| 674 | CB | GLU | A | 83 | −2.856 | 38.808 | −4.274 | 1.00 | 21.27 |
| 675 | CG | AGLU | A | 83 | −1.507 | 38.161 | −4.521 | 0.50 | 24.59 |
| 676 | CG | BGLU | A | 83 | −1.487 | 38.192 | −4.581 | 0.50 | 24.80 |
| 677 | CD | AGLU | A | 83 | −1.571 | 37.088 | −5.587 | 0.50 | 25.84 |
| 678 | CD | BGLU | A | 83 | −0.565 | 39.119 | −5.358 | 0.50 | 26.64 |
| 679 | OE1 | AGLU | A | 83 | −2.478 | 36.237 | −5.517 | 0.50 | 26.32 |
| 680 | OE1 | BGLU | A | 83 | −0.959 | 39.570 | −6.455 | 0.50 | 28.76 |
| 681 | OE2 | AGLU | A | 83 | −0.711 | 37.094 | −6.493 | 0.50 | 29.51 |
| 682 | OE2 | BGLU | A | 83 | 0.558 | 39.391 | −4.882 | 0.50 | 28.12 |
| 683 | C | GLU | A | 83 | −4.246 | 40.455 | −3.087 | 1.00 | 19.90 |
| 684 | O | GLU | A | 83 | −5.044 | 39.892 | −2.358 | 1.00 | 17.94 |
| 685 | N | GLU | A | 84 | −4.557 | 41.525 | −3.808 | 1.00 | 22.59 |
| 686 | CA | GLU | A | 84 | −5.884 | 42.101 | −3.742 | 1.00 | 24.32 |
| 687 | CB | GLU | A | 84 | −5.868 | 43.477 | −4.415 | 1.00 | 25.42 |
| 688 | CG | AGLU | A | 84 | −4.854 | 44.427 | −3.817 | 0.50 | 27.52 |
| 689 | CG | BGLU | A | 84 | −6.481 | 44.542 | −3.554 | 0.50 | 28.22 |
| 690 | CD | AGLU | A | 84 | −4.657 | 45.666 | −4.663 | 0.50 | 29.00 |
| 691 | CD | BGLU | A | 84 | −7.722 | 44.044 | −2.866 | 0.50 | 28.45 |
| 692 | OE1 | AGLU | A | 84 | −3.624 | 45.751 | −5.360 | 0.50 | 28.85 |
| 693 | OE1 | BGLU | A | 84 | −8.694 | 43.722 | −3.573 | 0.50 | 31.66 |
| 694 | OE2 | AGLU | A | 84 | −5.541 | 46.545 | −4.635 | 0.50 | 29.98 |
| 695 | OE2 | BGLU | A | 84 | −7.717 | 43.961 | −1.624 | 0.50 | 29.55 |
| 696 | C | GLU | A | 84 | −6.865 | 41.176 | −4.461 | 1.00 | 24.42 |
| 697 | O | GLU | A | 84 | −7.124 | 41.341 | −5.658 | 1.00 | 27.68 |
| 698 | N | MET | A | 85 | −7.418 | 40.214 | −3.735 | 1.00 | 22.01 |
| 699 | CA | MET | A | 85 | −8.362 | 39.274 | −4.322 | 1.00 | 22.67 |
| 700 | CB | MET | A | 85 | −7.618 | 38.303 | −5.238 | 1.00 | 24.97 |
| 701 | CG | MET | A | 85 | −6.720 | 37.352 | −4.459 | 1.00 | 26.77 |
| 702 | SD | MET | A | 85 | −5.752 | 36.259 | −5.486 | 1.00 | 33.90 |
| 703 | CE | MET | A | 85 | −6.974 | 35.128 | −6.021 | 1.00 | 30.61 |
| 704 | C | MET | A | 85 | −9.057 | 38.473 | −3.236 | 1.00 | 21.35 |
| 705 | O | MET | A | 85 | −8.514 | 38.300 | −2.141 | 1.00 | 19.24 |
| 706 | N | ASP | A | 86 | −10.257 | 37.989 | −3.522 | 1.00 | 18.17 |
| 707 | CA | ASP | A | 86 | −10.980 | 37.175 | −2.553 | 1.00 | 20.10 |
| 708 | CB | ASP | A | 86 | −12.477 | 37.123 | −2.854 | 1.00 | 21.34 |
| 709 | CG | ASP | A | 86 | −13.167 | 38.446 | −2.698 | 1.00 | 22.68 |
| 710 | OD1 | ASP | A | 86 | −12.578 | 39.395 | −2.158 | 1.00 | 22.70 |
| 711 | OD2 | ASP | A | 86 | −14.336 | 38.514 | −3.125 | 1.00 | 27.94 |
| 712 | C | ASP | A | 86 | −10.472 | 35.745 | −2.692 | 1.00 | 18.80 |
| 713 | O | ASP | A | 86 | −9.958 | 35.372 | −3.735 | 1.00 | 20.14 |
| 714 | N | PRO | A | 87 | −10.610 | 34.930 | −1.639 | 1.00 | 18.18 |
| 715 | CD | PRO | A | 87 | −10.995 | 35.279 | −0.263 | 1.00 | 16.11 |
| 716 | CA | PRO | A | 87 | −10.155 | 33.533 | −1.732 | 1.00 | 17.40 |
| 717 | CB | PRO | A | 87 | −10.450 | 32.972 | −0.345 | 1.00 | 18.66 |
| 718 | CG | PRO | A | 87 | −10.432 | 34.147 | 0.526 | 1.00 | 20.76 |
| 719 | C | PRO | A | 87 | −11.096 | 32.915 | −2.767 | 1.00 | 18.53 |
| 720 | O | PRO | A | 87 | −12.299 | 33.016 | −2.626 | 1.00 | 20.16 |
| 721 | N | PRO | A | 88 | −10.558 | 32.250 | −3.795 | 1.00 | 18.80 |
| 722 | CD | PRO | A | 88 | −9.136 | 31.980 | −4.073 | 1.00 | 18.15 |
| 723 | CA | PRO | A | 88 | −11.424 | 31.651 | −4.819 | 1.00 | 17.32 |
| 724 | CB | PRO | A | 88 | −10.416 | 31.134 | −5.854 | 1.00 | 18.71 |
| 725 | CG | PRO | A | 88 | −9.211 | 30.806 | −5.034 | 1.00 | 21.64 |
| 726 | C | PRO | A | 88 | −12.415 | 30.576 | −4.363 | 1.00 | 18.89 |
| 727 | O | PRO | A | 88 | −12.139 | 29.787 | −3.467 | 1.00 | 18.86 |
| 728 | N | GLU | A | 89 | −13.590 | 30.567 | −4.989 | 1.00 | 18.05 |
| 729 | CA | GLU | A | 89 | −14.609 | 29.574 | −4.682 | 1.00 | 18.83 |
| 730 | CB | GLU | A | 89 | −15.853 | 29.812 | −5.531 | 1.00 | 22.49 |
| 731 | CG | GLU | A | 89 | −16.405 | 31.187 | −5.413 | 1.00 | 29.78 |
| 732 | CD | GLU | A | 89 | −16.970 | 31.418 | −4.049 | 1.00 | 35.11 |
| 733 | OE1 | GLU | A | 89 | −17.382 | 30.413 | −3.431 | 1.00 | 35.91 |
| 734 | OE2 | GLU | A | 89 | −17.013 | 32.595 | −3.605 | 1.00 | 36.99 |
| 735 | C | GLU | A | 89 | −14.059 | 28.208 | −5.056 | 1.00 | 15.39 |
| 736 | O | GLU | A | 89 | −14.313 | 27.216 | −4.392 | 1.00 | 17.46 |
| 737 | N | ASN | A | 90 | −13.324 | 28.186 | −6.158 | 1.00 | 16.43 |
| 738 | CA | ASN | A | 90 | −12.747 | 26.967 | −6.682 | 1.00 | 17.28 |
| 739 | CB | ASN | A | 90 | −13.619 | 26.442 | −7.815 | 1.00 | 15.51 |
| 740 | CG | ASN | A | 90 | −14.992 | 26.112 | −7.362 | 1.00 | 14.75 |
| 741 | OD1 | ASN | A | 90 | −15.188 | 25.128 | −6.678 | 1.00 | 17.59 |
| 742 | ND2 | ASN | A | 90 | −15.973 | 26.954 | −7.725 | 1.00 | 16.75 |
| 743 | C | ASN | A | 90 | −11.364 | 27.197 | −7.235 | 1.00 | 16.58 |
| 744 | O | ASN | A | 90 | −11.081 | 28.238 | −7.820 | 1.00 | 18.55 |
| 745 | N | ILE | A | 91 | −10.492 | 26.211 | −7.035 | 1.00 | 18.81 |
| 746 | CA | ILE | A | 91 | −9.134 | 26.252 | −7.557 | 1.00 | 18.68 |
| 747 | CB | ILE | A | 91 | −8.080 | 26.301 | −6.443 | 1.00 | 19.32 |
| 748 | CG2 | ILE | A | 91 | −6.708 | 26.363 | −7.058 | 1.00 | 17.89 |
| 749 | CG1 | ILE | A | 91 | −8.320 | 27.511 | −5.546 | 1.00 | 17.34 |
| 750 | CD1 | ILE | A | 91 | −7.370 | 27.577 | −4.335 | 1.00 | 17.37 |
| 751 | C | ILE | A | 91 | −9.047 | 24.939 | −8.301 | 1.00 | 21.82 |
| 752 | O | ILE | A | 91 | −9.069 | 23.867 | −7.699 | 1.00 | 20.95 |
| 753 | N | ASP | A | 92 | −8.959 | 25.032 | −9.614 | 1.00 | 20.54 |
| 754 | CA | ASP | A | 92 | −8.972 | 23.837 | −10.433 | 1.00 | 25.57 |
| 755 | CB | ASP | A | 92 | −9.876 | 24.113 | −11.646 | 1.00 | 24.59 |
| 756 | CG | ASP | A | 92 | −11.160 | 24.911 | −11.271 | 1.00 | 24.09 |
| 757 | OD1 | ASP | A | 92 | −12.057 | 24.413 | −10.612 | 1.00 | 15.45 |
| 758 | OD2 | ASP | A | 92 | −11.272 | 26.088 | −11.648 | 1.00 | 30.89 |
| 759 | C | ASP | A | 92 | −7.566 | 23.354 | −10.801 | 1.00 | 24.67 |
| 760 | O | ASP | A | 92 | −7.163 | 23.286 | −11.968 | 1.00 | 28.38 |
| 761 | N | ASP | A | 93 | −6.809 | 23.028 | −9.760 | 1.00 | 22.69 |
| 762 | CA | ASP | A | 93 | −5.449 | 22.529 | −9.903 | 1.00 | 23.13 |
| 763 | CB | ASP | A | 93 | −4.449 | 23.678 | −10.104 | 1.00 | 26.05 |
| 764 | CG | ASP | A | 93 | −3.147 | 23.215 | −10.734 | 1.00 | 29.72 |
| 765 | OD1 | ASP | A | 93 | −2.382 | 22.463 | −10.090 | 1.00 | 29.89 |
| 766 | OD2 | ASP | A | 93 | −2.892 | 23.592 | −11.903 | 1.00 | 34.67 |
| 767 | C | ASP | A | 93 | −5.140 | 21.803 | −8.604 | 1.00 | 21.08 |
| 768 | O | ASP | A | 93 | −5.848 | 21.983 | −7.617 | 1.00 | 20.24 |
| 769 | N | ILE | A | 94 | −4.097 | 20.977 | −8.591 | 1.00 | 20.15 |
| 770 | CA | ILE | A | 94 | −3.769 | 20.261 | −7.367 | 1.00 | 20.98 |
| 771 | CB | ILE | A | 94 | −3.184 | 18.856 | −7.635 | 1.00 | 23.61 |
| 772 | CG2 | ILE | A | 94 | −4.311 | 17.914 | −8.043 | 1.00 | 23.40 |
| 773 | CG1 | ILE | A | 94 | −2.089 | 18.920 | −8.701 | 1.00 | 24.17 |
| 774 | CD1 | ILE | A | 94 | −1.342 | 17.583 | −8.869 | 1.00 | 28.22 |
| 775 | C | ILE | A | 94 | −2.792 | 21.047 | −6.510 | 1.00 | 21.87 |
| 776 | O | ILE | A | 94 | −2.474 | 20.658 | −5.396 | 1.00 | 21.96 |
| 777 | N | GLN | A | 95 | −2.318 | 22.162 | −7.037 | 1.00 | 19.02 |
| 778 | CA | GLN | A | 95 | −1.398 | 23.004 | −6.299 | 1.00 | 19.98 |
| 779 | CB | GLN | A | 95 | 0.034 | 22.847 | −6.818 | 1.00 | 23.97 |
| 780 | CG | GLN | A | 95 | 0.527 | 21.415 | −6.759 | 1.00 | 28.41 |
| 781 | CD | GLN | A | 95 | 1.969 | 21.269 | −7.173 | 1.00 | 32.45 |
| 782 | OE1 | GLN | A | 95 | 2.346 | 21.637 | −8.286 | 1.00 | 34.02 |
| 783 | NE2 | GLN | A | 95 | 2.794 | 20.730 | −6.272 | 1.00 | 34.91 |
| 784 | C | GLN | A | 95 | −1.829 | 24.432 | −6.468 | 1.00 | 17.22 |
| 785 | O | GLN | A | 95 | −2.364 | 24.814 | −7.503 | 1.00 | 19.35 |
| 786 | N | SER | A | 96 | −1.585 | 25.237 | −5.451 | 1.00 | 15.97 |
| 787 | CA | SER | A | 96 | −1.931 | 26.638 | −5.530 | 1.00 | 16.16 |
| 788 | CB | SER | A | 96 | −3.374 | 26.856 | −5.109 | 1.00 | 15.85 |
| 789 | OG | SER | A | 96 | −3.654 | 28.241 | −4.954 | 1.00 | 14.88 |
| 790 | C | SER | A | 96 | −1.037 | 27.439 | −4.619 | 1.00 | 14.50 |
| 791 | O | SER | A | 96 | −0.720 | 27.001 | −3.513 | 1.00 | 16.46 |
| 792 | N | ASP | A | 97 | −0.654 | 28.618 | −5.081 | 1.00 | 15.93 |
| 793 | CA | ASP | A | 97 | 0.161 | 29.517 | −4.294 | 1.00 | 15.84 |
| 794 | CB | ASP | A | 97 | 0.701 | 30.645 | −5.169 | 1.00 | 17.89 |
| 795 | CG | ASP | A | 97 | −0.405 | 31.445 | −5.841 | 1.00 | 15.35 |
| 796 | OD1 | ASP | A | 97 | −1.282 | 30.833 | −6.460 | 1.00 | 23.15 |
| 797 | OD2 | ASP | A | 97 | −0.400 | 32.690 | −5.766 | 1.00 | 22.26 |
| 798 | C | ASP | A | 97 | −0.662 | 30.128 | −3.154 | 1.00 | 13.09 |
| 799 | O | ASP | A | 97 | −0.104 | 30.825 | −2.319 | 1.00 | 12.86 |
| 800 | N | LEU | A | 98 | −1.971 | 29.864 | −3.135 | 1.00 | 13.02 |
| 801 | CA | LEU | A | 98 | −2.877 | 30.407 | −2.110 | 1.00 | 12.02 |
| 802 | CB | LEU | A | 98 | −4.195 | 30.859 | −2.758 | 1.00 | 13.29 |
| 803 | CG | LEU | A | 98 | −4.103 | 31.759 | −3.983 | 1.00 | 13.93 |
| 804 | CD1 | LEU | A | 98 | −5.525 | 32.047 | −4.475 | 1.00 | 17.40 |
| 805 | CD2 | LEU | A | 98 | −3.359 | 33.034 | −3.657 | 1.00 | 14.69 |
| 806 | C | LEU | A | 98 | −3.213 | 29.381 | −1.049 | 1.00 | 11.66 |
| 807 | O | LEU | A | 98 | −3.682 | 29.721 | 0.035 | 1.00 | 11.48 |
| 808 | N | ILE | A | 99 | −3.015 | 28.112 | −1.368 | 1.00 | 11.13 |
| 809 | CA | ILE | A | 99 | −3.363 | 27.070 | −0.428 | 1.00 | 11.30 |
| 810 | CB | ILE | A | 99 | −3.955 | 25.869 | −1.160 | 1.00 | 11.91 |
| 811 | CG2 | ILE | A | 99 | −4.089 | 24.703 | −0.208 | 1.00 | 13.29 |
| 812 | CG1 | ILE | A | 99 | −5.301 | 26.260 | −1.800 | 1.00 | 13.89 |
| 813 | CD1 | ILE | A | 99 | −6.453 | 26.451 | −0.813 | 1.00 | 14.88 |
| 814 | C | ILE | A | 99 | −2.230 | 26.559 | 0.443 | 1.00 | 12.54 |
| 815 | O | ILE | A | 99 | −1.161 | 26.216 | −0.049 | 1.00 | 12.28 |
| 816 | N | PHE | A | 100 | −2.487 | 26.512 | 1.743 | 1.00 | 11.42 |
| 817 | CA | PHE | A | 100 | −1.515 | 25.978 | 2.684 | 1.00 | 12.45 |
| 818 | CB | PHE | A | 100 | −1.068 | 27.063 | 3.664 | 1.00 | 11.81 |
| 819 | CG | PHE | A | 100 | −0.371 | 28.186 | 2.999 | 1.00 | 13.04 |
| 820 | CD1 | PHE | A | 100 | −1.092 | 29.202 | 2.373 | 1.00 | 12.97 |
| 821 | CD2 | PHE | A | 100 | 1.016 | 28.189 | 2.897 | 1.00 | 11.78 |
| 822 | CE1 | PHE | A | 100 | −0.432 | 30.207 | 1.647 | 1.00 | 12.11 |
| 823 | CE2 | PHE | A | 100 | 1.668 | 29.191 | 2.172 | 1.00 | 14.15 |
| 824 | CZ | PHE | A | 100 | 0.954 | 30.195 | 1.547 | 1.00 | 13.64 |
| 825 | C | PHE | A | 100 | −2.195 | 24.862 | 3.434 | 1.00 | 13.20 |
| 826 | O | PHE | A | 100 | −3.410 | 24.879 | 3.617 | 1.00 | 13.09 |
| 827 | N | PHE | A | 101 | −1.429 | 23.880 | 3.871 | 1.00 | 10.67 |
| 828 | CA | PHE | A | 101 | −2.025 | 22.814 | 4.642 | 1.00 | 12.08 |
| 829 | CB | PHE | A | 101 | −1.497 | 21.469 | 4.157 | 1.00 | 13.01 |
| 830 | CG | PHE | A | 101 | −2.049 | 21.069 | 2.813 | 1.00 | 12.10 |
| 831 | CD1 | PHE | A | 101 | −3.361 | 20.617 | 2.706 | 1.00 | 14.27 |
| 832 | CD2 | PHE | A | 101 | −1.263 | 21.164 | 1.673 | 1.00 | 13.60 |
| 833 | CE1 | PHE | A | 101 | −3.894 | 20.258 | 1.453 | 1.00 | 13.74 |
| 834 | CE2 | PHE | A | 101 | −1.795 | 20.803 | 0.422 | 1.00 | 13.76 |
| 835 | CZ | PHE | A | 101 | −3.101 | 20.357 | 0.335 | 1.00 | 13.40 |
| 836 | C | PHE | A | 101 | −1.627 | 23.036 | 6.085 | 1.00 | 11.97 |
| 837 | O | PHE | A | 101 | −0.443 | 23.136 | 6.378 | 1.00 | 13.44 |
| 838 | N | GLN | A | 102 | −2.610 | 23.140 | 6.971 | 1.00 | 12.46 |
| 839 | CA | GLN | A | 102 | −2.347 | 23.322 | 8.403 | 1.00 | 14.12 |
| 840 | CB | GLN | A | 102 | −3.602 | 23.739 | 9.149 | 1.00 | 17.33 |
| 841 | CG | GLN | A | 102 | −4.239 | 24.988 | 8.661 | 1.00 | 16.36 |
| 842 | CD | GLN | A | 102 | −5.422 | 25.364 | 9.526 | 1.00 | 17.78 |
| 843 | OE1 | GLN | A | 102 | −6.255 | 24.521 | 9.850 | 1.00 | 22.15 |
| 844 | NE2 | GLN | A | 102 | −5.496 | 26.620 | 9.908 | 1.00 | 16.96 |
| 845 | C | GLN | A | 102 | −1.904 | 22.015 | 8.989 | 1.00 | 16.30 |
| 846 | O | GLN | A | 102 | −2.579 | 20.995 | 8.827 | 1.00 | 19.92 |
| 847 | N | LYS | A | 103 | −0.784 | 22.030 | 9.694 | 1.00 | 15.11 |
| 848 | CA | LYS | A | 103 | −0.243 | 20.804 | 10.293 | 1.00 | 14.68 |
| 849 | CB | LYS | A | 103 | 1.128 | 20.486 | 9.704 | 1.00 | 15.14 |
| 850 | CG | LYS | A | 103 | 1.151 | 20.364 | 8.209 | 1.00 | 16.52 |
| 851 | CD | LYS | A | 103 | 0.473 | 19.102 | 7.782 | 1.00 | 20.19 |
| 852 | CE | LYS | A | 103 | 0.539 | 18.973 | 6.275 | 1.00 | 18.98 |
| 853 | NZ | LYS | A | 103 | −0.055 | 17.656 | 5.865 | 1.00 | 25.28 |
| 854 | C | LYS | A | 103 | −0.063 | 21.012 | 11.768 | 1.00 | 16.97 |
| 855 | O | LYS | A | 103 | 0.692 | 21.896 | 12.178 | 1.00 | 17.65 |
| 856 | N | ARG | A | 104 | −0.729 | 20.197 | 12.573 | 1.00 | 15.77 |
| 857 | CA | ARG | A | 104 | −0.594 | 20.314 | 14.014 | 1.00 | 16.61 |
| 858 | CB | ARG | A | 104 | −1.596 | 19.401 | 14.727 | 1.00 | 19.43 |
| 859 | CG | ARG | A | 104 | −1.319 | 19.323 | 16.240 | 1.00 | 22.08 |
| 860 | CD | ARG | A | 104 | −2.071 | 18.220 | 17.000 | 1.00 | 24.56 |
| 861 | NE | ARG | A | 104 | −1.669 | 18.268 | 18.403 | 1.00 | 25.94 |
| 862 | CZ | ARG | A | 104 | −1.959 | 17.344 | 19.317 | 1.00 | 26.63 |
| 863 | NH1 | ARG | A | 104 | −2.663 | 16.263 | 18.993 | 1.00 | 25.41 |
| 864 | NH2 | ARG | A | 104 | −1.546 | 17.517 | 20.571 | 1.00 | 27.03 |
| 865 | C | ARG | A | 104 | 0.819 | 19.925 | 14.445 | 1.00 | 16.10 |
| 866 | O | ARG | A | 104 | 1.430 | 18.999 | 13.904 | 1.00 | 16.54 |
| 867 | N | VAL | A | 105 | 1.366 | 20.656 | 15.408 | 1.00 | 15.36 |
| 868 | CA | VAL | A | 105 | 2.664 | 20.319 | 15.945 | 1.00 | 15.62 |
| 869 | CB | VAL | A | 105 | 3.777 | 21.274 | 15.456 | 1.00 | 15.43 |
| 870 | CG1 | VAL | A | 105 | 3.919 | 21.113 | 13.935 | 1.00 | 18.41 |
| 871 | CG2 | VAL | A | 105 | 3.468 | 22.718 | 15.822 | 1.00 | 18.06 |
| 872 | C | VAL | A | 105 | 2.456 | 20.380 | 17.449 | 1.00 | 13.59 |
| 873 | O | VAL | A | 105 | 1.448 | 20.879 | 17.907 | 1.00 | 16.69 |
| 874 | N | PRO | A | 106 | 3.403 | 19.857 | 18.232 | 1.00 | 18.80 |
| 875 | CD | PRO | A | 106 | 4.722 | 19.326 | 17.864 | 1.00 | 18.82 |
| 876 | CA | PRO | A | 106 | 3.237 | 19.878 | 19.690 | 1.00 | 19.55 |
| 877 | CB | PRO | A | 106 | 4.566 | 19.315 | 20.199 | 1.00 | 19.56 |
| 878 | CG | PRO | A | 106 | 5.054 | 18.494 | 19.075 | 1.00 | 20.35 |
| 879 | C | PRO | A | 106 | 2.935 | 21.230 | 20.301 | 1.00 | 19.68 |
| 880 | O | PRO | A | 106 | 3.425 | 22.249 | 19.832 | 1.00 | 21.59 |
| 881 | N | GLY | A | 107 | 2.128 | 21.233 | 21.354 | 1.00 | 20.66 |
| 882 | CA | GLY | A | 107 | 1.824 | 22.493 | 22.005 | 1.00 | 22.99 |
| 883 | C | GLY | A | 107 | 0.370 | 22.891 | 21.988 | 1.00 | 22.95 |
| 884 | O | GLY | A | 107 | −0.479 | 22.213 | 21.413 | 1.00 | 26.62 |
| 885 | N | HIS | A | 108 | 0.070 | 24.011 | 22.633 | 1.00 | 23.86 |
| 886 | CA | HIS | A | 108 | −1.300 | 24.476 | 22.700 | 1.00 | 23.63 |
| 887 | CB | HIS | A | 108 | −1.487 | 25.307 | 23.972 | 1.00 | 26.13 |
| 888 | CG | HIS | A | 108 | −2.908 | 25.687 | 24.242 | 1.00 | 30.80 |
| 889 | CD2 | HIS | A | 108 | −4.069 | 25.041 | 23.979 | 1.00 | 32.61 |
| 890 | ND1 | HIS | A | 108 | −3.260 | 26.883 | 24.834 | 1.00 | 34.25 |
| 891 | CE1 | HIS | A | 108 | −4.578 | 26.960 | 24.910 | 1.00 | 33.96 |
| 892 | NE2 | HIS | A | 108 | −5.091 | 25.856 | 24.398 | 1.00 | 34.63 |
| 893 | C | HIS | A | 108 | −1.648 | 25.310 | 21.470 | 1.00 | 23.33 |
| 894 | O | HIS | A | 108 | −1.026 | 26.348 | 21.245 | 1.00 | 20.80 |
| 895 | N | ASN | A | 109 | −2.622 | 24.853 | 20.677 | 1.00 | 20.80 |
| 896 | CA | ASN | A | 109 | −3.054 | 25.580 | 19.477 | 1.00 | 21.61 |
| 897 | CB | ASN | A | 109 | −3.682 | 26.932 | 19.864 | 1.00 | 24.97 |
| 898 | CG | ASN | A | 109 | −5.098 | 26.811 | 20.432 | 1.00 | 29.39 |
| 899 | OD1 | ASN | A | 109 | −5.503 | 25.764 | 20.923 | 1.00 | 30.70 |
| 900 | ND2 | ASN | A | 109 | −5.846 | 27.911 | 20.380 | 1.00 | 30.97 |
| 901 | C | ASN | A | 109 | −1.883 | 25.858 | 18.521 | 1.00 | 20.37 |
| 902 | O | ASN | A | 109 | −1.882 | 26.890 | 17.835 | 1.00 | 19.86 |
| 903 | N | LYS | A | 110 | −0.898 | 24.965 | 18.467 | 1.00 | 18.56 |
| 904 | CA | LYS | A | 110 | 0.254 | 25.178 | 17.592 | 1.00 | 16.64 |
| 905 | CB | LYS | A | 110 | 1.553 | 24.723 | 18.265 | 1.00 | 16.98 |
| 906 | CG | LYS | A | 110 | 2.094 | 25.644 | 19.356 | 1.00 | 24.91 |
| 907 | CD | LYS | A | 110 | 3.613 | 25.527 | 19.414 | 1.00 | 27.43 |
| 908 | CE | LYS | A | 110 | 4.203 | 26.319 | 20.567 | 1.00 | 30.69 |
| 909 | NZ | LYS | A | 110 | 4.255 | 27.769 | 20.304 | 1.00 | 33.04 |
| 910 | C | LYS | A | 110 | 0.143 | 24.464 | 16.267 | 1.00 | 15.55 |
| 911 | O | LYS | A | 110 | −0.298 | 23.306 | 16.196 | 1.00 | 15.29 |
| 912 | N | MET | A | 111 | 0.534 | 25.160 | 15.205 | 1.00 | 14.18 |
| 913 | CA | MET | A | 111 | 0.507 | 24.538 | 13.896 | 1.00 | 14.57 |
| 914 | CB | MET | A | 111 | −0.891 | 24.618 | 13.321 | 1.00 | 16.69 |
| 915 | CG | MET | A | 111 | −1.287 | 26.045 | 13.036 | 1.00 | 15.81 |
| 916 | SD | MET | A | 111 | −2.929 | 26.118 | 12.289 | 1.00 | 21.78 |
| 917 | CE | MET | A | 111 | −3.925 | 25.783 | 13.747 | 1.00 | 21.24 |
| 918 | C | MET | A | 111 | 1.467 | 25.206 | 12.929 | 1.00 | 14.54 |
| 919 | O | MET | A | 111 | 1.996 | 26.286 | 13.192 | 1.00 | 14.18 |
| 920 | N | GLU | A | 112 | 1.690 | 24.531 | 11.819 | 1.00 | 12.42 |
| 921 | CA | GLU | A | 112 | 2.517 | 25.055 | 10.750 | 1.00 | 12.11 |
| 922 | CB | GLU | A | 112 | 3.669 | 24.110 | 10.393 | 1.00 | 12.57 |
| 923 | CG | GLU | A | 112 | 4.665 | 23.835 | 11.525 | 1.00 | 14.68 |
| 924 | CD | GLU | A | 112 | 5.526 | 22.602 | 11.248 | 1.00 | 17.35 |
| 925 | OE1 | GLU | A | 112 | 5.104 | 21.746 | 10.444 | 1.00 | 17.25 |
| 926 | OE2 | GLU | A | 112 | 6.614 | 22.487 | 11.846 | 1.00 | 19.59 |
| 927 | C | GLU | A | 112 | 1.602 | 25.147 | 9.551 | 1.00 | 12.78 |
| 928 | O | GLU | A | 112 | 0.516 | 24.552 | 9.540 | 1.00 | 13.84 |
| 929 | N | PHE | A | 113 | 2.051 | 25.898 | 8.554 | 1.00 | 11.10 |
| 930 | CA | PHE | A | 113 | 1.302 | 26.052 | 7.308 | 1.00 | 11.96 |
| 931 | CB | PHE | A | 113 | 0.874 | 27.502 | 7.098 | 1.00 | 11.23 |
| 932 | CG | PHE | A | 113 | −0.127 | 27.972 | 8.088 | 1.00 | 11.83 |
| 933 | CD1 | PHE | A | 113 | 0.272 | 28.473 | 9.311 | 1.00 | 12.39 |
| 934 | CD2 | PHE | A | 113 | −1.480 | 27.906 | 7.801 | 1.00 | 13.91 |
| 935 | CE1 | PHE | A | 113 | −0.657 | 28.910 | 10.238 | 1.00 | 14.18 |
| 936 | CE2 | PHE | A | 113 | −2.413 | 28.338 | 8.719 | 1.00 | 15.19 |
| 937 | CZ | PHE | A | 113 | −2.002 | 28.843 | 9.938 | 1.00 | 16.27 |
| 938 | C | PHE | A | 113 | 2.234 | 25.649 | 6.198 | 1.00 | 10.30 |
| 939 | O | PHE | A | 113 | 3.173 | 26.380 | 5.872 | 1.00 | 12.19 |
| 940 | N | GLU | A | 114 | 1.981 | 24.484 | 5.613 | 1.00 | 10.85 |
| 941 | CA | GLU | A | 114 | 2.830 | 24.000 | 4.536 | 1.00 | 11.42 |
| 942 | CB | GLU | A | 114 | 2.960 | 22.483 | 4.616 | 1.00 | 14.13 |
| 943 | CG | GLU | A | 114 | 4.017 | 21.966 | 3.647 | 1.00 | 14.86 |
| 944 | CD | GLU | A | 114 | 4.349 | 20.505 | 3.830 | 1.00 | 18.25 |
| 945 | OE1 | GLU | A | 114 | 3.732 | 19.820 | 4.686 | 1.00 | 19.69 |
| 946 | OE2 | GLU | A | 114 | 5.256 | 20.043 | 3.096 | 1.00 | 19.74 |
| 947 | C | GLU | A | 114 | 2.256 | 24.382 | 3.174 | 1.00 | 11.11 |
| 948 | O | GLU | A | 114 | 1.082 | 24.170 | 2.899 | 1.00 | 12.56 |
| 949 | N | SER | A | 115 | 3.101 | 24.954 | 2.335 | 1.00 | 11.88 |
| 950 | CA | SER | A | 115 | 2.655 | 25.349 | 1.008 | 1.00 | 12.18 |
| 951 | CB | SER | A | 115 | 3.779 | 26.089 | 0.303 | 1.00 | 13.90 |
| 952 | OG | SER | A | 115 | 3.415 | 26.376 | −1.036 | 1.00 | 14.01 |
| 953 | C | SER | A | 115 | 2.208 | 24.163 | 0.144 | 1.00 | 14.22 |
| 954 | O | SER | A | 115 | 2.892 | 23.141 | 0.080 | 1.00 | 14.80 |
| 955 | N | SER | A | 116 | 1.057 | 24.286 | −0.520 | 1.00 | 12.54 |
| 956 | CA | SER | A | 116 | 0.607 | 23.207 | −1.395 | 1.00 | 13.95 |
| 957 | CB | SER | A | 116 | −0.889 | 23.335 | −1.709 | 1.00 | 15.49 |
| 958 | OG | SER | A | 116 | −1.114 | 24.396 | −2.605 | 1.00 | 21.00 |
| 959 | C | SER | A | 116 | 1.410 | 23.256 | −2.689 | 1.00 | 14.78 |
| 960 | O | SER | A | 116 | 1.620 | 22.222 | −3.323 | 1.00 | 20.48 |
| 961 | N | LEU | A | 117 | 1.871 | 24.443 | −3.068 | 1.00 | 15.76 |
| 962 | CA | LEU | A | 117 | 2.665 | 24.610 | −4.296 | 1.00 | 14.89 |
| 963 | CB | LEU | A | 117 | 2.527 | 26.054 | −4.778 | 1.00 | 17.05 |
| 964 | CG | LEU | A | 117 | 3.259 | 26.411 | −6.073 | 1.00 | 18.25 |
| 965 | CD1 | LEU | A | 117 | 2.711 | 25.548 | −7.205 | 1.00 | 20.93 |
| 966 | CD2 | LEU | A | 117 | 3.075 | 27.907 | −6.367 | 1.00 | 19.17 |
| 967 | C | LEU | A | 117 | 4.148 | 24.232 | −4.108 | 1.00 | 16.33 |
| 968 | O | LEU | A | 117 | 4.785 | 23.640 | −5.000 | 1.00 | 19.22 |
| 969 | N | TYR | A | 118 | 4.698 | 24.568 | −2.951 | 1.00 | 15.94 |
| 970 | CA | TYR | A | 118 | 6.089 | 24.258 | −2.632 | 1.00 | 16.21 |
| 971 | CB | TYR | A | 118 | 6.843 | 25.549 | −2.311 | 1.00 | 17.11 |
| 972 | CG | TYR | A | 118 | 6.753 | 26.539 | −3.451 | 1.00 | 17.91 |
| 973 | CD1 | TYR | A | 118 | 7.274 | 26.224 | −4.706 | 1.00 | 20.29 |
| 974 | CE1 | TYR | A | 118 | 7.122 | 27.096 | −5.790 | 1.00 | 19.88 |
| 975 | CD2 | TYR | A | 118 | 6.082 | 27.749 | −3.301 | 1.00 | 18.50 |
| 976 | CE2 | TYR | A | 118 | 5.924 | 28.629 | −4.374 | 1.00 | 20.19 |
| 977 | CZ | TYR | A | 118 | 6.445 | 28.293 | −5.617 | 1.00 | 20.90 |
| 978 | OH | TYR | A | 118 | 6.260 | 29.144 | −6.684 | 1.00 | 20.87 |
| 979 | C | TYR | A | 118 | 6.141 | 23.316 | −1.445 | 1.00 | 16.88 |
| 980 | O | TYR | A | 118 | 6.317 | 23.742 | −0.302 | 1.00 | 15.27 |
| 981 | N | GLU | A | 119 | 6.006 | 22.023 | −1.714 | 1.00 | 16.33 |
| 982 | CA | GLU | A | 119 | 6.028 | 21.036 | −0.648 | 1.00 | 18.42 |
| 983 | CB | GLU | A | 119 | 5.941 | 19.621 | −1.231 | 1.00 | 21.75 |
| 984 | CG | GLU | A | 119 | 4.638 | 19.358 | −1.976 | 1.00 | 30.12 |
| 985 | CD | GLU | A | 119 | 4.572 | 17.950 | −2.549 | 1.00 | 34.08 |
| 986 | OE1 | GLU | A | 119 | 4.853 | 17.007 | −1.781 | 1.00 | 37.54 |
| 987 | OE2 | GLU | A | 119 | 4.237 | 17.794 | −3.754 | 1.00 | 38.83 |
| 988 | C | GLU | A | 119 | 7.319 | 21.204 | 0.125 | 1.00 | 15.05 |
| 989 | O | GLU | A | 119 | 8.367 | 21.498 | −0.451 | 1.00 | 17.32 |
| 990 | N | GLY | A | 120 | 7.244 | 21.047 | 1.440 | 1.00 | 16.26 |
| 991 | CA | GLY | A | 120 | 8.446 | 21.183 | 2.236 | 1.00 | 17.41 |
| 992 | C | GLY | A | 120 | 8.806 | 22.617 | 2.581 | 1.00 | 15.81 |
| 993 | O | GLY | A | 120 | 9.816 | 22.868 | 3.237 | 1.00 | 17.99 |
| 994 | N | HIS | A | 121 | 7.998 | 23.570 | 2.116 | 1.00 | 15.30 |
| 995 | CA | HIS | A | 121 | 8.227 | 24.976 | 2.422 | 1.00 | 13.61 |
| 996 | CB | HIS | A | 121 | 8.247 | 25.828 | 1.160 | 1.00 | 17.73 |
| 997 | CG | HIS | A | 121 | 9.401 | 25.528 | 0.257 | 1.00 | 17.99 |
| 998 | CD2 | HIS | A | 121 | 9.784 | 24.378 | −0.346 | 1.00 | 18.39 |
| 999 | ND1 | HIS | A | 121 | 10.340 | 26.474 | −0.087 | 1.00 | 21.46 |
| 1000 | CE1 | HIS | A | 121 | 11.255 | 25.921 | −0.865 | 1.00 | 19.82 |
| 1001 | NE2 | HIS | A | 121 | 10.941 | 24.650 | −1.038 | 1.00 | 19.41 |
| 1002 | C | HIS | A | 121 | 7.058 | 25.379 | 3.294 | 1.00 | 14.70 |
| 1003 | O | HIS | A | 121 | 5.921 | 24.954 | 3.055 | 1.00 | 14.66 |
| 1004 | N | PHE | A | 122 | 7.345 | 26.213 | 4.285 | 1.00 | 12.51 |
| 1005 | CA | PHE | A | 122 | 6.329 | 26.597 | 5.247 | 1.00 | 12.15 |
| 1006 | CB | PHE | A | 122 | 6.619 | 25.953 | 6.607 | 1.00 | 13.26 |
| 1007 | CG | PHE | A | 122 | 6.663 | 24.467 | 6.577 | 1.00 | 14.18 |
| 1008 | CD1 | PHE | A | 122 | 7.765 | 23.792 | 6.050 | 1.00 | 13.89 |
| 1009 | CD2 | PHE | A | 122 | 5.597 | 23.745 | 7.074 | 1.00 | 14.29 |
| 1010 | CE1 | PHE | A | 122 | 7.790 | 22.388 | 6.020 | 1.00 | 14.70 |
| 1011 | CE2 | PHE | A | 122 | 5.610 | 22.344 | 7.055 | 1.00 | 13.73 |
| 1012 | CZ | PHE | A | 122 | 6.709 | 21.674 | 6.524 | 1.00 | 14.90 |
| 1013 | C | PHE | A | 122 | 6.323 | 28.084 | 5.486 | 1.00 | 12.57 |
| 1014 | O | PHE | A | 122 | 7.314 | 28.756 | 5.254 | 1.00 | 13.14 |
| 1015 | N | LEU | A | 123 | 5.186 | 28.600 | 5.952 | 1.00 | 10.71 |
| 1016 | CA | LEU | A | 123 | 5.152 | 29.996 | 6.358 | 1.00 | 11.63 |
| 1017 | CB | LEU | A | 123 | 3.748 | 30.388 | 6.791 | 1.00 | 9.10 |
| 1018 | CG | LEU | A | 123 | 2.795 | 30.423 | 5.609 | 1.00 | 10.54 |
| 1019 | CD1 | LEU | A | 123 | 1.429 | 30.905 | 6.117 | 1.00 | 11.15 |
| 1020 | CD2 | LEU | A | 123 | 3.318 | 31.377 | 4.527 | 1.00 | 11.37 |
| 1021 | C | LEU | A | 123 | 6.081 | 30.076 | 7.577 | 1.00 | 11.96 |
| 1022 | O | LEU | A | 123 | 6.198 | 29.125 | 8.366 | 1.00 | 12.05 |
| 1023 | N | ALA | A | 124 | 6.720 | 31.225 | 7.753 | 1.00 | 12.85 |
| 1024 | CA | ALA | A | 124 | 7.622 | 31.365 | 8.884 | 1.00 | 11.53 |
| 1025 | CB | ALA | A | 124 | 8.980 | 30.738 | 8.550 | 1.00 | 13.24 |
| 1026 | C | ALA | A | 124 | 7.818 | 32.820 | 9.198 | 1.00 | 12.94 |
| 1027 | O | ALA | A | 124 | 7.524 | 33.691 | 8.380 | 1.00 | 13.23 |
| 1028 | N | CYS | A | 125 | 8.322 | 33.067 | 10.402 | 1.00 | 13.26 |
| 1029 | CA | CYS | A | 125 | 8.644 | 34.415 | 10.812 | 1.00 | 13.48 |
| 1030 | CB | CYS | A | 125 | 8.251 | 34.667 | 12.265 | 1.00 | 13.20 |
| 1031 | SG | CYS | A | 125 | 8.781 | 36.300 | 12.818 | 1.00 | 19.51 |
| 1032 | C | CYS | A | 125 | 10.157 | 34.519 | 10.681 | 1.00 | 15.89 |
| 1033 | O | CYS | A | 125 | 10.887 | 33.580 | 11.020 | 1.00 | 18.43 |
| 1034 | N | GLN | A | 126 | 10.630 | 35.640 | 10.151 | 1.00 | 16.06 |
| 1035 | CA | GLN | A | 126 | 12.069 | 35.842 | 10.012 | 1.00 | 17.60 |
| 1036 | CB | GLN | A | 126 | 12.539 | 35.509 | 8.593 | 1.00 | 20.69 |
| 1037 | CG | GLN | A | 126 | 14.052 | 35.634 | 8.399 | 1.00 | 24.00 |
| 1038 | CD | AGLN | A | 126 | 14.759 | 34.337 | 8.674 | 0.50 | 22.65 |
| 1039 | CD | BGLN | A | 126 | 14.514 | 35.169 | 7.021 | 0.50 | 23.59 |
| 1040 | OE1 | AGLN | A | 126 | 14.583 | 33.359 | 7.948 | 0.50 | 26.30 |
| 1041 | OE1 | BGLN | A | 126 | 14.245 | 35.819 | 6.009 | 0.50 | 25.95 |
| 1042 | NE2 | AGLN | A | 126 | 15.564 | 34.313 | 9.725 | 0.50 | 27.75 |
| 1043 | NE2 | BGLN | A | 126 | 15.200 | 34.033 | 6.976 | 0.50 | 24.53 |
| 1044 | C | GLN | A | 126 | 12.382 | 37.289 | 10.304 | 1.00 | 18.81 |
| 1045 | O | GLN | A | 126 | 11.760 | 38.186 | 9.752 | 1.00 | 19.93 |
| 1046 | N | LYS | A | 127 | 13.362 | 37.515 | 11.173 | 1.00 | 20.55 |
| 1047 | CA | LYS | A | 127 | 13.748 | 38.879 | 11.491 | 1.00 | 23.77 |
| 1048 | CB | LYS | A | 127 | 14.567 | 38.925 | 12.779 | 1.00 | 24.21 |
| 1049 | CG | LYS | A | 127 | 14.802 | 40.337 | 13.301 | 1.00 | 27.87 |
| 1050 | CD | LYS | A | 127 | 15.675 | 40.321 | 14.540 | 1.00 | 29.02 |
| 1051 | CE | LYS | A | 127 | 15.836 | 41.713 | 15.144 | 1.00 | 31.54 |
| 1052 | NZ | LYS | A | 127 | 16.437 | 41.644 | 16.509 | 1.00 | 33.62 |
| 1053 | C | LYS | A | 127 | 14.582 | 39.402 | 10.334 | 1.00 | 25.42 |
| 1054 | O | LYS | A | 127 | 15.509 | 38.743 | 9.887 | 1.00 | 25.74 |
| 1055 | N | GLU | A | 128 | 14.220 | 40.571 | 9.826 | 1.00 | 25.95 |
| 1056 | CA | GLU | A | 128 | 14.966 | 41.192 | 8.747 | 1.00 | 29.30 |
| 1057 | CB | GLU | A | 128 | 14.130 | 41.291 | 7.480 | 1.00 | 31.49 |
| 1058 | CG | GLU | A | 128 | 14.964 | 41.545 | 6.227 | 1.00 | 35.73 |
| 1059 | CD | GLU | A | 128 | 14.170 | 42.212 | 5.126 | 1.00 | 38.50 |
| 1060 | OE1 | GLU | A | 128 | 13.862 | 43.417 | 5.264 | 1.00 | 41.50 |
| 1061 | OE2 | GLU | A | 128 | 13.843 | 41.542 | 4.122 | 1.00 | 41.96 |
| 1062 | C | GLU | A | 128 | 15.254 | 42.576 | 9.288 | 1.00 | 31.50 |
| 1063 | O | GLU | A | 128 | 14.342 | 43.397 | 9.426 | 1.00 | 32.21 |
| 1064 | N | ASP | A | 129 | 16.518 | 42.829 | 9.613 | 1.00 | 32.76 |
| 1065 | CA | ASP | A | 129 | 16.913 | 44.106 | 10.181 | 1.00 | 35.20 |
| 1066 | CB | ASP | A | 129 | 16.624 | 45.235 | 9.190 | 1.00 | 37.54 |
| 1067 | CG | ASP | A | 129 | 17.578 | 45.205 | 7.997 | 1.00 | 39.65 |
| 1068 | OD1 | ASP | A | 129 | 18.813 | 45.254 | 8.207 | 1.00 | 40.49 |
| 1069 | OD2 | ASP | A | 129 | 17.105 | 45.111 | 6.846 | 1.00 | 43.03 |
| 1070 | C | ASP | A | 129 | 16.165 | 44.273 | 11.499 | 1.00 | 34.76 |
| 1071 | O | ASP | A | 129 | 16.418 | 43.520 | 12.436 | 1.00 | 36.76 |
| 1072 | N | ASP | A | 130 | 15.253 | 45.222 | 11.623 | 1.00 | 34.20 |
| 1073 | CA | ASP | A | 130 | 14.577 | 45.313 | 12.911 | 1.00 | 32.98 |
| 1074 | CB | ASP | A | 130 | 14.775 | 46.705 | 13.522 | 1.00 | 36.86 |
| 1075 | CG | ASP | A | 130 | 16.115 | 46.829 | 14.264 | 1.00 | 38.69 |
| 1076 | OD1 | ASP | A | 130 | 16.259 | 46.203 | 15.340 | 1.00 | 39.67 |
| 1077 | OD2 | ASP | A | 130 | 17.037 | 47.534 | 13.774 | 1.00 | 41.31 |
| 1078 | C | ASP | A | 130 | 13.102 | 44.921 | 12.852 | 1.00 | 31.57 |
| 1079 | O | ASP | A | 130 | 12.378 | 45.027 | 13.845 | 1.00 | 32.43 |
| 1080 | N | ALA | A | 131 | 12.679 | 44.430 | 11.692 | 1.00 | 27.42 |
| 1081 | CA | ALA | A | 131 | 11.302 | 44.006 | 11.488 | 1.00 | 23.99 |
| 1082 | CB | ALA | A | 131 | 10.819 | 44.445 | 10.117 | 1.00 | 24.71 |
| 1083 | C | ALA | A | 131 | 11.203 | 42.492 | 11.586 | 1.00 | 22.78 |
| 1084 | O | ALA | A | 131 | 12.187 | 41.785 | 11.419 | 1.00 | 24.27 |
| 1085 | N | PHE | A | 132 | 10.008 | 41.996 | 11.869 | 1.00 | 19.22 |
| 1086 | CA | PHE | A | 132 | 9.791 | 40.551 | 11.934 | 1.00 | 17.56 |
| 1087 | CB | PHE | A | 132 | 9.230 | 40.191 | 13.297 | 1.00 | 18.03 |
| 1088 | CG | PHE | A | 132 | 10.232 | 40.373 | 14.391 | 1.00 | 22.00 |
| 1089 | CD1 | PHE | A | 132 | 11.237 | 39.434 | 14.584 | 1.00 | 23.59 |
| 1090 | CD2 | PHE | A | 132 | 10.232 | 41.526 | 15.163 | 1.00 | 22.92 |
| 1091 | CE1 | PHE | A | 132 | 12.245 | 39.642 | 15.539 | 1.00 | 25.47 |
| 1092 | CE2 | PHE | A | 132 | 11.232 | 41.742 | 16.115 | 1.00 | 23.24 |
| 1093 | CZ | PHE | A | 132 | 12.233 | 40.798 | 16.298 | 1.00 | 24.74 |
| 1094 | C | PHE | A | 132 | 8.826 | 40.277 | 10.796 | 1.00 | 16.02 |
| 1095 | O | PHE | A | 132 | 7.675 | 40.683 | 10.842 | 1.00 | 17.63 |
| 1096 | N | LYS | A | 133 | 9.315 | 39.592 | 9.775 | 1.00 | 15.51 |
| 1097 | CA | LYS | A | 133 | 8.522 | 39.370 | 8.577 | 1.00 | 15.56 |
| 1098 | CB | LYS | A | 133 | 9.380 | 39.726 | 7.358 | 1.00 | 21.68 |
| 1099 | CG | LYS | A | 133 | 10.022 | 41.103 | 7.414 | 1.00 | 23.79 |
| 1100 | CD | LYS | A | 133 | 8.984 | 42.198 | 7.316 | 1.00 | 28.93 |
| 1101 | CE | LYS | A | 133 | 9.579 | 43.556 | 6.912 | 1.00 | 32.70 |
| 1102 | NZ | LYS | A | 133 | 10.002 | 43.637 | 5.476 | 1.00 | 33.12 |
| 1103 | C | LYS | A | 133 | 7.956 | 37.980 | 8.383 | 1.00 | 13.95 |
| 1104 | O | LYS | A | 133 | 8.446 | 37.008 | 8.956 | 1.00 | 14.81 |
| 1105 | N | LEU | A | 134 | 6.899 | 37.898 | 7.576 | 1.00 | 13.83 |
| 1106 | CA | LEU | A | 134 | 6.293 | 36.614 | 7.240 | 1.00 | 13.03 |
| 1107 | CB | LEU | A | 134 | 4.805 | 36.787 | 7.019 | 1.00 | 13.18 |
| 1108 | CG | LEU | A | 134 | 4.051 | 35.543 | 6.569 | 1.00 | 12.67 |
| 1109 | CD1 | LEU | A | 134 | 4.055 | 34.488 | 7.705 | 1.00 | 13.20 |
| 1110 | CD2 | LEU | A | 134 | 2.625 | 35.935 | 6.232 | 1.00 | 12.70 |
| 1111 | C | LEU | A | 134 | 6.956 | 36.197 | 5.943 | 1.00 | 13.54 |
| 1112 | O | LEU | A | 134 | 6.921 | 36.940 | 4.963 | 1.00 | 14.23 |
| 1113 | N | ILE | A | 135 | 7.570 | 35.023 | 5.932 | 1.00 | 12.99 |
| 1114 | CA | ILE | A | 135 | 8.240 | 34.523 | 4.724 | 1.00 | 13.65 |
| 1115 | CB | ILE | A | 135 | 9.786 | 34.572 | 4.863 | 1.00 | 14.46 |
| 1116 | CG2 | ILE | A | 135 | 10.251 | 36.027 | 4.996 | 1.00 | 17.46 |
| 1117 | CG1 | ILE | A | 135 | 10.214 | 33.705 | 6.053 | 1.00 | 15.82 |
| 1118 | CD1 | ILE | A | 135 | 11.691 | 33.339 | 6.042 | 1.00 | 18.39 |
| 1119 | C | ILE | A | 135 | 7.863 | 33.064 | 4.479 | 1.00 | 13.24 |
| 1120 | O | ILE | A | 135 | 7.119 | 32.475 | 5.265 | 1.00 | 13.19 |
| 1121 | N | LEU | A | 136 | 8.359 | 32.496 | 3.382 | 1.00 | 12.56 |
| 1122 | CA | LEU | A | 136 | 8.147 | 31.094 | 3.088 | 1.00 | 12.33 |
| 1123 | CB | LEU | A | 136 | 7.558 | 30.874 | 1.687 | 1.00 | 14.97 |
| 1124 | CG | LEU | A | 136 | 7.235 | 29.400 | 1.401 | 1.00 | 14.23 |
| 1125 | CD1 | LEU | A | 136 | 5.865 | 29.098 | 2.028 | 1.00 | 13.48 |
| 1126 | CD2 | LEU | A | 136 | 7.214 | 29.112 | −0.133 | 1.00 | 13.37 |
| 1127 | C | LEU | A | 136 | 9.557 | 30.511 | 3.104 | 1.00 | 14.28 |
| 1128 | O | LEU | A | 136 | 10.451 | 31.056 | 2.467 | 1.00 | 16.88 |
| 1129 | N | LYS | A | 137 | 9.750 | 29.402 | 3.802 | 1.00 | 13.54 |
| 1130 | CA | LYS | A | 137 | 11.071 | 28.794 | 3.858 | 1.00 | 14.56 |
| 1131 | CB | LYS | A | 137 | 11.890 | 29.414 | 4.996 | 1.00 | 15.95 |
| 1132 | CG | LYS | A | 137 | 11.371 | 29.062 | 6.381 | 1.00 | 18.25 |
| 1133 | CD | LYS | A | 137 | 12.186 | 29.691 | 7.486 | 1.00 | 22.46 |
| 1134 | CE | LYS | A | 137 | 13.067 | 28.690 | 8.193 | 1.00 | 21.75 |
| 1135 | NZ | LYS | A | 137 | 13.728 | 29.370 | 9.346 | 1.00 | 17.93 |
| 1136 | C | LYS | A | 137 | 11.001 | 27.314 | 4.132 | 1.00 | 14.87 |
| 1137 | O | LYS | A | 137 | 9.989 | 26.798 | 4.593 | 1.00 | 14.80 |
| 1138 | N | LYS | A | 138 | 12.101 | 26.630 | 3.838 | 1.00 | 16.07 |
| 1139 | CA | LYS | A | 138 | 12.216 | 25.231 | 4.200 | 1.00 | 16.43 |
| 1140 | CB | LYS | A | 138 | 13.358 | 24.556 | 3.454 | 1.00 | 18.90 |
| 1141 | CG | LYS | A | 138 | 13.110 | 24.365 | 1.987 | 1.00 | 20.88 |
| 1142 | CD | LYS | A | 138 | 14.012 | 23.255 | 1.485 | 1.00 | 25.69 |
| 1143 | CE | LYS | A | 138 | 13.894 | 23.048 | 0.008 | 1.00 | 26.71 |
| 1144 | NZ | LYS | A | 138 | 14.451 | 21.707 | −0.367 | 1.00 | 28.61 |
| 1145 | C | LYS | A | 138 | 12.628 | 25.336 | 5.670 | 1.00 | 15.78 |
| 1146 | O | LYS | A | 138 | 13.155 | 26.367 | 6.100 | 1.00 | 18.59 |
| 1147 | N | LYS | A | 139 | 12.389 | 24.286 | 6.444 | 1.00 | 17.51 |
| 1148 | CA | LYS | A | 139 | 12.795 | 24.285 | 7.848 | 1.00 | 16.96 |
| 1149 | CB | LYS | A | 139 | 12.307 | 23.004 | 8.527 | 1.00 | 18.35 |
| 1150 | CG | LYS | A | 139 | 10.804 | 22.911 | 8.742 | 1.00 | 19.71 |
| 1151 | CD | LYS | A | 139 | 10.527 | 21.512 | 9.258 | 1.00 | 23.58 |
| 1152 | CE | LYS | A | 139 | 9.066 | 21.209 | 9.534 | 1.00 | 26.29 |
| 1153 | NZ | LYS | A | 139 | 8.954 | 19.737 | 9.843 | 1.00 | 25.65 |
| 1154 | C | LYS | A | 139 | 14.330 | 24.333 | 7.881 | 1.00 | 17.92 |
| 1155 | O | LYS | A | 139 | 14.973 | 23.710 | 7.033 | 1.00 | 19.75 |
| 1156 | N | ASP | A | 140 | 14.911 | 25.091 | 8.811 | 1.00 | 19.30 |
| 1157 | CA | ASP | A | 140 | 16.381 | 25.156 | 8.910 | 1.00 | 20.30 |
| 1158 | CB | ASP | A | 140 | 16.866 | 26.140 | 9.973 | 1.00 | 20.83 |
| 1159 | CG | ASP | A | 140 | 16.617 | 27.566 | 9.612 | 1.00 | 20.94 |
| 1160 | OD1 | ASP | A | 140 | 16.334 | 27.861 | 8.432 | 1.00 | 22.64 |
| 1161 | OD2 | ASP | A | 140 | 16.724 | 28.403 | 10.532 | 1.00 | 25.18 |
| 1162 | C | ASP | A | 140 | 16.853 | 23.797 | 9.344 | 1.00 | 22.77 |
| 1163 | O | ASP | A | 140 | 17.929 | 23.341 | 8.964 | 1.00 | 23.14 |
| 1164 | N | GLU | A | 141 | 16.056 | 23.179 | 10.198 | 1.00 | 22.47 |
| 1165 | CA | GLU | A | 141 | 16.372 | 21.859 | 10.676 | 1.00 | 26.83 |
| 1166 | CB | GLU | A | 141 | 17.531 | 21.903 | 11.676 | 1.00 | 28.35 |
| 1167 | CG | GLU | A | 141 | 17.193 | 22.548 | 13.013 | 1.00 | 29.02 |
| 1168 | CD | GLU | A | 141 | 17.240 | 24.067 | 12.973 | 1.00 | 32.41 |
| 1169 | OE1 | GLU | A | 141 | 18.244 | 24.623 | 12.472 | 1.00 | 32.00 |
| 1170 | OE2 | GLU | A | 141 | 16.277 | 24.703 | 13.453 | 1.00 | 33.91 |
| 1171 | C | GLU | A | 141 | 15.143 | 21.326 | 11.352 | 1.00 | 28.16 |
| 1172 | O | GLU | A | 141 | 14.141 | 22.025 | 11.514 | 1.00 | 26.16 |
| 1173 | N | ASN | A | 142 | 15.215 | 20.057 | 11.718 | 1.00 | 30.27 |
| 1174 | CA | ASN | A | 142 | 14.126 | 19.423 | 12.413 | 1.00 | 31.97 |
| 1175 | CB | ASN | A | 142 | 14.482 | 17.962 | 12.675 | 1.00 | 35.33 |
| 1176 | CG | ASN | A | 142 | 13.436 | 17.248 | 13.490 | 1.00 | 37.20 |
| 1177 | OD1 | ASN | A | 142 | 12.591 | 16.534 | 12.948 | 1.00 | 39.29 |
| 1178 | ND2 | ASN | A | 142 | 13.478 | 17.441 | 14.808 | 1.00 | 39.62 |
| 1179 | C | ASN | A | 142 | 14.096 | 20.186 | 13.726 | 1.00 | 33.28 |
| 1180 | O | ASN | A | 142 | 15.108 | 20.247 | 14.434 | 1.00 | 34.62 |
| 1181 | N | GLY | A | 143 | 12.965 | 20.787 | 14.057 | 1.00 | 33.41 |
| 1182 | CA | GLY | A | 143 | 12.915 | 21.521 | 15.310 | 1.00 | 31.70 |
| 1183 | C | GLY | A | 143 | 12.959 | 23.022 | 15.122 | 1.00 | 29.85 |
| 1184 | O | GLY | A | 143 | 13.018 | 23.775 | 16.091 | 1.00 | 30.77 |
| 1185 | N | ASP | A | 144 | 12.944 | 23.454 | 13.867 | 1.00 | 25.57 |
| 1186 | CA | ASP | A | 144 | 12.944 | 24.868 | 13.544 | 1.00 | 23.35 |
| 1187 | CB | ASP | A | 144 | 13.083 | 25.060 | 12.041 | 1.00 | 22.25 |
| 1188 | CG | ASP | A | 144 | 13.166 | 26.507 | 11.655 | 1.00 | 21.13 |
| 1189 | OD1 | ASP | A | 144 | 12.927 | 27.377 | 12.526 | 1.00 | 18.37 |
| 1190 | OD2 | ASP | A | 144 | 13.473 | 26.771 | 10.480 | 1.00 | 17.30 |
| 1191 | C | ASP | A | 144 | 11.590 | 25.381 | 13.991 | 1.00 | 20.88 |
| 1192 | O | ASP | A | 144 | 10.559 | 25.071 | 13.397 | 1.00 | 22.99 |
| 1193 | N | LYS | A | 145 | 11.581 | 26.174 | 15.040 | 1.00 | 19.51 |
| 1194 | CA | LYS | A | 145 | 10.322 | 26.671 | 15.546 | 1.00 | 18.75 |
| 1195 | CB | LYS | A | 145 | 10.439 | 26.922 | 17.050 | 1.00 | 21.90 |
| 1196 | CG | LYS | A | 145 | 10.687 | 25.648 | 17.872 | 1.00 | 24.52 |
| 1197 | CD | LYS | A | 145 | 10.549 | 25.948 | 19.366 | 1.00 | 26.57 |
| 1198 | CE | LYS | A | 145 | 10.796 | 24.710 | 20.217 | 1.00 | 30.11 |
| 1199 | NZ | LYS | A | 145 | 9.742 | 23.680 | 20.087 | 1.00 | 31.97 |
| 1200 | C | LYS | A | 145 | 9.810 | 27.929 | 14.862 | 1.00 | 16.66 |
| 1201 | O | LYS | A | 145 | 8.688 | 28.369 | 15.151 | 1.00 | 16.06 |
| 1202 | N | SER | A | 146 | 10.596 | 28.498 | 13.952 | 1.00 | 15.88 |
| 1203 | CA | SER | A | 146 | 10.181 | 29.728 | 13.280 | 1.00 | 14.67 |
| 1204 | CB | SER | A | 146 | 11.328 | 30.307 | 12.437 | 1.00 | 14.91 |
| 1205 | OG | SER | A | 146 | 11.585 | 29.472 | 11.323 | 1.00 | 15.64 |
| 1206 | C | SER | A | 146 | 8.959 | 29.514 | 12.384 | 1.00 | 14.13 |
| 1207 | O | SER | A | 146 | 8.330 | 30.482 | 11.945 | 1.00 | 12.98 |
| 1208 | N | VAL | A | 147 | 8.653 | 28.252 | 12.087 | 1.00 | 13.49 |
| 1209 | CA | VAL | A | 147 | 7.501 | 27.921 | 11.250 | 1.00 | 13.71 |
| 1210 | CB | VAL | A | 147 | 7.787 | 26.739 | 10.317 | 1.00 | 11.55 |
| 1211 | CG1 | VAL | A | 147 | 8.866 | 27.099 | 9.304 | 1.00 | 14.25 |
| 1212 | CG2 | VAL | A | 147 | 8.163 | 25.536 | 11.133 | 1.00 | 15.65 |
| 1213 | C | VAL | A | 147 | 6.298 | 27.535 | 12.112 | 1.00 | 11.71 |
| 1214 | O | VAL | A | 147 | 5.258 | 27.152 | 11.590 | 1.00 | 10.63 |
| 1215 | N | MET | A | 148 | 6.424 | 27.634 | 13.435 | 1.00 | 12.60 |
| 1216 | CA | MET | A | 148 | 5.321 | 27.281 | 14.303 | 1.00 | 14.00 |
| 1217 | CB | MET | A | 148 | 5.830 | 26.516 | 15.512 | 1.00 | 14.92 |
| 1218 | CG | MET | A | 148 | 6.469 | 25.231 | 15.076 | 1.00 | 18.15 |
| 1219 | SD | MET | A | 148 | 7.076 | 24.375 | 16.500 | 1.00 | 24.45 |
| 1220 | CE | MET | A | 148 | 7.997 | 23.101 | 15.708 | 1.00 | 24.28 |
| 1221 | C | MET | A | 148 | 4.572 | 28.500 | 14.740 | 1.00 | 13.74 |
| 1222 | O | MET | A | 148 | 5.162 | 29.498 | 15.171 | 1.00 | 14.34 |
| 1223 | N | PHE | A | 149 | 3.260 | 28.418 | 14.625 | 1.00 | 12.40 |
| 1224 | CA | PHE | A | 149 | 2.380 | 29.523 | 14.980 | 1.00 | 14.24 |
| 1225 | CB | PHE | A | 149 | 1.690 | 30.066 | 13.730 | 1.00 | 13.47 |
| 1226 | CG | PHE | A | 149 | 2.630 | 30.602 | 12.727 | 1.00 | 15.00 |
| 1227 | CD1 | PHE | A | 149 | 3.129 | 31.887 | 12.863 | 1.00 | 13.31 |
| 1228 | CD2 | PHE | A | 149 | 3.063 | 29.806 | 11.673 | 1.00 | 11.42 |
| 1229 | CE1 | PHE | A | 149 | 4.058 | 32.386 | 11.964 | 1.00 | 13.16 |
| 1230 | CE2 | PHE | A | 149 | 3.989 | 30.288 | 10.769 | 1.00 | 12.94 |
| 1231 | CZ | PHE | A | 149 | 4.494 | 31.594 | 10.917 | 1.00 | 13.99 |
| 1232 | C | PHE | A | 149 | 1.282 | 29.044 | 15.888 | 1.00 | 15.18 |
| 1233 | O | PHE | A | 149 | 0.952 | 27.852 | 15.917 | 1.00 | 14.60 |
| 1234 | N | THR | A | 150 | 0.716 | 29.968 | 16.641 | 1.00 | 14.14 |
| 1235 | CA | THR | A | 150 | −0.423 | 29.612 | 17.459 | 1.00 | 16.16 |
| 1236 | CB A | THR | A | 150 | −0.225 | 29.966 | 18.939 | 0.50 | 15.90 |
| 1237 | CB B | THR | A | 150 | −0.238 | 30.083 | 18.927 | 0.50 | 17.48 |
| 1238 | OG1A | THR | A | 150 | −0.014 | 31.369 | 19.073 | 0.50 | 13.73 |
| 1239 | OG1B | THR | A | 150 | 0.849 | 29.375 | 19.533 | 0.50 | 20.99 |
| 1240 | CG2A | THR | A | 150 | 0.970 | 29.224 | 19.493 | 0.50 | 18.22 |
| 1241 | CG2B | THR | A | 150 | −1.499 | 29.845 | 19.729 | 0.50 | 19.08 |
| 1242 | C | THR | A | 150 | −1.610 | 30.374 | 16.884 | 1.00 | 16.42 |
| 1243 | O | THR | A | 150 | −1.509 | 31.572 | 16.582 | 1.00 | 16.83 |
| 1244 | N | LEU | A | 151 | −2.738 | 29.695 | 16.706 | 1.00 | 16.47 |
| 1245 | CA | LEU | A | 151 | −3.934 | 30.355 | 16.194 | 1.00 | 20.02 |
| 1246 | CB | LEU | A | 151 | −4.471 | 29.667 | 14.932 | 1.00 | 20.88 |
| 1247 | CG | LEU | A | 151 | −4.037 | 30.322 | 13.618 | 1.00 | 21.54 |
| 1248 | CD1 | LEU | A | 151 | −2.549 | 30.059 | 13.465 | 1.00 | 21.65 |
| 1249 | CD2 | LEU | A | 151 | −4.795 | 29.758 | 12.423 | 1.00 | 23.25 |
| 1250 | C | LEU | A | 151 | −5.016 | 30.333 | 17.251 | 1.00 | 24.42 |
| 1251 | O | LEU | A | 151 | −5.211 | 29.307 | 17.896 | 1.00 | 25.23 |
| 1252 | N | THR | A | 152 | −5.709 | 31.452 | 17.444 | 1.00 | 23.97 |
| 1253 | CA | THR | A | 152 | −6.790 | 31.478 | 18.433 | 1.00 | 27.51 |
| 1254 | CB | THR | A | 152 | −7.303 | 32.893 | 18.682 | 1.00 | 28.24 |
| 1255 | OG1 | THR | A | 152 | −7.707 | 33.487 | 17.435 | 1.00 | 23.89 |
| 1256 | CG2 | THR | A | 152 | −6.222 | 33.722 | 19.345 | 1.00 | 26.78 |
| 1257 | C | THR | A | 152 | −7.968 | 30.646 | 17.913 | 1.00 | 29.17 |
| 1258 | O | THR | A | 152 | −8.012 | 30.408 | 16.700 | 1.00 | 32.76 |
| 1259 | OXT | THR | A | 152 | −8.837 | 30.240 | 18.715 | 1.00 | 32.55 |
| 1261 | OH2 | WAT | W | 201 | −3.900 | 32.052 | 1.360 | 1.00 | 12.31 |
| 1262 | OH2 | WAT | W | 202 | 0.952 | 27.488 | −1.480 | 1.00 | 11.96 |
| 1263 | OH2 | WAT | W | 203 | 4.419 | 27.262 | 8.986 | 1.00 | 12.37 |
| 1264 | OH2 | WAT | W | 204 | −8.954 | 33.221 | 3.352 | 1.00 | 14.41 |
| 1265 | OH2 | WAT | W | 205 | −2.685 | 33.405 | 18.409 | 1.00 | 15.70 |
| 1266 | OH2 | WAT | W | 206 | −3.050 | 37.896 | 13.444 | 1.00 | 15.52 |
| 1267 | OH2 | WAT | W | 207 | −6.432 | 19.889 | −5.125 | 1.00 | 16.05 |
| 1268 | OH2 | WAT | W | 208 | 6.860 | 31.415 | 14.235 | 1.00 | 17.33 |
| 1269 | OH2 | WAT | W | 209 | −9.099 | 41.023 | 8.402 | 1.00 | 17.16 |
| 1270 | OH2 | WAT | W | 210 | −3.026 | 19.195 | 6.916 | 1.00 | 17.06 |
| 1271 | OH2 | WAT | W | 211 | −10.571 | 41.368 | −3.019 | 1.00 | 19.89 |
| 1272 | OH2 | WAT | W | 212 | 8.803 | 36.869 | 0.960 | 1.00 | 19.27 |
| 1273 | OH2 | WAT | W | 213 | 6.589 | 45.066 | 17.945 | 1.00 | 20.95 |
| 1274 | OH2 | WAT | W | 214 | 4.237 | 29.236 | 17.984 | 1.00 | 20.13 |
| 1275 | OH2 | WAT | W | 215 | 3.776 | 39.679 | 21.941 | 1.00 | 20.24 |
| 1276 | OH2 | WAT | W | 216 | 1.779 | 45.769 | 8.893 | 1.00 | 21.79 |
| 1277 | OH2 | WAT | W | 217 | 11.361 | 21.895 | 5.097 | 1.00 | 22.74 |
| 1278 | OH2 | WAT | W | 218 | −8.886 | 22.794 | 6.297 | 1.00 | 22.52 |
| 1279 | OH2 | WAT | W | 219 | −4.294 | 29.092 | −7.341 | 1.00 | 21.52 |
| 1280 | OH2 | WAT | W | 220 | −8.112 | 16.957 | 2.679 | 1.00 | 22.94 |
| 1281 | OH2 | WAT | W | 221 | 4.278 | 18.802 | 7.412 | 1.00 | 23.11 |
| 1282 | OH2 | WAT | W | 222 | 7.997 | 43.792 | 13.015 | 1.00 | 23.72 |
| 1283 | OH2 | WAT | W | 223 | −11.530 | 27.956 | 9.056 | 1.00 | 22.04 |
| 1284 | OH2 | WAT | W | 224 | −8.653 | 23.818 | 8.764 | 1.00 | 21.43 |
| 1285 | OH2 | WAT | W | 225 | 2.146 | 20.373 | 0.340 | 1.00 | 23.56 |
| 1286 | OH2 | WAT | W | 226 | 8.334 | 38.402 | 3.039 | 1.00 | 22.17 |
| 1287 | OH2 | WAT | W | 227 | 1.941 | 42.076 | 0.257 | 1.00 | 22.36 |
| 1288 | OH2 | WAT | W | 228 | −10.479 | 17.579 | 1.652 | 1.00 | 22.96 |
| 1289 | OH2 | WAT | W | 229 | −7.988 | 27.707 | −10.764 | 1.00 | 24.24 |
| 1290 | OH2 | WAT | W | 230 | −6.714 | 30.341 | −7.632 | 1.00 | 23.93 |
| 1291 | OH2 | WAT | W | 231 | −14.300 | 22.760 | −6.864 | 1.00 | 22.46 |
| 1292 | OH2 | WAT | W | 232 | −16.801 | 24.026 | −2.339 | 1.00 | 24.15 |
| 1293 | OH2 | WAT | W | 233 | 9.661 | 34.190 | 1.228 | 1.00 | 23.84 |
| 1294 | OH2 | WAT | W | 234 | −12.267 | 19.457 | −4.246 | 1.00 | 24.11 |
| 1295 | OH2 | WAT | W | 235 | 11.147 | 29.355 | 0.425 | 1.00 | 23.09 |
| 1296 | OH2 | WAT | W | 236 | 2.851 | 31.305 | 19.366 | 1.00 | 23.61 |
| 1297 | OH2 | WAT | W | 237 | −14.428 | 20.889 | −4.675 | 1.00 | 24.21 |
| 1298 | OH2 | WAT | W | 238 | −8.739 | 29.567 | −9.042 | 1.00 | 23.83 |
| 1299 | OH2 | WAT | W | 239 | −9.534 | 43.654 | 16.866 | 1.00 | 26.13 |
| 1300 | OH2 | WAT | W | 240 | 15.821 | 27.151 | 5.814 | 1.00 | 23.94 |
| 1301 | OH2 | WAT | W | 241 | −15.060 | 29.317 | −9.580 | 1.00 | 26.94 |
| 1302 | OH2 | WAT | W | 242 | −10.219 | 47.657 | 14.755 | 1.00 | 24.37 |
| 1303 | OH2 | WAT | W | 243 | 5.801 | 21.033 | −4.404 | 1.00 | 26.11 |
| 1304 | OH2 | WAT | W | 244 | 14.946 | 31.902 | 5.121 | 1.00 | 25.96 |
| 1305 | OH2 | WAT | W | 245 | −17.189 | 29.249 | 0.593 | 1.00 | 27.88 |
| 1306 | OH2 | WAT | W | 246 | −4.185 | 44.762 | 4.335 | 1.00 | 25.86 |
| 1307 | OH2 | WAT | W | 247 | −7.887 | 27.244 | 11.439 | 1.00 | 24.83 |
| 1308 | OH2 | WAT | W | 248 | −9.807 | 26.190 | 9.790 | 1.00 | 24.32 |
| 1309 | OH2 | WAT | W | 249 | 6.211 | 44.171 | 5.246 | 1.00 | 28.83 |
| 1310 | OH2 | WAT | W | 250 | 6.220 | 17.732 | 3.474 | 1.00 | 26.28 |
| 1311 | OH2 | WAT | W | 251 | −10.816 | 35.197 | −6.225 | 1.00 | 27.93 |
| 1312 | OH2 | WAT | W | 252 | −4.857 | 47.448 | 6.218 | 1.00 | 27.36 |
| 1313 | OH2 | WAT | W | 253 | 14.206 | 27.972 | 2.351 | 1.00 | 26.84 |
| 1314 | OH2 | WAT | W | 254 | −13.160 | 31.030 | −8.452 | 1.00 | 26.95 |
| 1315 | OH2 | WAT | W | 255 | 3.076 | 16.943 | 3.868 | 1.00 | 28.38 |
| 1316 | OH2 | WAT | W | 256 | −6.468 | 46.023 | 22.407 | 1.00 | 26.15 |
| 1317 | OH2 | WAT | W | 257 | −7.406 | 14.793 | −7.421 | 1.00 | 26.97 |
| 1318 | OH2 | WAT | W | 258 | −10.375 | 31.766 | −9.434 | 1.00 | 26.85 |
| 1319 | OH2 | WAT | W | 259 | −12.198 | 22.930 | −8.047 | 1.00 | 27.42 |
| 1320 | OH2 | WAT | W | 260 | −11.329 | 38.500 | −6.075 | 1.00 | 27.37 |
| 1321 | OH2 | WAT | W | 261 | −6.736 | 19.384 | −9.944 | 1.00 | 28.38 |
| 1322 | OH2 | WAT | W | 262 | −5.492 | 44.706 | 6.464 | 1.00 | 29.70 |
| 1323 | OH2 | WAT | W | 263 | −8.997 | 29.569 | 12.161 | 1.00 | 27.88 |
| 1324 | OH2 | WAT | W | 264 | −13.481 | 41.577 | −0.850 | 1.00 | 27.89 |
| 1325 | OH2 | WAT | W | 265 | −19.259 | 26.016 | 2.977 | 1.00 | 28.97 |
| 1326 | OH2 | WAT | W | 266 | −6.571 | 33.032 | −8.254 | 1.00 | 28.20 |
| 1327 | OH2 | WAT | W | 267 | −6.775 | 48.685 | 23.870 | 1.00 | 27.71 |
| 1328 | OH2 | WAT | W | 268 | −14.337 | 32.281 | 13.760 | 1.00 | 28.87 |
| 1329 | OH2 | WAT | W | 269 | 3.746 | 36.474 | −3.957 | 1.00 | 28.98 |
| 1330 | OH2 | WAT | W | 270 | −3.030 | 47.364 | 24.055 | 1.00 | 28.80 |
| 1331 | OH2 | WAT | W | 271 | −12.038 | 46.877 | −6.546 | 1.00 | 29.27 |
| 1332 | OH2 | WAT | W | 272 | 16.897 | 23.821 | 4.967 | 1.00 | 27.76 |
| 1333 | OH2 | WAT | W | 273 | −6.102 | 11.280 | 3.008 | 1.00 | 29.68 |
| 1334 | OH2 | WAT | W | 274 | −3.325 | 26.939 | −8.814 | 1.00 | 26.87 |
| 1335 | OH2 | WAT | W | 275 | −18.075 | 31.088 | 5.751 | 1.00 | 30.31 |
| 1336 | OH2 | WAT | W | 276 | 16.410 | 21.627 | 3.627 | 1.00 | 32.17 |
| 1337 | OH2 | WAT | W | 277 | −14.397 | 33.979 | −3.997 | 1.00 | 30.43 |
| 1338 | OH2 | WAT | W | 278 | 13.763 | 20.490 | 4.110 | 1.00 | 30.05 |
| 1339 | OH2 | WAT | W | 279 | 14.009 | 27.171 | 16.485 | 1.00 | 30.86 |
| 1340 | OH2 | WAT | W | 280 | −10.386 | 33.699 | 17.348 | 1.00 | 29.78 |
| 1341 | OH2 | WAT | W | 281 | 12.225 | 20.181 | 1.984 | 1.00 | 29.82 |
| 1342 | OH2 | WAT | W | 282 | 1.311 | 43.820 | 29.305 | 1.00 | 29.14 |
| 1343 | OH2 | WAT | W | 283 | 14.625 | 35.318 | 12.464 | 1.00 | 32.75 |
| 1344 | OH2 | WAT | W | 284 | 9.241 | 22.510 | 12.673 | 1.00 | 27.48 |
| 1345 | OH2 | WAT | W | 285 | −2.507 | 41.407 | 21.101 | 1.00 | 29.36 |
| 1346 | OH2 | WAT | W | 286 | −11.878 | 44.585 | 17.206 | 1.00 | 33.36 |
| 1347 | OH2 | WAT | W | 287 | 10.382 | 37.173 | 17.161 | 1.00 | 30.66 |
| 1348 | OH2 | WAT | W | 288 | 4.222 | 41.816 | 24.115 | 1.00 | 28.13 |
| 1349 | OH2 | WAT | W | 289 | 11.224 | 21.434 | 21.871 | 1.00 | 30.72 |
| 1350 | OH2 | WAT | W | 290 | −9.949 | 38.432 | 16.612 | 1.00 | 31.21 |
| 1351 | OH2 | WAT | W | 291 | 17.088 | 21.905 | 0.723 | 1.00 | 31.76 |
| 1352 | OH2 | WAT | W | 292 | −10.054 | 42.132 | 19.115 | 1.00 | 32.79 |
| 1353 | OH2 | WAT | W | 293 | 5.848 | 28.024 | −9.058 | 1.00 | 32.96 |
| 1354 | OH2 | WAT | W | 294 | 7.449 | 46.288 | 11.558 | 1.00 | 32.64 |
| 1355 | OH2 | WAT | W | 295 | −3.819 | 45.211 | 22.206 | 1.00 | 31.93 |
| 1356 | OH2 | WAT | W | 296 | −2.550 | 31.348 | −8.532 | 1.00 | 29.42 |
| 1357 | OH2 | WAT | W | 297 | 8.083 | 46.403 | 8.637 | 1.00 | 31.26 |
| 1358 | OH2 | WAT | W | 298 | −6.277 | 23.034 | 12.180 | 1.00 | 31.59 |
| 1359 | OH2 | WAT | W | 299 | −9.865 | 34.305 | −8.499 | 1.00 | 33.74 |
| 1360 | OH2 | WAT | W | 300 | −11.753 | 15.406 | −1.471 | 1.00 | 31.99 |
| 1361 | OH2 | WAT | W | 301 | 1.231 | 50.941 | 7.449 | 1.00 | 33.26 |
| 1362 | OH2 | WAT | W | 302 | 4.884 | 40.349 | 1.873 | 1.00 | 30.36 |
| 1363 | OH2 | WAT | W | 303 | −0.290 | 17.509 | 3.266 | 1.00 | 33.60 |
| 1364 | OH2 | WAT | W | 304 | −8.306 | 49.520 | 8.996 | 1.00 | 32.12 |
| 1365 | OH2 | WAT | W | 305 | −16.179 | 21.404 | −2.841 | 1.00 | 34.47 |
| 1366 | OH2 | WAT | W | 306 | −9.767 | 22.287 | 10.819 | 1.00 | 33.82 |
| 1367 | OH2 | WAT | W | 307 | 2.605 | 25.493 | 23.695 | 1.00 | 32.70 |
| 1368 | OH2 | WAT | W | 308 | −1.233 | 29.153 | 22.803 | 1.00 | 31.89 |
| 1369 | OH2 | WAT | W | 309 | 3.294 | 43.277 | 31.535 | 1.00 | 30.01 |
| 1370 | OH2 | WAT | W | 310 | −18.145 | 32.995 | 3.738 | 1.00 | 31.44 |
| 1371 | OH2 | WAT | W | 311 | 3.862 | 47.222 | 7.780 | 1.00 | 34.38 |
| 1372 | OH2 | WAT | W | 312 | 1.401 | 19.720 | 3.009 | 1.00 | 32.88 |
| 1373 | OH2 | WAT | W | 313 | 2.362 | 48.671 | 6.269 | 1.00 | 29.57 |
| 1374 | OH2 | WAT | W | 314 | −8.726 | 15.367 | 4.938 | 1.00 | 31.71 |
| 1375 | OH2 | WAT | W | 315 | −17.604 | 30.244 | −9.311 | 1.00 | 31.67 |
| 1376 | OH2 | WAT | W | 316 | 6.049 | 23.620 | −7.356 | 1.00 | 30.98 |
| 1377 | OH2 | WAT | W | 317 | −17.887 | 17.323 | −5.165 | 1.00 | 29.81 |
| 1378 | OH2 | WAT | W | 318 | 0.756 | 45.919 | 28.072 | 1.00 | 34.09 |
| 1379 | OH2 | WAT | W | 319 | 4.224 | 50.319 | 18.097 | 1.00 | 36.06 |
| 1380 | OH2 | WAT | W | 320 | −1.807 | 34.389 | −7.302 | 1.00 | 32.63 |
| 1381 | OH2 | WAT | W | 321 | −5.641 | 26.448 | 16.631 | 1.00 | 34.55 |
| 1382 | OH2 | WAT | W | 322 | −8.023 | 11.775 | −5.519 | 1.00 | 37.17 |
| 1383 | OH2 | WAT | W | 323 | 0.906 | 27.929 | 21.790 | 1.00 | 31.90 |
| 1384 | OH2 | WAT | W | 324 | 16.865 | 27.638 | 12.993 | 1.00 | 39.01 |
| 1385 | OH2 | WAT | W | 325 | 13.149 | 32.235 | 10.074 | 1.00 | 30.65 |
| 1386 | OH2 | WAT | W | 326 | 0.881 | 52.908 | 5.493 | 1.00 | 36.21 |
| 1387 | OH2 | WAT | W | 327 | −11.094 | 35.858 | 15.788 | 1.00 | 35.61 |
| 1388 | OH2 | WAT | W | 328 | 9.464 | 44.941 | 14.828 | 1.00 | 35.35 |
| 1389 | OH2 | WAT | W | 329 | 11.956 | 33.216 | −1.753 | 1.00 | 35.31 |
| 1390 | OH2 | WAT | W | 330 | −15.633 | 35.904 | −1.678 | 1.00 | 34.57 |
| 1391 | OH2 | WAT | W | 331 | 0.433 | 18.388 | −5.721 | 1.00 | 33.25 |
| 1392 | OH2 | WAT | W | 332 | −3.090 | 22.685 | 15.873 | 1.00 | 36.98 |
| 1393 | OH2 | WAT | W | 333 | −17.189 | 23.616 | 6.052 | 1.00 | 32.38 |
| 1394 | OH2 | WAT | W | 334 | 17.579 | 18.631 | 10.887 | 1.00 | 35.84 |
| 1395 | OH2 | WAT | W | 335 | −0.520 | 28.452 | −8.229 | 1.00 | 35.62 |
| 1396 | OH2 | WAT | W | 336 | 5.589 | 22.680 | 18.766 | 1.00 | 35.02 |
| 1397 | OH2 | WAT | W | 337 | −5.195 | 11.581 | −7.734 | 1.00 | 38.68 |
| 1398 | OH2 | WAT | W | 338 | 14.660 | 30.377 | 1.229 | 1.00 | 35.10 |
| 1399 | OH2 | WAT | W | 339 | −17.723 | 20.205 | −0.873 | 1.00 | 34.14 |
| 1400 | OH2 | WAT | W | 340 | 12.282 | 40.815 | 19.756 | 1.00 | 39.51 |
| 1401 | OH2 | WAT | W | 341 | −16.095 | 49.517 | 2.071 | 1.00 | 37.48 |
| 1402 | OH2 | WAT | W | 342 | 16.962 | 24.111 | −1.316 | 1.00 | 37.77 |
| 1403 | OH2 | WAT | W | 343 | 8.916 | 39.469 | 21.199 | 1.00 | 34.40 |
| 1404 | OH2 | WAT | W | 344 | −20.253 | 29.440 | 4.304 | 1.00 | 37.94 |
| 1405 | OH2 | WAT | W | 345 | −3.441 | 21.151 | 18.584 | 1.00 | 33.63 |
| 1406 | OH2 | WAT | W | 346 | 11.274 | 39.429 | 3.812 | 1.00 | 39.15 |
| 1407 | OH2 | WAT | W | 347 | 5.930 | 40.904 | −1.475 | 1.00 | 35.58 |
| 1408 | OH2 | WAT | W | 348 | −15.467 | 35.778 | 5.107 | 1.00 | 36.50 |
| 1409 | OH2 | WAT | W | 349 | 1.737 | 30.364 | −8.724 | 1.00 | 37.24 |
| 1410 | OH2 | WAT | W | 350 | −4.187 | 22.695 | 21.115 | 1.00 | 35.40 |
| 1411 | OH2 | WAT | W | 351 | −11.792 | 25.223 | 11.653 | 1.00 | 34.67 |
| 1412 | OH2 | WAT | W | 352 | 13.323 | 31.807 | 2.854 | 1.00 | 34.62 |
| 1413 | OH2 | WAT | W | 353 | 1.228 | 19.080 | −2.076 | 1.00 | 36.49 |
| 1414 | OH2 | WAT | W | 354 | −18.253 | 20.118 | −5.099 | 1.00 | 39.85 |
| 1415 | OH2 | WAT | W | 355 | −15.224 | 37.809 | 6.369 | 1.00 | 38.11 |
| 1416 | OH2 | WAT | W | 356 | −15.750 | 43.843 | 6.998 | 1.00 | 38.78 |
| 1417 | OH2 | WAT | W | 357 | 8.688 | 23.641 | −7.129 | 1.00 | 40.34 |
| 1418 | OH2 | WAT | W | 358 | 6.766 | 40.676 | 3.480 | 1.00 | 37.27 |
| 1419 | OH2 | WAT | W | 359 | 10.089 | 26.035 | −7.559 | 1.00 | 37.40 |
| 1420 | OH2 | WAT | W | 360 | −1.653 | 34.642 | −10.325 | 1.00 | 41.05 |
| 1421 | OH2 | WAT | W | 361 | 11.902 | 35.717 | 14.116 | 1.00 | 38.61 |
| 1422 | OH2 | WAT | W | 362 | 5.278 | 13.387 | 6.098 | 1.00 | 38.64 |
| 1423 | OH2 | WAT | W | 363 | 1.897 | 18.206 | −8.382 | 1.00 | 39.07 |
| 1424 | OH2 | WAT | W | 364 | −4.846 | 26.666 | −10.725 | 1.00 | 38.17 |
| 1425 | OH2 | WAT | W | 365 | 9.544 | 22.055 | −3.166 | 1.00 | 37.03 |
| 1426 | OH2 | WAT | W | 366 | −13.428 | 31.452 | 19.752 | 1.00 | 40.02 |
| 1427 | OH2 | WAT | W | 367 | −3.675 | 19.985 | −11.673 | 1.00 | 39.56 |
| 1428 | OH2 | WAT | W | 368 | 5.940 | 48.016 | 13.223 | 1.00 | 36.94 |
| 1429 | OH2 | WAT | W | 369 | −15.068 | 43.053 | −2.748 | 1.00 | 38.98 |
| 1430 | OH2 | WAT | W | 370 | 17.423 | 44.152 | 4.412 | 1.00 | 41.67 |
| 1431 | OH2 | WAT | W | 371 | −9.559 | 20.801 | 4.372 | 1.00 | 38.63 |
| 1432 | OH2 | WAT | W | 372 | −17.485 | 40.033 | −1.895 | 1.00 | 42.67 |
| 1433 | OH2 | WAT | W | 373 | 10.911 | 38.587 | 20.026 | 1.00 | 41.59 |
| 1434 | OH2 | WAT | W | 374 | 5.617 | 16.185 | 5.471 | 1.00 | 38.51 |
| 1435 | OH2 | WAT | W | 375 | 5.058 | 38.594 | −5.007 | 1.00 | 37.41 |
| 1436 | OH2 | WAT | W | 376 | 7.267 | 44.085 | 25.369 | 1.00 | 41.82 |
| 1437 | OH2 | WAT | W | 377 | 17.288 | 38.270 | 7.853 | 1.00 | 37.77 |
| 1438 | OH2 | WAT | W | 378 | 10.919 | 19.053 | 11.527 | 1.00 | 39.48 |
| 1439 | OH2 | WAT | W | 379 | 18.280 | 39.031 | 10.623 | 1.00 | 37.56 |
| 1440 | OH2 | WAT | W | 380 | 8.958 | 43.761 | 17.287 | 1.00 | 39.15 |
| 1441 | OH2 | WAT | W | 381 | −11.761 | 40.530 | 9.983 | 1.00 | 40.41 |
| 1442 | OH2 | WAT | W | 382 | 8.645 | 25.729 | 21.957 | 1.00 | 41.15 |
| 1443 | OH2 | WAT | W | 383 | 12.776 | 23.515 | −3.174 | 1.00 | 39.26 |
| 1444 | OH2 | WAT | W | 384 | 1.500 | 48.497 | 0.811 | 1.00 | 42.98 |
| 1445 | OH2 | WAT | W | 385 | 6.410 | 25.470 | −8.821 | 1.00 | 42.34 |
| 1446 | OH2 | WAT | W | 386 | 18.547 | 41.167 | 9.409 | 1.00 | 38.96 |
| 1447 | OH2 | WAT | W | 387 | −18.337 | 35.945 | 7.027 | 1.00 | 41.21 |
| 1448 | OH2 | WAT | W | 388 | −17.146 | 19.303 | 1.205 | 1.00 | 41.48 |
| 1449 | OH2 | WAT | W | 389 | −8.236 | 20.789 | 12.609 | 1.00 | 42.25 |
| 1450 | OH2 | WAT | W | 390 | −14.693 | 18.506 | 2.086 | 1.00 | 43.16 |
| 1451 | OH2 | WAT | W | 391 | 10.531 | 22.385 | 17.929 | 1.00 | 38.36 |
| 1452 | OH2 | WAT | W | 392 | 5.923 | 36.758 | −6.984 | 1.00 | 43.87 |
| 1453 | OH2 | WAT | W | 393 | −4.095 | 47.470 | −0.042 | 1.00 | 41.94 |
| 1454 | OH2 | WAT | W | 394 | 2.227 | 24.121 | −10.040 | 1.00 | 42.58 |
| 1455 | OH2 | WAT | W | 395 | 0.739 | 16.533 | −3.072 | 1.00 | 43.06 |
| 1456 | OH2 | WAT | W | 396 | 8.462 | 45.631 | 21.908 | 1.00 | 37.63 |
| 1457 | OH2 | WAT | W | 397 | 3.389 | 38.004 | −10.331 | 1.00 | 43.14 |
| 1458 | OH2 | WAT | W | 398 | −4.299 | 38.307 | −7.880 | 1.00 | 43.62 |
| 1459 | OH2 | WAT | W | 399 | −12.240 | 29.023 | 19.389 | 1.00 | 45.17 |
| 1460 | OH2 | WAT | W | 400 | 7.768 | 47.162 | 23.911 | 1.00 | 45.18 |
| 1461 | OH2 | WAT | W | 401 | −15.550 | 21.715 | 6.543 | 1.00 | 45.05 |
| 1462 | OH2 | WAT | W | 402 | −13.415 | 46.294 | 12.126 | 1.00 | 39.05 |
| 1463 | OH2 | WAT | W | 403 | −7.012 | 9.727 | 0.551 | 1.00 | 31.21 |
| 1464 | OH2 | WAT | W | 404 | −6.831 | 45.423 | −0.004 | 1.00 | 44.32 |
| 1465 | OH2 | WAT | W | 405 | 1.401 | 36.546 | −5.582 | 1.00 | 36.83 |
| 1466 | OH2 | WAT | W | 406 | 6.714 | 40.039 | 5.978 | 1.00 | 26.38 |
| 1467 | OH2 | WAT | W | 407 | 1.846 | 26.595 | −12.694 | 1.00 | 41.70 |
| 1468 | OH2 | WAT | W | 408 | 15.551 | 16.798 | 9.433 | 1.00 | 39.88 |
| 1469 | OH2 | WAT | W | 409 | −13.045 | 36.935 | 17.563 | 1.00 | 38.24 |
| 1470 | OH2 | WAT | W | 410 | −15.712 | 34.236 | 10.684 | 1.00 | 35.00 |
| 1471 | OH2 | WAT | W | 411 | 8.412 | 28.930 | −8.302 | 1.00 | 34.98 |
| 1472 | OH2 | WAT | W | 412 | −9.821 | 38.455 | −8.616 | 1.00 | 37.51 |
| 1473 | OH2 | WAT | W | 413 | 6.183 | 39.366 | 22.141 | 1.00 | 33.61 |
| 1474 | OH2 | WAT | W | 414 | −8.491 | 25.915 | 18.008 | 1.00 | 39.16 |
| 1475 | OH2 | WAT | W | 415 | 12.368 | 34.247 | 1.800 | 1.00 | 34.92 |
| 1476 | OH2 | WAT | W | 416 | −1.315 | 11.452 | −6.334 | 1.00 | 38.39 |
| 1477 | OH2 | WAT | W | 417 | 15.835 | 15.071 | 15.103 | 1.00 | 41.32 |
| 1478 | OH2 | WAT | W | 418 | −8.602 | 41.350 | 15.721 | 1.00 | 28.93 |
| 1479 | OH2 | WAT | W | 419 | −16.014 | 40.490 | 2.677 | 1.00 | 39.46 |
| 1480 | OH2 | WAT | W | 420 | −19.535 | 29.475 | −2.875 | 1.00 | 40.49 |
| 1481 | OH2 | WAT | W | 421 | 12.703 | 39.037 | 5.886 | 1.00 | 42.65 |
| 1482 | OH2 | WAT | W | 422 | 10.637 | 20.682 | 13.781 | 1.00 | 34.75 |
| 1483 | OH2 | WAT | W | 423 | 10.352 | 21.563 | −6.884 | 1.00 | 39.85 |
| 1484 | OH2 | WAT | W | 424 | −5.568 | 46.943 | 1.853 | 1.00 | 39.37 |
| 1485 | OH2 | WAT | W | 425 | 13.509 | 35.976 | 3.341 | 1.00 | 40.52 |
| 1486 | OH2 | WAT | W | 426 | 7.916 | 20.112 | −8.973 | 1.00 | 42.90 |
| 1487 | OH2 | WAT | W | 427 | 9.277 | 41.681 | 1.669 | 1.00 | 43.21 |
| 1488 | OH2 | WAT | W | 428 | 0.833 | 18.171 | 1.190 | 1.00 | 42.90 |
| 1489 | OH2 | WAT | W | 429 | 18.908 | 47.454 | 5.692 | 1.00 | 45.51 |
| 1490 | OH2 | WAT | W | 430 | −9.037 | 22.244 | 15.522 | 1.00 | 39.57 |

What is claimed is:

1. A composition comprising a murine IL-18 in crystalline form.

2. A crystal of murine IL-18, wherein said crystal effectively diffracts x-rays for the determination of the atomic coordinates of said murine IL-18 to a resolution of between about 1.3 Å and 5.0 Å.

3. The crystal as claimed in claim 2 that effectively diffracts x-rays for the determination of the atomic coordinates of said murine IL-18 to a resolution of between about 1.3 Å and 3.0 Å.

4. The crystal as claimed in claim 3 that effectively diffracts x-rays for the determination of the atomic coordinates of said murine IL-18 to a resolution of between about 1.3 Å and 2.5 Å.

5. The composition as claimed in claim 1, wherein said murine IL-18 is an essentially pure native form or a homolog thereof.

6. The composition as claimed in claim 1,
wherein said composition has one or more patches of its surface in contact with its receptor;
wherein said patches are lined by three groups of amino acid residues;
wherein said first group comprises Ile15-Gln18, Val22-Pro27, Asp31-Asp36, Glu128-Ala131, Lys145-Met148;
wherein said second group comprises Gly3-Thr8, Met 50-Gly58, Glu89-Asp92; and
wherein said third group comprises Lys103-Asn109.

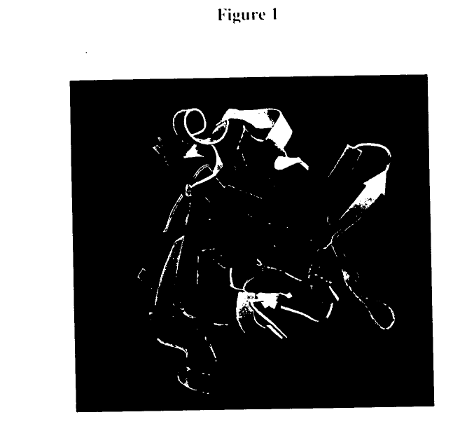

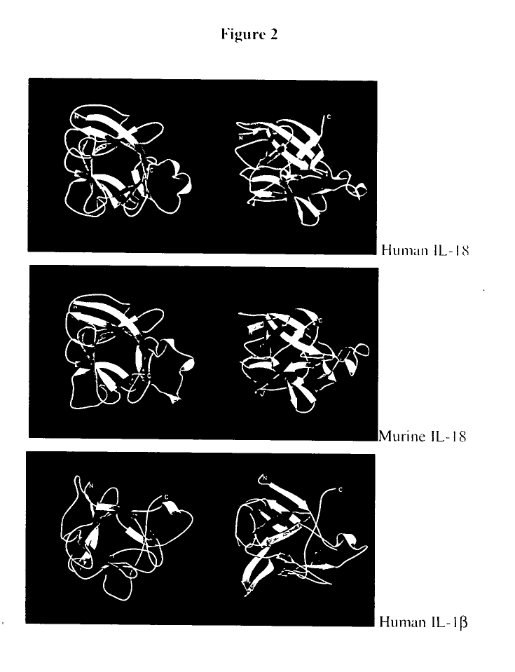

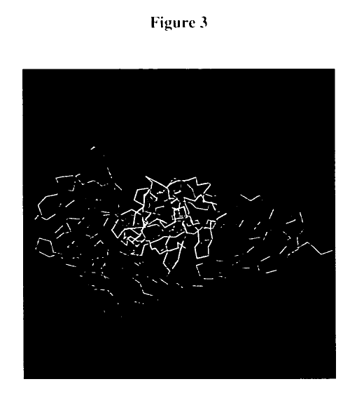

7. A heavy atom derivative of the composition in claim 1, wherein said derivative comprises a protein having the coordinates in Table I. and in FIGS. 1-3.

8. The composition as claimed in claim 2, wherein said murine IL-18 is characterized by a β-trefoil fold.

9. A process of identifying an agonist or an antagonist molecule of murine IL-18, comprising an entity selected from the group consisting of: a peptide, a non-peptide molecule and a chemical compound;
wherein said molecule is capable of enhancing, eliciting or blocking the biological activity resulting from interaction with the murine IL-18 and its receptor;
wherein said process comprises:
introducing into a suitable computer program parameters defining an interacting surface based on the conformation of murine IL-18 corresponding to the coordinates of FIGS. 1-3 and Table I.;
wherein said program displays the three-dimensional structure thereof;
creating a three-dimensional structure of a test compound in said computer program;
displaying an superimposing the model of said test compound on the model.
assessing whether said test compound model fits spatially into the binding site;
incorporating said test compound in a biological cytokine activity assay; and
determining whether said test compound inhibits or enhances the biological activity of murine IL-18.

10. A process of identifying an agonist or an antagonist capable of modifying the biological activity of the composition of claim 1, wherein said process comprises:
carrying out an in vitro assay by introducing said compound into a biological cytokine activity assay mixture; and
determining whether said test compound inhibits or enhances the biological activity of murine IL-18 receptor.

11. A molecule identified by the method of claim 9, wherein said molecule is selected from a group consisting of: a peptide, a peptidomimetic and a synthetic compound.

12. A molecule identified by the method of claim 10, wherein said molecule is selected from a group consisting of: a peptide, a peptidomimetic and a synthetic compound.

13. The molecule as claimed in claim 11, wherein said molecule is selected from a group consisting of: an antagonist and an agonist.

14. The molecule as claimed in claim 12, wherein said molecule is selected from a group consisting of: an antagonist and an agonist.

15. A method of determining a crystal structure, said method comprising the steps of: using the structural coordinates of a murine IL-18 crystal or portions thereof, and determining the structure coordinates of a mutant, homologue or co-complex of said murine IL-18 by molecular replacement.

16. A method of drug design comprising the use of the atomic coordinates of a murine IL-18 crystal to computationally evaluate a chemical entity for association with the receptor binding site of murine IL-18.

17. The method as claimed in claim 16, wherein said entity is an agonist or an antagonist of murine IL-18.

18. The method of drug design as claimed in claim 16, wherein said method comprises the step of: using the structure coordinates of murine IL-18 to identify an intermediate in a chemical reaction between murine IL-18 and a compound which is a ligand of said murine IL-18.

19. The method as claimed in claim 16, wherein said structure coordinates comprises the coordinates of FIGS. 1-3 and Table I.

20. The method as claimed in claim 17, wherein said structure coordinates comprises the coordinates of FIGS. 1-3 and Table I.

21. The method as claimed in claim 18, wherein said structure coordinates comprises the coordinates of FIGS. 1-3 and Table I.
